(19) United States
(12) Patent Application Publication
(10) Pub. No.: US 2025/0000091 A1
(43) Pub. Date: Jan. 2, 2025

(54) GRAFTABLE BIOCIDAL LINKERS AND POLYMERS AND USES THEREOF

(71) Applicant: DEBOGY MOLECULAR, INC., Battle Creek, MI (US)

(72) Inventor: Othman BOULOUSSA, Bordeaux (FR)

(73) Assignee: DEBOGY MOLECULAR, INC., Battle Creek, MI (US)

(21) Appl. No.: 18/000,777

(22) PCT Filed: Jun. 5, 2021
(86) PCT No.: PCT/US2021/036048

§ 371 Date: Dec. 5, 2022

Related U.S. Application Data
(60) Provisional application No. 63/166,118, filed on Mar. 25, 2021.
(60) Provisional application No. 63/166,113, filed on Mar. 25, 2021.
(60) Provisional application No. 63/035,635, filed on Jun. 5, 2020.
(60) Provisional application No. 63/035,628, filed on Jun. 5, 2020.

(51) Int. Cl. A01N 43/40; A01N 33/12; A01P 1/00; A01P 3/00; C08F 26/06; C08F 212/14

(52) U.S. Cl. CPC A01N 43/40; A01N 33/12; A01P 1/00; A01P 3/00; C08F 26/06; C08F 212/28

(57) ABSTRACT

Ready-to-graft polymers and compounds, surfaces grafted to same, and methods of making and using the same for controlling the growth of at least one bacteria, fungi, protozoa, or virus are disclosed.

### CROSS REFERENCE TO RELATED APPLICATIONS

The present application claims priority to U.S. Provisional Patent Application Nos. 63/035,635, filed Jun. 5, 2020, 63/035,628, filed Jun. 5, 2020, 63/166,113, filed Mar. 25, 2021, and 63/166,118, filed Mar. 25, 2021, the contents of all of which are incorporated by reference herein in their entireties.

### FIELD

The disclosure relates generally to novel graftable linkers, such as catechol and dipodal silane linkers, and polymers having both biocidal and biocompatibility properties, methods of preparation of same, and methods of grafting same on surfaces to prevent and reduce the colonization and proliferation of germs (e.g. bacteria, viruses, and fungi) on surfaces and surfaces.

### BACKGROUND

Biocidal polymers are becoming increasingly important in order to contain and control the spread of infectious pathogens in a variety of health and industrial applications. To this end, biocidal polymers have been developed for use in solution form as well as to incorporate biocidal activity onto materials via coatings.

It would be highly desirable to have a solution of a biocidal polymer having both biocidal and biocompatibility properties for a prolonged storage period. It is, therefore, advisable to have a ready-to-use biocidal product which prevents fast reticulation in volume and thereby prolonging storage period.

### SUMMARY

In one aspect, the disclosure provides a polymer comprising at least one moiety of formula (XVIIa):

In some embodiments, the polymer includes at least one moiety of formula (XVIIa):

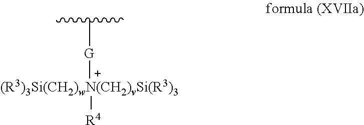

wherein in formula (XVIIa):
G is a single bond or a linking group;
each R³ is independently optionally substituted alkoxy;
R⁴ is independently optionally substituted alkyl;
v is an integer from 3 to 10; and
w is an integer from 3 to 10.

In some embodiments, the polymer includes at least one moiety of formula (XVIIb) or formula (XVIIe):

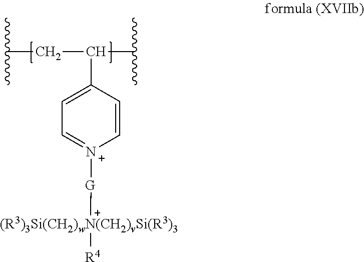

wherein in formula (XVIIb):
G is a single bond or a linking group;
each R³ is independently optionally substituted alkoxy;
R⁴ is independently optionally substituted alkyl;
v is an integer from 3 to 10; and
w is an integer from 3 to 10;

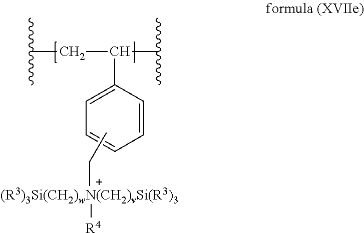

wherein in formula (XVIIe):
each R³ is independently optionally substituted alkoxy;
R⁴ is independently optionally substituted alkyl;
v is an integer from 3 to 10; and
w is an integer from 3 to 10.

In some embodiments, the polymer further includes at least one moiety of formula (III):

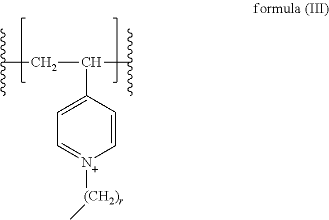

wherein in formula (III):
r is an integer from 3 to 20.

In some embodiments, the polymer includes at least one moiety of formula (XVIIc):

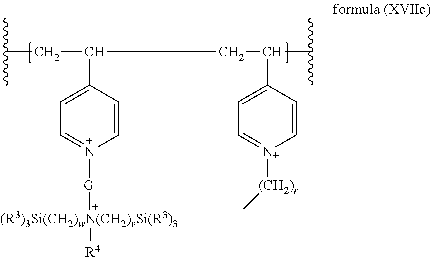

wherein in formula (XVIIc):
G is a single bond or a linking group;
each R³ is independently optionally substituted alkoxy;
R⁴ is independently optionally substituted alkyl;
r is an integer from 3 to 11;
v is an integer from 3 to 10; and
w is an integer from 3 to 10.

In some embodiments, the polymer further includes at least one moiety of formula (VII):

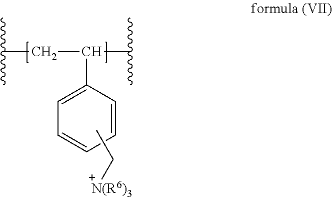

wherein in formula (VII):
each R⁶ is independently optionally substituted alkyl.

In some embodiments, the polymer comprises at least one moiety of formula (XVIIg):

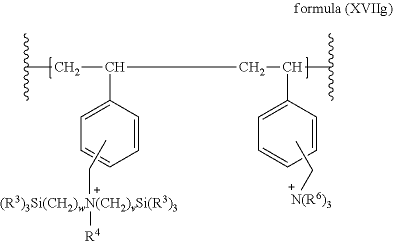

wherein in formula (XVIIg):
each R³ is independently optionally substituted alkoxy;
R⁴ is independently optionally substituted alkyl;
v is an integer from 3 to 10; and
w is an integer from 3 to 10.

In some embodiments, the polymer further includes at least one moiety of formula (XVIIh):

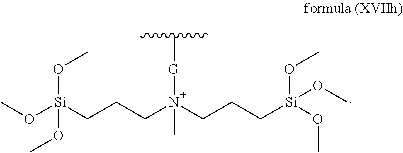

In some embodiments, the polymer includes at least one moiety of formula (XVIIf):

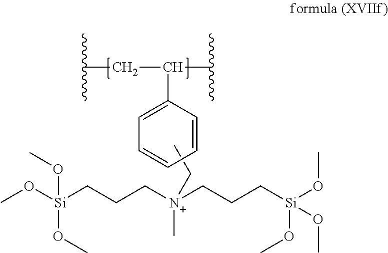

wherein in formula (XVIIf):
each R² is independently optionally substituted alkyl. In some embodiments, each R² is independently a C₁-C₄ alkyl.

In some embodiments, the polymer includes least one moiety of formula (XVIId):

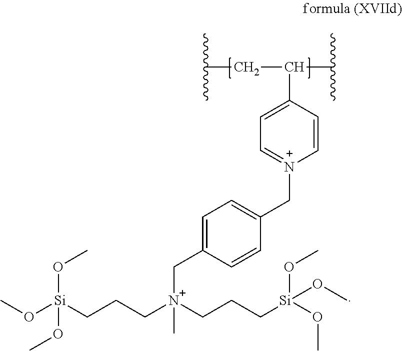

In one aspect, the disclosure includes at least one moiety of formula (XXI):

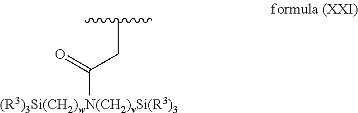

wherein in formula (XXI):
each R³ is independently optionally substituted alkoxy;
v is an integer from 3 to 10; and
w is an integer from 3 to 10.

In some embodiments, the polymer includes at least one moiety of formula (XXIa) or formula (XXII):

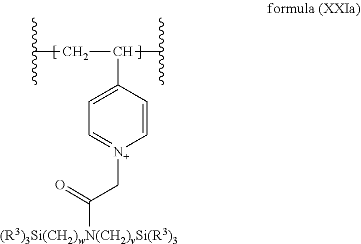

wherein in formula (XXIa):
each R³ is independently optionally substituted alkoxy;
v is an integer from 3 to 10; and
w is an integer from 3 to 10;

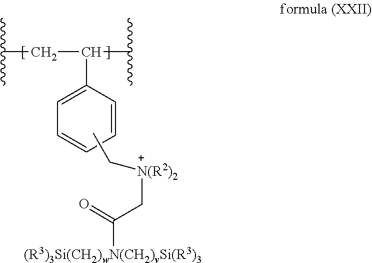

wherein in formula (XXII):
each R² is independently optionally substituted alkyl;
each R³ is independently optionally substituted alkoxy;
v is an integer from 3 to 10; and
w is an integer from 3 to 10.

In some embodiments, the polymer includes at least one moiety of formula (XXIb):

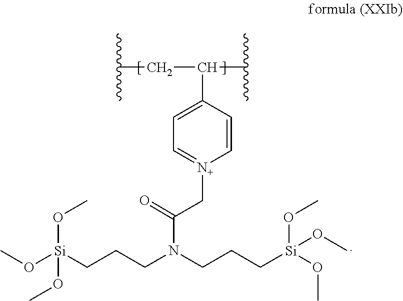

In some embodiments, the polymer includes at least one moiety of formula (III):

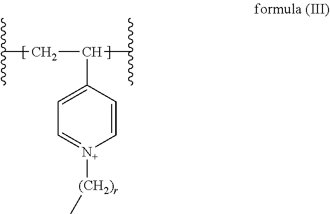

wherein r is an integer from 3 to 20.

In some embodiments, the polymer includes at least one moiety of formula (XXId):

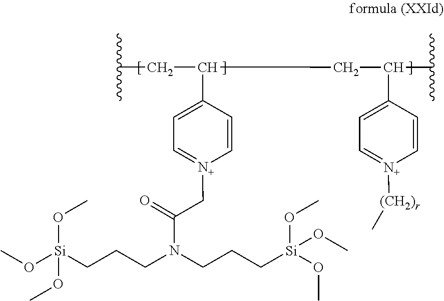

wherein in formula (XXId):
r is an integer from 3 to 11

In some embodiments, the polymer includes at least one moiety of formula (XXIIa):

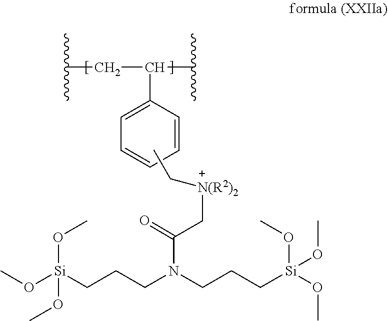

wherein in formula (XXII):
each R² is independently optionally substituted alkyl.

In some embodiments, the polymer includes further at least one moiety of formula (VII):

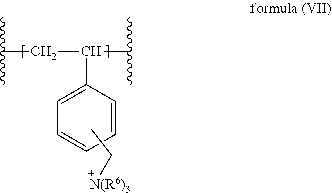

wherein in formula (VII):
each R⁶ is independently optionally substituted alkyl.

In some embodiments, the polymer includes at least one moiety of formula (XXIIc):

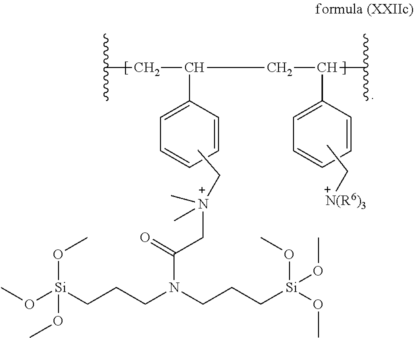

In one aspect, the invention includes a polymer comprising at least one moiety of formula (II):

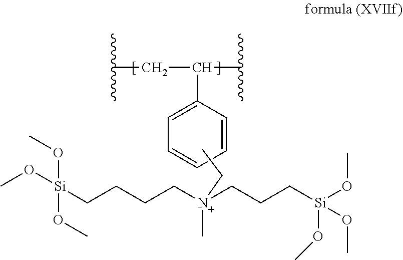

wherein in formula (XVIIf):
each R² is independently optionally substituted alkyl. In some embodiments, each R² is independently a C₁-C₄ alkyl.

In one aspect, the disclosure includes at least one moiety of formula (XVIIf):

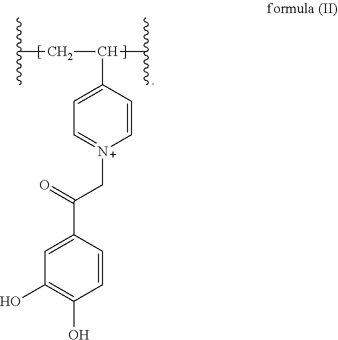 [SMILES: *CC(*)c1cc[n+](CC(=O)c2ccc(O)c(O)c2)cc1]

In some embodiments, the polymer includes at least one moiety of formula (III):

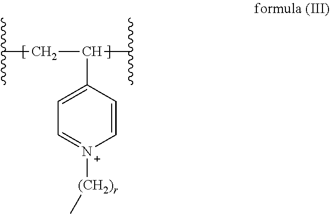

wherein r is an integer from 3 to 20.

In some embodiments, the polymer includes at least one moiety of formula (IV):

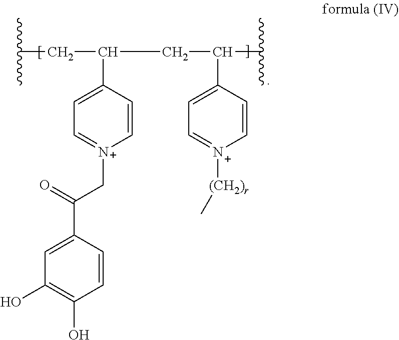

wherein in formula (IV):
r is an integer from 3 to 11.

In one aspect, the disclosure provides a polymer comprising at least one moiety of formula (V):

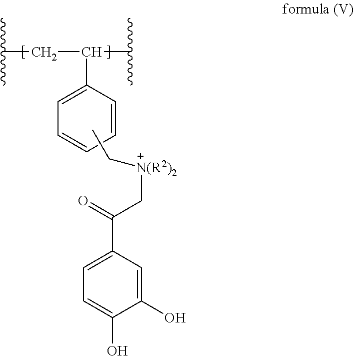

wherein in formula (V):
each R² is independently optionally substituted alkyl.

In some embodiments, the polymer comprises at least one moiety of formula (VI):

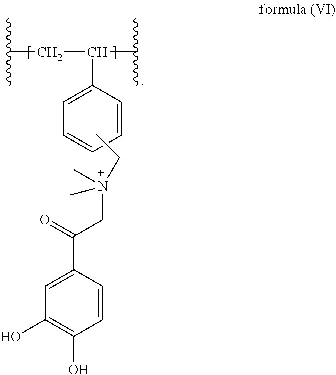

In some embodiments, the polymer further comprises a moiety of formula (VII):

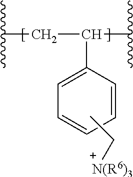

wherein in formula (VII):
each R⁶ is independently optionally substituted alkyl.

In some embodiments, the polymer further includes at least one moiety of formula (VIII):

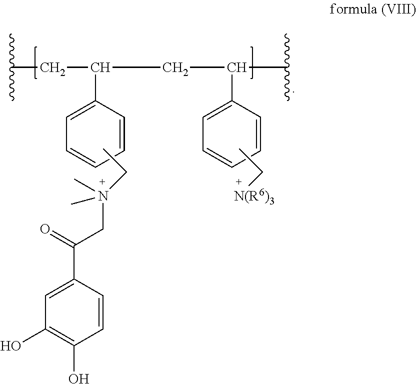

wherein in formula (VIII):
each R⁶ is independently optionally substituted alkyl.

In some embodiments, the polymer includes polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide), poly(diallyldimethylammonium), or a C₃-C₂₂ alkyne.

In one aspect, the disclosure provides a polyethylenimine (PEI) polymer comprising at least one moiety of formula (IXa), formula (IXb), or formula (IXh):

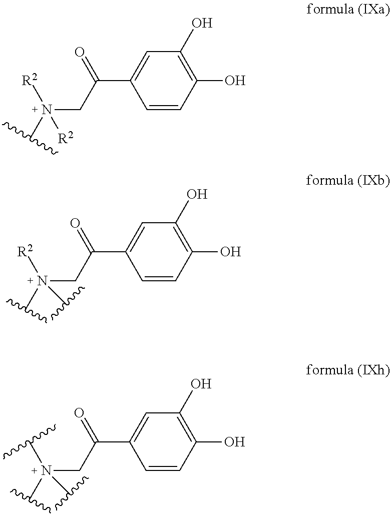

wherein in formula (IXa), formula (IXb), and formula (IXh):
each R² is independently optionally substituted alkyl.

In some embodiments, the PEI polymer includes at least one moiety of formula (IXc) or (IXd):

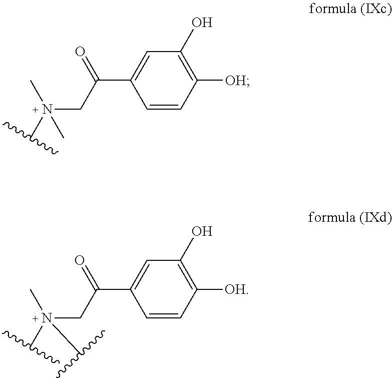

In some embodiments, the PEI polymer includes at least one moiety of formula (IXe), or any substructure thereof:

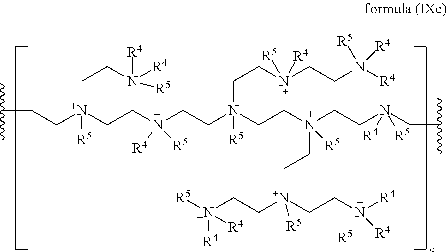

wherein in formula (IXe):
each R⁴ is independently optionally substituted alkyl; and
each R⁵ is independently optionally substituted alkyl or a moiety of formula (Ia):

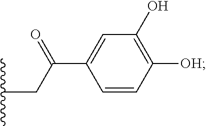 [SMILES: *CC(=O)c1ccc(O)c(O)c1]

with the proviso that at least one R⁵ is a moiety of formula (Ia):

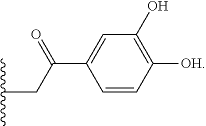 [SMILES: *CC(=O)c1ccc(O)c(O)c1]

In one aspect, the disclosure includes a polyethylenimine (PEI) polymer comprising at least one moiety of formula (IXf), or any substructure thereof:

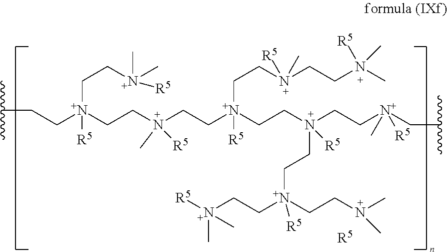

wherein in formula (IXf):
each R⁵ is independently C₁₀ alkyl or

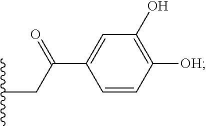 [SMILES: *CC(=O)c1ccc(O)c(O)c1]

with the proviso that at least one R⁵ is a moiety of formula (Ia):

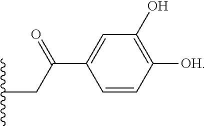 [SMILES: *CC(=O)c1ccc(O)c(O)c1]

In one aspect, the disclosure provides a polyethylenimine (PEI) polymer comprising at least one moiety of formula (IXg), or any substructure thereof:

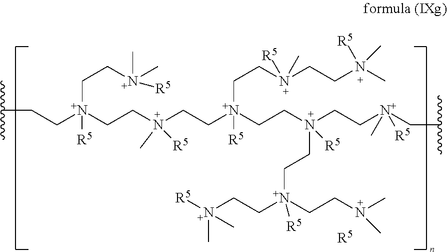

wherein in formula (IXg):

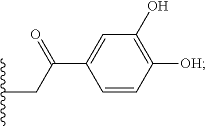 [SMILES: *CC(=O)c1ccc(O)c(O)c1]

each R⁵ is independently C₆ alkyl or

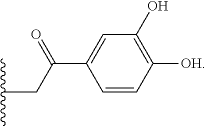 [SMILES: *CC(=O)c1ccc(O)c(O)c1]

with the proviso that at least one R⁵ is a moiety of formula (Ia):

In some embodiments, the PEI polymer comprises at least one moiety of formula (XIb), or any substructure thereof:

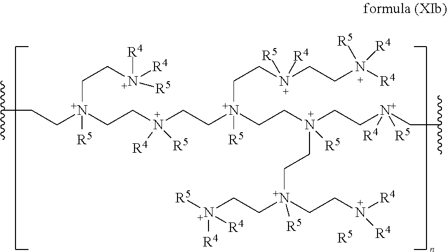

wherein in formula (XIb):
each R⁴ is independently optionally substituted alkyl; and
each R⁵ is independently optionally substituted alkyl or a moiety of formula (XIa):

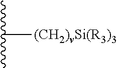

each R³ is independently optionally substituted alkoxy; and
v is an integer from 3 to 10,
   with the proviso that at least one R⁵ is a moiety of formula (XIa):

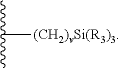

In one aspect, the disclosure provides a polyethylenimine (PEI) polymer comprising at least one moiety of formula (XIc), or any substructure thereof:

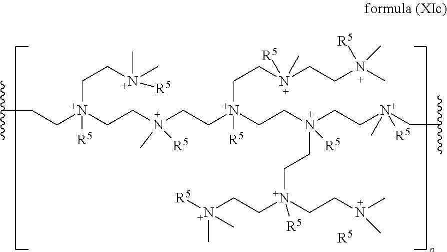

wherein in formula (XIc):
each R⁵ is independently C₆ alkyl or

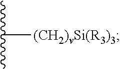

each R³ is independently optionally substituted alkoxy; and
v is an integer from 3 to 10,
   with the proviso that at least one R⁵ is

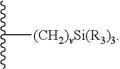

In some embodiments, each moiety of formula (XIa)

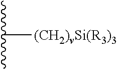

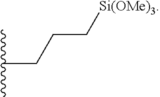

In one aspect, the disclosure provides a compound of formula (XL):

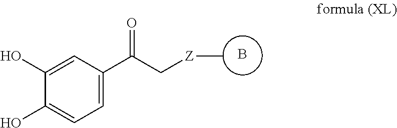

wherein in formula (XL):
Z is a single bond or a linking group; and
B is a biomolecule

In some embodiments, the compound of formula (XL) is a compound of formula (XLa):

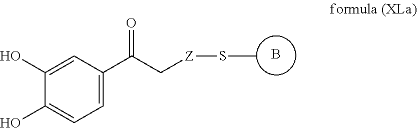

wherein in formula (XLa):
Z is a single bond or a linking group; and
B is a biomolecule

In some embodiments, the biomolecule is selected from a protein, enzyme, or peptide, optionally wherein the biomolecule comprises at least one thiol group.

In some embodiments, Z is selected from

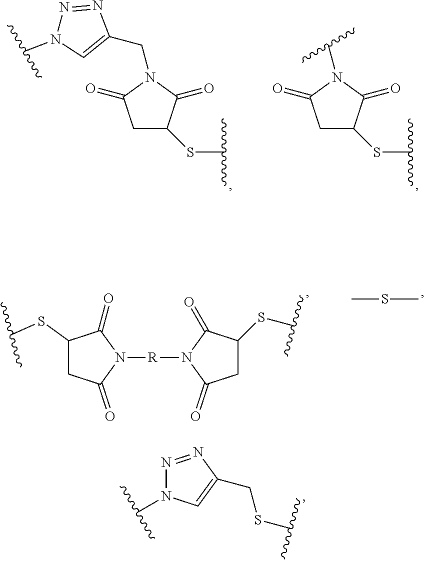

wherein R is selected from

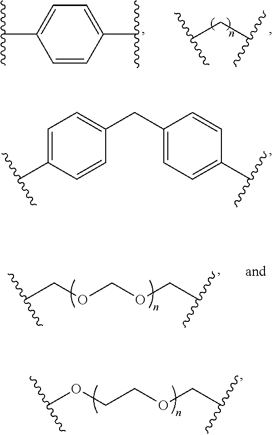

wherein is an integer from 1 to 5.

In one aspect, the disclosure provides a surface having a polymer of the disclosure or a compound of the disclosure grafted thereon. In some embodiments, the surface comprises a material selected from metals such as titanium and titanium alloys, iron, and steel; metal oxides; ceramics; polymers such as polyethylene (low and high reticulation for use in biomedical implants, after prior plasma activation), teflon (after prior plasma activation), polyethylene terephthalate (after prior plasma activation), and polypropylene (low and high density, after prior plasma activation), silicones, rubbers, latex, plastics, polyanhydrides, polyesters, polyorthoesters, polyamides, polyacrylonitrile, polyurethanes, polyethylene, polytetrafluoroethylene, polyethylenetetraphthalate and polyphazenes; paper; leather; textiles or textile materials such as cotton, jute, linen, hemp, wool, animals hair and silk, synthetic fabrics such as nylon and polyester; textile material comprising fibers comprising fiber material such as acrylic polymers, acrylate polymers, aramid polymers, cellulosic materials, cotton, nylon, polyolefins, polyester, polyamide, polypropylene, rayon, wool, spandex, silk, and viscose; silicon; wood; glass; cellulosic compounds; and gels and fluids not normally found within the human body.

In one aspect, the disclosure provides a method of controlling the growth of at least one bacteria, fungi, protozoa, or virus, the method comprising grafting a polymer of the disclosure or a compound of the disclosure onto a surface. In some embodiments, the bacteria is a gram-positive bacteria selected from M tuberculosis (including multi drug resistant TB and extensively drug resistant TB), M bovis, M typhimurium, M bovis strain BCG, BCG substrains, M avium, M intracellulare, M africanum, M kansasii, M marinum, M ulcerans, M avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthraces, B. subtilis, Nocardia asteroides, and other Nocardia species, Streptococcus viridans group, Peptococcus species, Peptostreptococcus species, Actinomyces israelii and other Actinomyces species, Propionibacterium acnes, Clostridium tetani, Clostridium perfringens, Clostridium botulinum, other Clostridium species, and Enterococcus species. In some embodiments, the bacteria is a gram-negative bacteria selected from Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species, Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, Escherichia hirae, and other Escherichia species, as well as other Enterobacteriacae, Burkholderia cepacia, Burkholderia pseudomallei, Francisella tularensis, Bacteroides fragilis, Fusobascterium nucleatum, Provetella species, Cowdria ruminantium, Klebsiella species, and Proteus species. In some embodiments, the virus is selected from influenza, Middle East respiratory syndrome-related coronavirus (MERS-CoV), rhinovirus, polio, measles, Ebola, Coxsackie, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, Eastern equine encephalitis, Western equine encephalitis, severe acute respiratory syndrome (SARS), severe acute respiratory syndrome coronavirus 2 (SARS-CoV-2), parainfluenza, Tacaribe, and Pichinde viruses.

In one aspect, the disclosure provides a coating comprising metal oxide nanoparticles and one or more polymers of the disclosure and/or one or more compounds of the disclosure. In some embodiments, a plurality of the metal oxide nanoparticles are substantially in contact with a surface. In some embodiments, the one or more polymers are grafted onto the surface of one or more of metal oxide nanoparticles. In some embodiments, the metal oxide nanoparticles comprise titanium oxide nanoparticles.

### BRIEF DESCRIPTION OF THE FIGURES

FIG. 1 illustrates a comparison between control and ODMcat-treated filter paper after sonicating and drying. The orange appearance of ODMcat-treated filter paper is due to the high number of counter ion of the fluorescein dye bound to the quaternary amino groups of the ODMcat moiety, which is covalently attached to the cotton.

FIG. 2 illustrates a comparison between control and ODMcat-treated cotton after vortexing, sonication and drying. The orange appearance of ODMcat-treated cotton is due to the extremely high number of counter ion of the fluorescein dye bound to the quaternary amino groups of the ODMcat moiety, which is covalently attached to the cotton.

FIG. 3 illustrates a comparison between control and C2-treated cotton after vortexing.

FIG. 4 illustrates a comparison between control and C2-treated cotton after vortexing, sonication and drying. The orange appearance of C2-treated cotton is due to the extremely high number of counter ion of the fluorescein dye bound to the quaternary amino groups of the C2 moiety, which is covalently attached to the cotton.

FIG. 5 illustrates a comparison between control and C2-treated filter paper after fluorescein test. The orange appearance of C2-treated filter paper is due to the extremely high number of fluorescein dye molecules bound to the quaternary amino groups of the C2 moiety, which is covalently attached to the cotton.

FIG. 6 illustrates the structure of a monomer of poly(vinylbenzyl chloride).

FIG. 7 illustrates the structure of a monomer of polyethylenimine.

FIG. 8 illustrates the structure of a fully methylated monomer of PEI.

FIG. 9 illustrates an exemplary synthesis to produce bis[3-(trimethoxysilyl)propyl-N,N′-tetraalkylethylenediammonium compounds. In some embodiments, bis[3-(trimethoxysilyl)propyl-N,N′-tetraalkylethylenediammonium compounds are both hydrophobic and hydrophilic.

FIG. 10. illustrates an exemplary synthesis of bis(3-trimethoxysilylpropyl)-N,N-methylalkylammonium bromide. In some embodiments, bis(3-trimethoxysilylpropyl)-N,N-methylalkylammonium compounds exhibit antimicrobial properties.

FIG. 11A illustrates an exemplary synthesis of bis(3-trimethoxysilylpropyl)dialkylammonium halide from bis(3-methoxysilylpropyl)amine and alkyl bromide (bromide may also be substituted for chloride or iodide). FIG. 11B illustrates an exemplary synthesis of bis(3-trimethoxysilylpropyl)difluoroalkylammonium bromide from bis(3-methoxysilylpropyl)amine and a perfluorinated alkyl bromide (bromide may also be substituted for chloride or iodide).

FIG. 12A illustrates an exemplary synthesis to prepare a polyvinylpyridine polymer comprising a monomer comprising a quaternary bis(3-trimethoxysilylpropyl)-N-methyl-N-paraxylyl moiety linked to the PVP polymer by a —CH₂-Ph-CH₂— linking group, and a monomer comprising a C₄-C₁₂ alkyl moiety (r=3-11), by reacting α,α′-Dibromo-p-xylene with bis(3-trimethyoxysilylpropyl)-N-methyamine, followed by treatment with polyvinylpyridine and then treatment with a C₄-C₂₂ alkyl halide, such as a C₄-C₂₂ alkyl bromide. x represents the molar ratio of the monomer comprising a quaternary bis(3-trimethoxysilylpropyl)-N-methyl-N-paraxylyl moiety, and (1-x) represents the molar ratio of the monomer comprising the alkylated quaternary pyridine moiety. FIG. 12B illustrates an exemplary synthesis to prepare a polymer comprising a monomer comprising a tertiary amine linked to a quaternary bis(3-trimethoxysilylpropyl)-N-methyl-N-paraxylyl moiety by a —C(CO)CH₂— linking group. x represents the molar ratio of the monomer comprising a quaternary bis(3-trimethoxysilylpropyl)-N-methyl-N-paraxylyl moiety, and (1-x) represents the molar ratio of the monomer comprising the quaternary trialkylamine moiety. FIG. 12C illustrates an exemplary synthesis to prepare a polyvinylpyridine polymer comprising a monomer comprising a quaternary bis(3-trimethoxysilylpropyl)-N-methyl-N-paraxylyl moiety linked to the PVP polymer by a —C(O)CH₂-linking group, and a monomer comprising a C₄-C₁₂ alkyl moiety (r=3-11), by reacting α,α′-Dibromo-p-xylene with bis(3-trimethoxysilylpropyl)-N-methylamine, followed by treatment with polyvinylpyridine and then treatment with a C₄-C₂₂ alkyl halide, such as a C₄-C₂₂ alkyl bromide. x represents the molar ratio of the monomer comprising a quaternary bis(3-trimethoxysilylpropyl)-N-methyl-N-paraxylyl moiety, and (1-x) represents the molar ratio of the monomer comprising the alkylated quaternary pyridine moiety. In a non-limiting embodiment, x is about 10, and 1-x is about 90.

FIG. 13 illustrates an exemplary synthesis of a polymer comprising a monomer comprising quaternary bis(3-trimethoxysilylpropyl)-N-methylalkylammonium moiety and a monomer comprising a quaternary dialkyl amine, such as (CH₃)₂N[(CH₂)ᵣCH₃], wherein r is 3-11.

FIG. 14 illustrates an IR spectrum of Octadecyl(4-catecholacetyl)dimethylammonium chloride. The bands at 2918 cm⁻¹ and 2852 cm⁻¹ correspond to the C—H stretching and show the successful introduction of the alkyl chain. 1684 cm⁻¹ bands are identical in the two spectra. They are due to the carbonyl aromatic stretching. The spectrum includes the comparative spectrum of 4-chloroacetylcatechol.

FIG. 15 illustrates an IR spectrum catecholacetyl-cobutyl PVP spectrum (blue line). The figure includes the comparative spectrum of 4-chloroacetylcatechol (pink line).

FIG. 16 illustrates an IR spectrum of poly(vinylbenzyl chloride) partially quaternized with bis(N-methyl)3propyltrimethoxysilane groups and N,N-dimethylbutyl groups compared to the commercial poly(vinylbenzyl chloride) (55 kda). The solvent is still visible on the 3378 cm⁻¹ band. The three bands corresponding to ν CH2,CH3 are the alkyl chains. Dotted line bands represent the ν CH2 of the benzyl groups in polyvinylbenzylchloride. The CH2, CH3 bands of the alkyl chains show the appropriate quaternization of the polymer.

FIG. 17 illustrates an IR spectrum of bis(3-trimethoxysilyl)propyl-N,N-dioctadecyl ammonium bromide. The three bands at 2970 cm⁻¹, 2921 cm⁻¹ and 2853 cm⁻¹ are due to the CH2, CH3 stretching. The bands at 1034 cm⁻¹ and 888 cm⁻¹ are the fingerprint of the methoxysilane moiety.

FIG. 18 illustrates an IR spectrum of the catecholacetyl-cobutyl PVP co-polymer with non-quaternized PVP in the background.

FIG. 19 illustrates an IR spectrum of the catecholacetyl-cobutyl PVP co-polymer. The bands at 2935 cm⁻¹ and 2871 cm⁻¹ show the C—H stretching in the butyl chain. The spectrum shows the intense bands due to the C—N⁺ stretching in the polymer (1634 cm⁻¹). The band at 1680 cm⁻¹ is due to the aromatic carbonyl of the catechol which corresponds to the shoulder at the same frequency in the catechol moiety incorporated in the polymer.

FIG. 20 illustrates an IR spectrum of the catecholacetyl-codecyl PVP co-polymer with non-quaternized PVP in the background. The three bands at 2954 cm⁻¹, 2923 cm⁻¹, and 2853 cm⁻¹ are due to the CH2, CH3 stretching. The 1639 cm⁻¹ band represents the C—N⁺ stretching. The 1678 cm⁻¹ band represents the aromatic nu C═O of the catechol moiety.

FIG. 21 illustrates an IR spectrum of the poly(vinylbenzyl chloride) co-polymer partially quaternized with bis(N-methyl)3propyltrimethoxysilane groups and N,N-dimethylbutyl groups. The solvent is still visible on the 3373 cm⁻¹ band. The three bands corresponding to ν CH₂,CH₃ are the alkyl chains. The band CH₂, CH₃ of the alkyl chains show the appropriate quaternization of the polymer.

FIG. 22 illustrates an IR spectrum of fully methylated quaternized PEI random copolymer partially grafted with acetylcatechol group and decyl group in ratio 1/9. The ν CH aromatic band is located at 3009 cm⁻¹. The three bands CH₃, CH₂ represent the alkyl chains. There is a slight shoulder at 1674 cm⁻¹ due to the aromatic carbonyl stretching. The 1633 cm⁻¹ band is due to the C—N⁺ stretching.

FIG. 23 illustrates IR spectra of methylated hyperbranched PEI (750 kda) and commercial PEI (750 kda). The disappearance of the 3277 cm⁻¹ band in the methylated PEI random copolymer proves that the methylation was near complete compared with polyethylenimine.

FIG. 24 illustrates an IR spectrum of a dipodal quaternized PVP, which is a partially quaternized PVP random copolymer with a quaternary bis(3-trimethoxysilylpropyl)-N-methyl-N-paraxylyl moiety and a butyl moiety in 1/9 ratio.

FIG. 25 illustrates an IR spectrum of bis(3-trimethoxysilypropyl)-N-bromoacetylamine.

FIG. 26 illustrates an IR spectrum of dipodal quaternized PVP, which is a partially quaternized PVP random copolymer with a quaternary bis(3-trimethoxysilylpropyl)-N-methyl-N-paraxylyl moiety and a butyl moiety in 1/9 ratio compared to the intermediate product bis(3-trimethoxysilypropyl)-N-bromoacetylamine. The 1639 cm⁻¹ band shows the quaternization of PVP. The bands at 1063 cm⁻¹ and 903 cm⁻¹, and 913 cm⁻¹ correspond to the methoxysilane moiety that are both present in the bis(3-trimethoxysilypropyl)-N-bromoacetylamine compound and the dipodal quaternized PVP.

FIG. 27 illustrates an IR spectrum of poly(vinylbenzyl chloride) partially quaternized with bis(N-methyl)3propyltrimethoxysilane groups and N,N-dimethylbutyl groups compared to the commercial poly(vinylbenzyl chloride) (55 kda). The solvent is still visible on the 3378 cm⁻¹ band. The three bands corresponding to ν CH₂,CH₃ are the alkyl chains. Dotted line bands represent the ν CH₂ of the benzyl groups in polyvinylbenzylchloride. The CH₂, CH₃ bands of the alkyl chains show the appropriate quaternization of the polymer.

FIG. 28A illustrates an exemplary scheme for the attachment of a biomolecule (e.g. protein, enzyme, peptide) containing a thiolated amino acid (e.g. R—SH) to a catechol moiety of the disclosure through azide-alkyne cycloaddition in two steps. In step A, the alkyne group is introduced in the biomolecule through a Michael addition. In step B, the biomolecule is attached to the catechol moiety through triazole formation between the alkyne and the azide groups. In step C, the catchol moiety can be grafted onto surfaces. FIG. 28B illustrates an exemplary scheme for the attachment of a biomolecule (e.g. protein, enzyme, peptide) containing a thiolated amino acid (e.g. R—SH) to a catechol moiety of the disclosure grafted to a surface through azide-alkyne cycloaddition in two steps.

FIG. 29 illustrates an exemplary scheme for the attachment of a biomolecule (e.g. protein, enzyme, peptide) containing a thiolated amino acid (e.g. R—SH) to a catechol moiety of the disclosure through a Michael addition. In step A, the maleimide is introduced in the catechol moiety. In step B, the biomolecule is attached to the maleimide through a Michael addition.

FIG. 30 illustrates an exemplary scheme for the attachment of a biomolecule (e.g. protein, enzyme, peptide) containing a thiolated amino acid (e.g. R—SH) to a catechol moiety through nucleophilic substitution. The thiol group of the biomolecule displaces the iodide group of the catechol moiety under buffered conditions to provide the conjugated product. A non-limiting example of buffered conditions include maintaining reaction conditions at a pH of about 8.3 using a borate buffer.

FIG. 31 illustrates an exemplary scheme for the attachment of a biomolecule (e.g. protein, enzyme, peptide) containing a thiolated amino acid (e.g. R—SH) to a catechol moiety comprising two maleimide moieties, wherein the maleimide moieties are conjugated to each other by way of a linker R. The thiol group of the catechol moiety is first conjugated to one of the maleimide moieties, and then the biomolecule is conjugated to the second maleimie moiety by way of a thiol group. Non-limiting examples of the linker R are shown.

FIG. 32 illustrates a non-limiting example of a method of preparing a biocidal coating of the disclosure.

FIG. 33 illustrates an IR spectrum of bis[3-(trimethoxysilyl)propyl-N,N′-tetraalkylethylenediammonium bromide. The three bands at 2950 cm⁻¹, 2921 cm⁻¹ and 2851 cm⁻¹ are due to the CH₂, CH₃ stretching. The bands at 1040 cm⁻¹ and 886 cm⁻¹ are the fingerprint of the methoxysilane moiety.

FIG. 34 illustrates an IR spectrum of fully methylated quaternized PEI random copolymer partially grafted with propyltrimethoxysilane group and a hexyl group in ratio 1/9. The 1631 cm-band represents the C—N⁺ stretching. The CH₂, CH₃ stretching bands are seen at 2956 cm⁻¹, 2927 cm⁻¹, and 2859 cm⁻¹.

FIG. 35 illustrates a comparison between control and L-cysteine-grafted filter paper using 4-iodoacetylcatechol as a linker after 1% aqueous ninhydrin treatment. The treated filter paper appears purple, demonstrating the successful grafting of L-cysteine.

FIG. 36 illustrates a comparison between control and L-cysteine-grafted glass slides using 4-iodoacetylcatechol as a linker after 1% aqueous ninhydrin treatment. The treated glass slides displays multiple blue/purple spots, demonstrating the successful grafting of L-cysteine.

FIG. 37 illustrates a comparison between control filter paper and treated filter paper with quaternized bis(3-trimethoxysilylpropyl)-N-methyl-N-paraxylyl-cobutyl-PVP. The orange appearance of the filter paper treated with bis(3-trimethoxysilylpropyl)-N-methyl-N-paraxylyl-cobutyl-PVP is due to the high number of fluorescein dye molecules bound to the quaternary ammonium compounds. The polymer is covalently grafted to the filter paper (remained on the filter paper despite sonication).

FIG. 38 illustrates a comparison between control filter paper and treated filter paper with poly(vinylbenzyl chloride) partially quaternized with bis(N-methyl)₃-propyltrimethoxysilane groups and N,N-dimethylbutyl groups. The orange appearance of the filter paper treated with poly(vinylbenzyl chloride) partially quaternized with bis(N-methyl)₃-propyltrimethoxysilane groups and N,N-dimethylbutyl groups is due to the high number of fluorescein dye molecules bound to the quaternary ammonium compounds. The polymer is covalently grafted to the filter paper (remained on the filter paper despite sonication).

FIG. 39 illustrates a comparison between control filter paper and treated filter paper with 3-trimethoxypropyl-cohexyl-PEI (from PEI at 750 kda). The orange appearance of the filter paper treated with 3-trimethoxypropyl-cohexyl-PEI is due to the high number of fluorescein dye molecules bound to the quaternary ammonium compounds. The polymer is covalently grafted to the filter paper (remained on the filter paper despite sonication).

FIG. 40 illustrates a comparison between control filter paper and treated filter paper with 3-trimethoxypropyl-codecyl-PEI (from PEI at 25 kda). The orange appearance of the filter paper treated with 3-trimethoxypropyl-codecyl-PEI is due to the relatively high number of fluorescein dye molecules bound to the quaternary ammonium compounds. The polymer is covalently grafted to the filter paper (remained on the filter paper despite sonication).

FIG. 41 illustrates a comparison between control filter paper and treated filter paper with bis(3-trimethoxysilypropyl)-N-bromoacetylamine. The orange appearance of the filter paper treated with bis(3-trimethoxysilypropyl)-N-bromoacetylamine is due to the high number of fluorescein dye molecules bound to the quaternary ammonium compounds. The compound is covalently grafted to the filter paper (remained on the filter paper despite sonication).

FIG. 42 illustrates a comparison between control filter paper and treated filter paper with bis(3-trimethoxysilyl)propyl-N,N-dihexadecyl ammonium bromide. The orange appearance of the filter paper treated with bis(3-trimethoxysilyl)propyl-N,N-dihexadecyl ammonium bromide is due to the relatively high number of fluorescein dye molecules bound to the quaternary amino groups of the quaternary ammonium compounds. The monomer is covalently attached to the filter paper (remained on the filter paper despite sonication). The orange color sometimes appears slightly more intense for monomers than polymers with filter paper due to the fact that fluorescein penetrates deeper into the paper when only monomers are grafted. This is not related to the charge density.

FIG. 43 illustrates a comparison between control filter paper and treated filter paper with bis(3-trimethoxysilylpropyl)-N,N-methylalkylammonium bromide. The orange appearance of the filter paper treated with bis(3-trimethoxysilylpropyl)-N,N-methylalkylammonium bromide is due to the relatively high number of fluorescein dye molecules bound to the quaternary ammonium compounds. The monomer is covalently attached to the filter paper (remained on the filter paper despite sonication). The orange color sometimes appears slightly more intense for monomers than polymers with filter paper due to the fact that fluorescein penetrates deeper into the paper when only monomers are grafted. This is not related to the charge density.

FIG. 44 illustrates a comparison between control filter paper and treated filter paper with bis[3-(trimethoxysilyl)propyl-N,N′-tetraalkylethylenediammonium. The orange appearance of the filter paper treated with bis[3-(trimethoxysilyl)propyl-N,N′-tetraalkylethylenediammonium is due to the relatively high number of fluorescein dye molecules bound to the quaternary ammonium compounds. The monomer is covalently attached to the filter paper (remained on the filter paper despite sonication). The orange color sometimes appears slightly more intense for monomers than polymers with filter paper due to the fact that fluorescein penetrates deeper into the paper when only monomers are grafted. This is not related to the charge density.

### DETAILED DESCRIPTION

Unless defined otherwise, all technical and scientific terms used herein have the same meaning as is commonly understood by one of skill in the art to which this invention belongs. All patents and publications referred to herein are incorporated by reference in their entireties.

### Definitions

The term “biocide”, as used herein, means a chemical compound, a chemical composition, a chemical formulation which can kill or render harmless a microorganism exemplified by bacterium, yeast, protozoa, and fungi.

The term “statistical copolymer” as used herein is defined as a copolymer that is made up of more than one monomer, and in which the different monomer units are randomly distributed in the polymeric chain.

As used herein, the terms “graft” and “grafting” refer to the attachment of moieties onto a surface by forming covalent linkages between functional groups on the surface and the moiety.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R² of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Oxa” refers to the —O— radical.

“Oxo” refers to the ═O radical.

“Moiety” refers to a specific segment or functional group of a molecule. Chemical moieties are often recognized chemical entities embedded in or appended to a molecule.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Compounds, Polymers and Compositions

The catechol moiety is known for ensuring covalent bonds with the majority of hydroxylated substrates, and has been utilized as a linker. However, the use of catecholamine has several drawbacks, especially its rapid polymerization at a basic pH and spontaneous oxidation to quinones.

As depicted in reaction sequence A of Scheme 1 below, reactions between a surface and a volume (eg. a solution comprising the bromide compound) usually occur according to an S_{N}² mechanism with the nucleophile almost always localized on the surface while the electrophile is located in the volume.

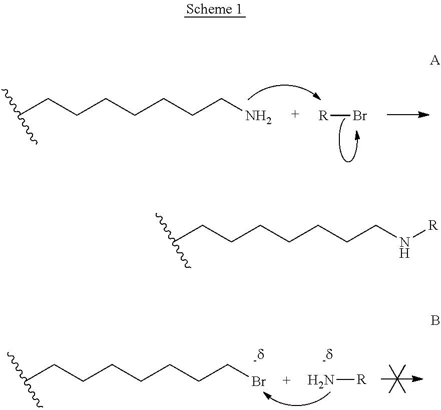

In contrast, the reaction sequence B of Scheme 1 cannot be completed due to electrostatic repulsions. The only super nucleophiles capable of displacing Br are the N₃⁻ and thiocyanate (SCN⁻) groups. Using compositions and methods described herein, the reaction sequence B becomes possible due to the particular reactivity of the C—Cl bond because of the enhanced reactivity of the C—Cl bond due to the presence of the electron withdrawing group (CO) in the alpha position.

The disclosure includes a series of novel compounds useful for grafting a large variety of compositions, such as polymers, onto a variety of surfaces. The disclosure also includes a series of novel catechol compounds useful for grafting a large variety of compositions, such as biomolecules and polymers including antimicrobial polymers and biomacromolecules, onto a variety of surfaces. In some embodiments, the compounds are derived from the catechol family, and are more stable than catecholamine which exhibits a propensity to polymerize.

In one aspect, the disclosure also includes the use of novel dipodal silane compounds. In some embodiments, the dipodal silane compounds are further substituted with an alkyl chain following treatment with an alkyl halide in a single step reaction to provide highly hydrophobic/hydrophilic compounds that are ready-to graft on a variety of surfaces, such as hydroxylated or activated surfaces, with the benefit of being much more stable and extremely resistant to hydrolysis compared to the conventional silanes. See U.S. Pat. No. 9,029,491, US 20050187400, U.S. Pat. Nos. 8,475,782, and 9,289,534, all of which are incorporated by reference herein in their entireties.

The disclosure also includes novel ready-to-graft biocidal polymers and compounds that can be covalently attached to surfaces. These polymers and compounds can be used in biocidal and antimicrobial compositions that are useful to combat healthcare-acquired infections (HAI) and virtually any type of environmental surface treatment. The biocidal polymers and compounds of the disclosure can be used to contain and control the spread of infectious pathogens in a variety of health and industrial applications.

Substrates, such as nanoparticles, antibodies, enzymes, and compositions and polymers comprising the moieties of the disclosure are easily graftable and provide bonds with improved stability and less sensitivity to hydrolysis than other moieties, such as silane linkers.

In one aspect of the disclosure, the polymers are prepared by covalently linking the chemical moieties to the polymer to produce polymers that can be easily grafted onto a variety of surfaces, including metal and wood. In one aspect of the disclosure, graftable substrates, including polymers, antibodies, enzymes, and peptides, are prepared by covalently linking the compounds of the disclosure to a substrate that can be easily grafted onto a variety of surfaces, including metal and wood. In some embodiments, the compounds of the disclosure are easily graftable to surfaces. In some embodiments, a ready-to-graft solution of the compounds of the invention is prepared in a one-pot synthesis. In some embodiments, the graftable substrates of the disclosure are prepared in a one-pot synthesis. In some embodiments, the polymers are prepared in a one-pot synthesis. In another aspect of the disclosure, compounds described herein can be easily grafted onto surfaces. When grafted, functional groups on the chemical moieties and compounds of the disclosure form covalent bonds with functional groups on the surface.

Over the past decade, there has been a tremendous need for self-cleaning surfaces that was exacerbated following the advent of the COVID-19 pandemic. Indeed, transient solutions such as disinfecting wipes are labor-intensive, costly and not sustainable in the long term. In order to address the shortcomings of existing temporary solutions, numerous surface coating strategies were developed to confer long-lasting antimicrobial properties to environmental surfaces.

While metal-ion-based coatings have been shown to be effective, they raise concerns of durability, toxicity and sustainability. In contrast, quaternary ammonium compounds (QAC) have long been known as potent and stable antimicrobial products when used as surface coatings. In the category of QAC, polymers were shown to be superior to monomers (classically C₁₈ quaternized alkyl chain such as 3-(trihydroxysilyl) propyldimethyloctadecyl ammonium chloride). The latter class of molecules was described in the early 1970s and sold by Dow-Corning from the late 1970s. The main reason for the low effectiveness of quaternary ammonium monomers is likely due to a surface charge density difference between monomers and polymers. Indeed, quaternary ammonium polymers can be turned into high-density QACs, exceeding 10¹⁵ charges/cm², which is the most commonly described threshold to achieve biocidal activity of surfaces. These compounds kill bacteria, viruses, and fungi even as a monolayer, provided that the charge density threshold is reached. This is not the case with simple quaternary alkyl-ammonium compounds.

While numerous products are currently marketed as spray-on coatings, their active ingredient is almost always 3-(trihydroxysilyl) propyldimethyloctadecyl ammonium chloride or any similar compound that is a C₁₈ quaternized alkyl chain. It is now well established in the literature that these compounds are poorly effective. On a monolayer, they typically display only a bacteriostatic effect due to their low charge density. In order to overcome their weak efficiency on surfaces, they are often used in volume such as in blending techniques which typically ensure that the compounds are found throughout the entire thickness of the substrate. Coatings with thick multilayers of C₁₈ quaternized alkyl chains can kill bacteria due to their increased charge densities. In contrast, biocidal polymers typically kill bacteria, viruses, and fungi regardless of the coating thickness due to their spatial conformation as a polymer brush.

### Compounds

In one aspect, the disclosure provides a compound of formula (Ib):

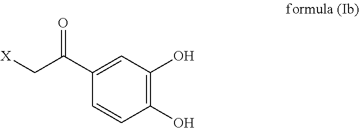

wherein in formula (Ib):
X is halogen, optionally substituted amine, azido, or C(O)OR⁴; SR⁴, and
R⁴ is selected from hydrogen and optionally substituted alkyl.

In some embodiments, the compound of formula (Ib) is selected from:

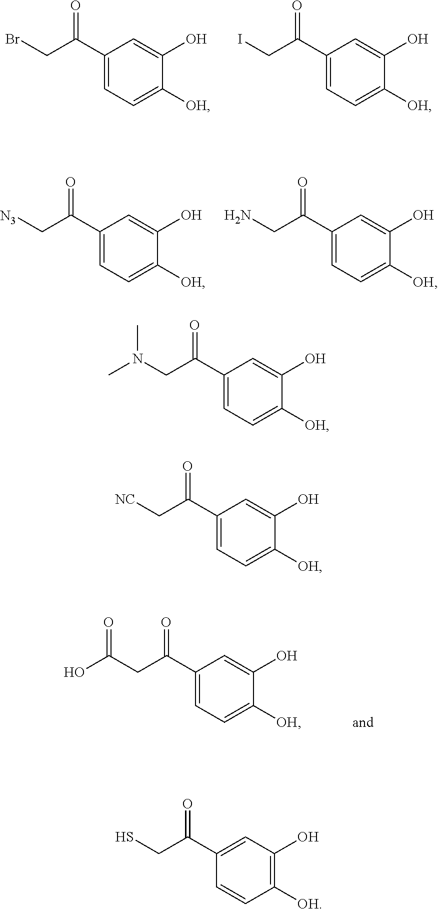

In another aspect, the disclosure describes a compound of formula (XV):

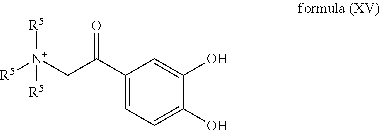

wherein in formula (XV):
each R⁵ is independently optionally substituted alkyl.

In some embodiments, each R⁵ is independently C₄-C₂₂ alkyl. In some embodiments, each R⁵ is independently C₁-C₂₂ alkyl.

In some embodiments, the compound of formula (XV) is a compound of formula (XVI):

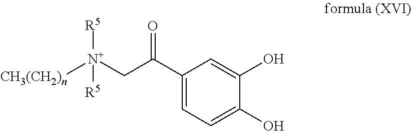

wherein in formula (XVI):
each R⁵ is independently optionally substituted alkyl; and
n is an integer from 3 to 21.

In some embodiments, n is an integer from 15 to 19. In some embodiments, n is 17. In some embodiments, each R⁵ is methyl.

In some embodiments, the compound is a compound of formula (XVa):

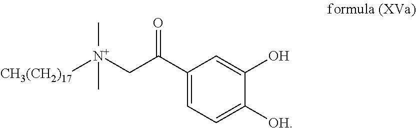

In another aspect, the disclosure describes a compound of formula (XVII):

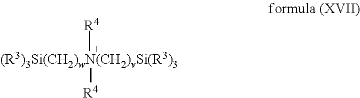

wherein in formula (XVII):
each R³ is independently optionally substituted alkoxy;
each R⁴ is independently optionally substituted alkyl, optionally substituted aryl, optionally substituted alkenyl, or optionally substituted alkynyl;
v is an integer from 3 to 10; and
w is an integer from 3 to 10.

In some embodiments, w is 3. In some embodiments, v is 3. In some embodiments, each R³ is independently optionally substituted methoxy. In some embodiments, each R³ is methoxy. In some embodiments, each R⁴ is independently C₁-C₂₂ alkyl or C₁-C₂₂ haloalkyl. In some embodiments, one R⁴ is methyl and one R⁴ is independently C₁-C₂₂ alkyl or C₁-C₂₂ haloalkyl. In some embodiments, each R⁴ is independently selected from —CH₂(CH₂)ₙCH₃ and —CH₂CH₂(CF₂)ₙCF₃, wherein n is an integer from 15 to 20. In some embodiments, each R⁴ is independently selected from —CH₂(CH₂)ₙCH₃ wherein n is an integer from 14 to 20. In some embodiments, each R⁴—CH₂(CH₂)₁₄CH₃. In some embodiments, each R⁴—CH₂(CH₂)₁₆CH₃. In some embodiments, each R⁴ is independently selected from —CH₂(CH₂)ₙCH₃ wherein n is 14, 16, 18, or 20. In some embodiments, each R⁴ is independently selected from —CH₂CH₂(CF₂)ₙCF₃ wherein n is an integer from 15 to 19. In some embodiments, each R⁴ is independently selected from —CH₂CH₂CH₂(CF₂)ₙCF₃ wherein n is 15, 17, or 19.

In another aspect, the disclosure describes a compound of formula (XVIIa):

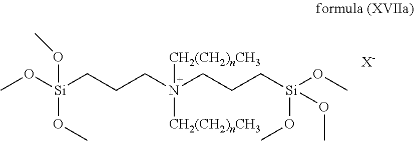

wherein in formula (XVIIa):
n is an integer between 16 and 20; and
X is Br, Cl, or I.

In some embodiments, the compound of formula (XVIIa) is:

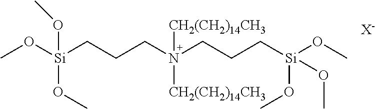

wherein X is a counterion. In some embodiments, X is Br.

In some embodiments, the compound of formula (XVIIa) is:

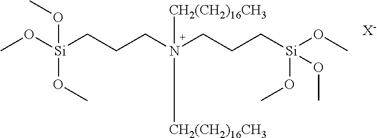

wherein X is a counterion. In some embodiments, X is Br.

In another aspect, the disclosure describes a compound of formula (XVIIb):

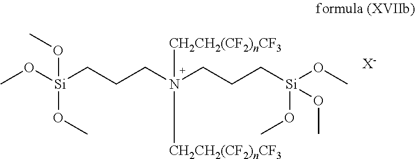

wherein in formula (XVIIb):
n is an integer between 15 and 19; and
X is Br, Cl, or I.

In another aspect, the disclosure describes a compound of formula (XVIIc):

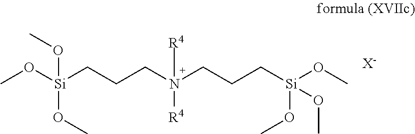

wherein in formula (XVIIc):
each R⁴ is independently selected from optionally substituted alkyl, optionally substituted aryl, optionally substituted alkenyl, and optionally substituted alkynyl;
X is Br, Cl, or I. In some embodiments, each R⁴ is independently optionally substituted C₁₈-C₂₂ alkyl. In some embodiments, each R⁴—CH₂(CH₂)₁₄CH₃. In some embodiments, each R⁴—CH₂(CH₂)₁₆CH₃.

In another aspect, the disclosure describes a compound of formula (XVIII):

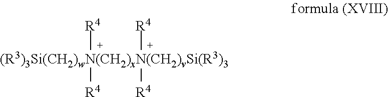

wherein in formula (XVIII):
each R³ is independently optionally substituted alkoxy;
each R⁴ is independently optionally substituted alkyl, optionally substituted aryl, optionally substituted alkenyl, or optionally substituted alkynyl; v is an integer from 3 to 10;
w is an integer from 3 to 10; and
x is an integer from 1 to 4.

In some embodiments, w is 3. In some embodiments, v is 3. In some embodiments, x is 2. In some embodiments, each R³ is independently optionally substituted methoxy. In some embodiments, each R³ is methoxy. In some embodiments, each R⁴ is independently C₁₈ alkyl or C₁₈ haloalkyl. In some embodiments, each R⁴ is independently C₁₆ alkyl or C₁₆ haloalkyl. In some embodiments, each R⁴ is independently C₁-C₂₂ alkyl or C₁-C₂₂ haloalkyl. In some embodiments, two R⁴ are methyl and two R⁴ is independently C₁-C₂₂ alkyl or C₁-C₂₂ haloalkyl. In some embodiments, each R⁴ is independently selected from —CH₂(CH₂)ₙCH₃ and —CH₂(CF₂)ₙCF₃, wherein n is an integer from 14 to 20. In some embodiments, each R⁴ is independently selected from —CH₂(CH₂)ₙCH₃ wherein n is an integer from 14 to 20. In some embodiments, each R⁴ is independently selected from —CH₂(CH₂)ₙCH₃ wherein n is 14, 16, 18, or 20. In some embodiments, each R⁴ is independently selected from —CH₂CH₂CH₂(CF₂)ₙCF₃ wherein n is an integer from 15 to 19. In some embodiments, each R⁴ is independently selected from —CH₂CH₂CH₂(CF₂)ₙCF₃ wherein n is 15, 17, or 19.

In some embodiments, the compound of formula (XVIII) is a compound of formula (XVIIIa):

In another aspect, the disclosure describes a compound of formula (XIX):

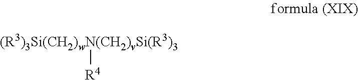

wherein in formula (XIX):
each R³ is independently optionally substituted alkoxy;
R⁴ is independently optionally substituted alkyl, optionally substituted aryl, optionally substituted alkenyl, or optionally substituted alkynyl;
v is an integer from 3 to 10; and
w is an integer from 3 to 10.

In some embodiments, w is 3. In some embodiments, v is 3. In some embodiments, each R³ is independently optionally substituted methoxy. In some embodiments, In some embodiments, each R³ is methoxy. In some embodiments, R⁴ is C₁-C₂₂ alkyl or C₁-C₂₂ haloalkyl. In some embodiments, R⁴ is C₁₈ alkyl or C₁₈ haloalkyl. In some embodiments, each R⁴ is independently C₁₆ alkyl or C₁₆ haloalkyl. In some embodiments, R⁴ is selected from —CH₂(CH₂)ₙCH₃ and —CH₂CH₂(CF₂)ₙCF₃, wherein n is an integer from 15 to 20. In some embodiments, R⁴ is —CH₂(CH₂)ₙCH₃ wherein n is an integer from 14 to 20. In some embodiments, R⁴ is —CH₂(CH₂)ₙCH₃ wherein n is 14, 16, 18, or 20. In some embodiments, n is 14. In some embodiments, n is 16. In some embodiments, R⁴ is —CH₂CH₂(CF₂)ₙCF₃ wherein n is an integer from 15 to 19. In some embodiments, R⁴ is —CH₂CH₂(CF₂)ₙCF₃ wherein n is 15, 17, or 19. In some embodiments, n is 15. In some embodiments, n is 17. In some embodiments, R⁴ is selected from

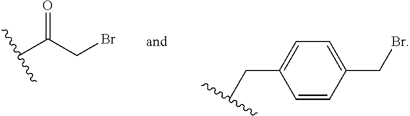 [SMILES: *C(=O)CBr ; *Cc1ccc(CBr)cc1]

In another aspect, the disclosure describes a compound of formula (XIXa):

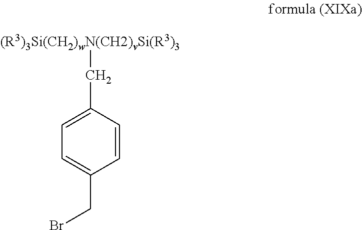

wherein in formula (XIXa):
each R³ is independently optionally substituted alkoxy;
v is an integer from 3 to 10; and
w is an integer from 3 to 10.

In some embodiments, w is 3. In some embodiments, v is 3. In some embodiments, each R³ is independently optionally substituted methoxy. In some embodiments, In some embodiments, each R³ is methoxy.

In another aspect, the disclosure describes a compound of formula (XIXb):

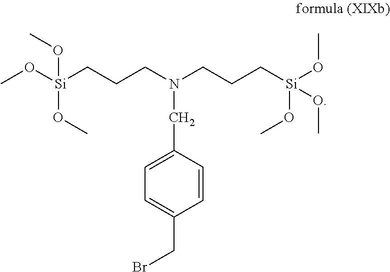

In another aspect, the disclosure describes a compound of formula (XIXc):

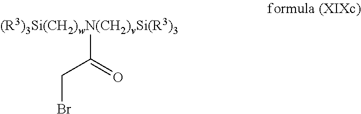

wherein in formula (XIXc):
each R³ is independently optionally substituted alkoxy;
v is an integer from 3 to 10; and
w is an integer from 3 to 10.

In some embodiments, w is 3. In some embodiments, v is 3. In some embodiments, each R³ is independently optionally substituted methoxy. In some embodiments, In some embodiments, each R³ is methoxy.

In another aspect, the disclosure describes a compound of formula (XIXd):

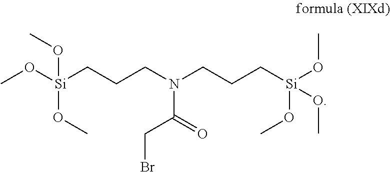

In another aspect, the disclosure describes a compound of formula (XX):

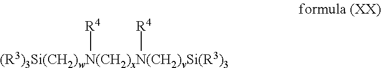

wherein in formula (XX):
each R³ is independently optionally substituted alkoxy;
each R⁴ is independently optionally substituted alkyl, optionally substituted aryl, optionally substituted alkenyl, or optionally substituted alkynyl;
v is an integer from 3 to 10;
w is an integer from 3 to 10; and
x is an integer from 1 to 4.

In some embodiments, w is 3. In some embodiments, v is 3. In some embodiments, x is 2. In some embodiments, each R³ is independently optionally substituted methoxy. In some embodiments, each R³ is methoxy. In some embodiments, each R⁴ is independently C₁-C₂₂ alkyl or C₁-C₂₂ haloalkyl. In some embodiments, each R⁴ is independently C₁₈ alkyl or C₁₈ haloalkyl. In some embodiments, each R⁴ is independently C₁₆ alkyl or C₁₆ haloalkyl. In some embodiments, two R⁴ are methyl and two R⁴ are independently C₁-C₂₂ alkyl or C₁-C₂₂ haloalkyl. In some embodiments, each R⁴ is independently selected from —CH₂(CH₂)ₙCH₃ and —CH₂CH₂CH₂(CF₂)ₙCF₃, wherein n is an integer from 15 to 20. In some embodiments, each R⁴ is independently selected from —CH₂(CH₂)ₙCH₃ wherein n is an integer from 14 to 20. In some embodiments, each R⁴ is independently selected from —CH₂(CH₂)ₙCH₃ wherein n is 14, 16, 18, or 20. In some embodiments, n is 14. In some embodiments, n is 16. In some embodiments, each R⁴ is independently selected from —CH₂CH₂(CF₂)ₙCF₃ wherein n is an integer from 15 to 19. In some embodiments, n is 16. In some embodiments, each R⁴ is independently selected from —CH₂CH₂(CF₂)ₙCF₃ wherein n is 15, 17, or 19. In some embodiments, n is 15. In some embodiments, n is 17.

In another aspect, the disclosure describes a compound of formula (XXa):

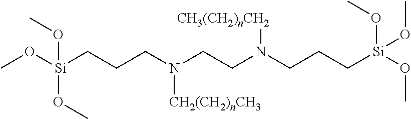

wherein in formula (XXa):
n is an integer from 16-20.

In one aspect, the disclosure describes compositions comprising at least one compound of the disclosure. In some embodiments, the compound is any one of a compound of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa). In some embodiments, the composition is an antibacterial composition. In some embodiments, the composition is a biocidal composition. In some embodiments, the composition is an antiviral composition. In some embodiments, the composition is an antifungal composition. In some embodiments, the composition is an antiprotozoal composition.

In one aspect, the disclosure describes a solution comprising an alcohol and at least one compound of the disclosure. In some embodiments, the compound is any one of a compound of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa). Any alcohol can be used, as understood by one of ordinary skill in the art. Non-limiting examples of alcohols include ethanol, methanol, n-propanol, isopropanol, t-butyl alcohol, and t-amyl alcohol. In some embodiments, the solution is an antibacterial solution. In some embodiments, the solution is a biocidal solution. In some embodiments, the solution is an antiviral solution. In some embodiments, the solution is an antifungal solution. In some embodiments, the solution is an antiprotozoal solution. In some embodiments, the solution is a ready-to-use solution for grafting.

### Graftable Compounds and Substrates

In one aspect, the disclosure provides a graftable substrate comprising compounds and/or moieties of the invention. In some embodiments, the graftable substrate comprises at least one compound of any one of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa). In some embodiments, the graftable substrate comprises at least one moiety of any one of formula (I) or formula (Ia). In some embodiments, the compound of any one of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa) is grafted onto a substrate.

As would be understood by one of ordinary skill in the art, any substrate is contemplated by the disclosure. Non-limiting examples of substrates include polymers, antibodies, enzymes, peptides, and proteins.

In one aspect of the disclosure, the graftable substrate comprises at least one moiety of formula (I):

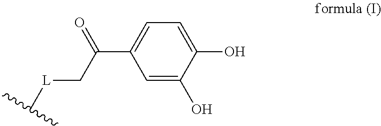

wherein in formula (I):
L is a single bond or a linking group.

In some embodiments, L is a single bond. In some embodiments, L is a linking group. The linking group may be any organic moiety, as would be understood by one of ordinary skill in the art. In some embodiments, the linking group comprises optionally substituted amino, optionally substituted heterocyclyl, carboxyl, or optionally substituted thiol. In some embodiments, the linking group comprises a primary amino group, a secondary amino group, or a tertiary amino group. In some embodiments, the organic linker comprises dimethylamino, diethylamino, —C(O)O—, —S—, diethylcarboxylate, acetyl, optionally substituted triazole group, or optionally substituted tetrazole group.

In some embodiments, the moiety of formula (I) is a moiety of formula (Ia):

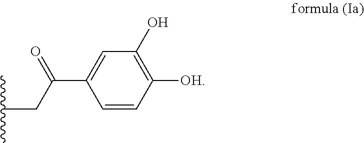 [SMILES: *CC(=O)c1ccc(O)c(O)c1]

In another aspect, the graftable substrate comprises at least one moiety of formula (XVIa):

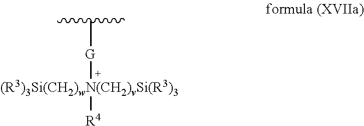

wherein in formula (XVIIa):
G is a single bond or a linking group;
each R³ is independently optionally substituted alkoxy;
each R⁴ is independently optionally substituted alkyl;
v is an integer from 3 to 10; and
w is an integer from 3 to 10.

In another aspect, the graftable substrate comprises at least one moiety of formula (XVIIIa):

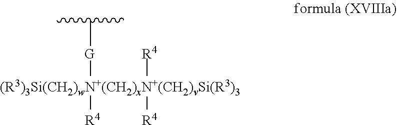

wherein in formula (XVIIIa):
G is a single bond or a linking group;
each R³ is independently optionally substituted alkoxy;
each R⁴ is independently optionally substituted alkyl;
v is an integer from 3 to 10;
w is an integer from 3 to 10; and
x is an integer from 1 to 4.

In some embodiments, G is a single bond. In some embodiments, G is a linking group. The linking group G may be any organic moiety, as would be understood by one of ordinary skill in the art. In some embodiments, the linking group comprises

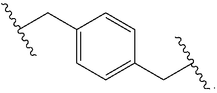 [SMILES: *Cc1ccc(C*)cc1]

In some embodiments, the linking group comprises

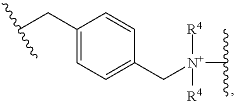

wherein each R⁴ is independently optionally substituted alkyl. In some embodiments, each R⁴ is independently selected from C₁-C₄ alkyl and

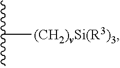

wherein each R³ is independently optionally substituted alkoxy, and v is an integer from 3 to 10. In some embodiments, each R⁴ is independently selected from methyl and

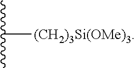

In some embodiments, one R⁴ is methyl and one R⁴ is

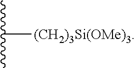

In some embodiments, w is 3. In some embodiments, v is 3. In some embodiments, x is 2. In some embodiments, each R³ is independently optionally substituted methoxy. In some embodiments, each R³ is methoxy. In some embodiments, each R⁴ is independently C₁-C₂₂ alkyl or C₁-C₂₂ haloalkyl. In some embodiments, each R⁴ is independently C₁₈ alkyl or C₁₈ haloalkyl. In some embodiments, each R⁴ is independently C₁₆ alkyl or C₁₆ haloalkyl. In some embodiments, two R⁴ are methyl and two R⁴ is independently C₁-C₂₂ alkyl or C₁-C₂₂ haloalkyl. In some embodiments, each R⁴ is independently selected from —CH₂(CH₂)ₙCH₃ and —CH₂CH₂(CF₂)ₙCF₃, wherein n is an integer from 14 to 20. In some embodiments, each R⁴ is independently selected from —CH₂(CH₂)ₙCH₃ wherein n is an integer from 14 to 20. In some embodiments, each R⁴ is independently selected from —CH₂(CH₂)ₙCH₃ wherein n is 14, 16, 18, or 20. In some embodiments, n is 14. In some embodiments, n is 16. In some embodiments, each R⁴ is independently selected from —CH₂CH₂(CF₂)ₙCF₃ wherein n is an integer from 15 to 19. In some embodiments, each R⁴ is independently selected from —CH₂CH₂(CF₂)ₙCF₃ wherein n is 15, 17, or 19. In some embodiments, n is 15. In some embodiments, n is 17.

In one aspect, the disclosure describes compositions comprising at least one graftable substrate of the disclosure. In some embodiments, the composition is an antibacterial composition. In some embodiments, the composition is a biocidal composition. In some embodiments, the composition is an antiviral composition. In some embodiments, the composition is an antifungal composition. In some embodiments, the composition is an antiprotozoal composition.

In one aspect, the disclosure describes a solution comprising an alcohol and at least one graftable substrate of the disclosure. Any alcohol can be used, as understood by one of ordinary skill in the art. Non-limiting examples of alcohols include ethanol, methanol, n-propanol, isopropanol, t-butyl alcohol, and t-amyl alcohol. In some embodiments, the solution is an antibacterial solution. In some embodiments, the solution is a biocidal solution. In some embodiments, the solution is an antiviral solution. In some embodiments, the solution is an antifungal solution. In some embodiments, the solution is an antiprotozoal solution. In some embodiments, the solution is a ready-to-use solution for grafting.

In one aspect, the disclosure provides methods for preparing a graftable substrate. In some embodiments, the disclosure includes methods for preparing a graftable substrate comprising a compound of any one of formula (XV), formula (XVI), or formula (XVa). In some embodiments, the disclosure includes methods for preparing a graftable substrate comprising a moiety of any one of formula (I) or formula (Ia).

In some embodiments, the method includes treating a substrate with a compound of formula (Ib):

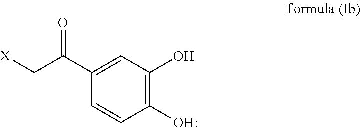

wherein in formula (Ib):
X is halogen, optionally substituted amine, azido, cyano, —C(O)OR⁴; or —SR⁴, and
R⁴ is selected from hydrogen and optionally substituted alkyl.

In some embodiments, the compound of formula (Ib) is selected from:

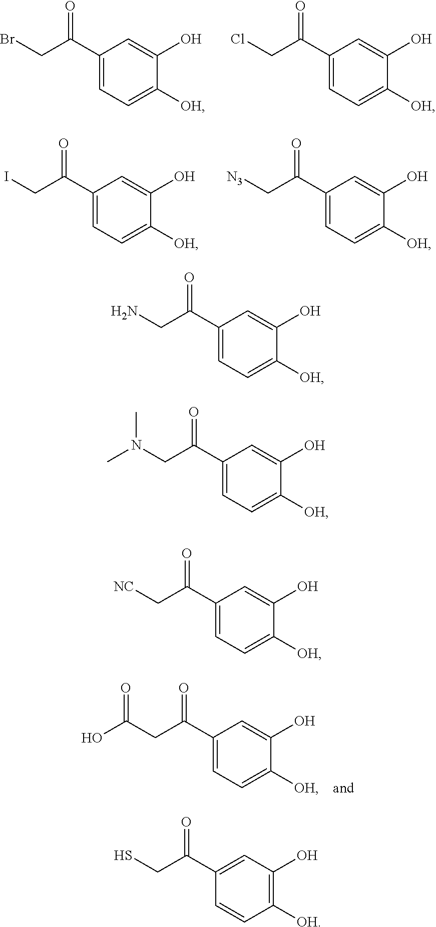

In some embodiments, the method includes treating a substrate with one or more compounds of any one of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa).

In some embodiments, the substrate is selected from a polymer, an antibody, an enzyme, a peptide, and a protein. In some embodiments, the precursor polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide), poly(diallyldimethylammonium), or a C₃-C₂₂ alkyne. In some embodiments, the method further includes treating the precursor polymer with the compound of formula (Ib) in a solvent selected from ethanol, methanol, n-propanol, isopropanol, t-butyl alcohol, and t-amyl alcohol. In some embodiments, the method further includes treating the precursor polymer with at least one optionally substituted C₄-C₂₂ alkyl halide.

### Polymers

In one aspect, the disclosure provides polymers comprising a compound of the disclosure. In one aspect, the disclosure provides polymers comprising a moiety of the disclosure.

In one aspect, the disclosure describes a polymer comprising at least one moiety of formula (I):

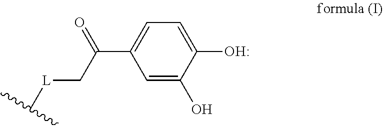

wherein in formula (I):
L is a single bond or a linking group.

In some embodiments, L is a single bond. In some embodiments, L is a linking group. The linking group may be any organic moiety, as would be understood by one of ordinary skill in the art. In some embodiments, the linking group comprises optionally substituted amino, optionally substituted heterocycle, optionally substituted thiol, or carboxylate. In some embodiments, the linking group comprises a primary amino group, a secondary amino group, or a tertiary amino group. In some embodiments, the organic linker comprises dimethylamino, diethylamino, —C(O)O—, —S—, diethylcarboxylate, acetyl, optionally substituted triazole group, optionally substituted maleic an or optionally substituted tetrazole group.

Any polymer is contemplated for use within the disclosure, as would be understood by one of ordinary skill in the art. In some embodiments, the polymer is a random copolymer. In some embodiments, the polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide) and poly(diallyldimethylammonium). In some embodiments, the polymer further comprises polyvinylpyridine (PVP) or polyethylenimine (PEI). In some embodiments, the polymer further comprises an optionally substituted C₄-C₂₂ alkyl group. In some embodiments, the polymer further comprises an optionally substituted C₃-C₂₂ alkyne In some embodiments, the polymer further comprises an optionally substituted C₃-C₂₂ terminal alkyne. In some embodiments, the polymer is fully quaternized. In some embodiments, the polymer is partially quaternized. In some embodiment, the ratio of quaternized amines to non-quaternized amines is about 30% to about 50%. In some embodiments, the N⁺/N ratio is about 30% to about 50%.

In one embodiment, the moiety of formula (I) is a moiety of formula (Ia):

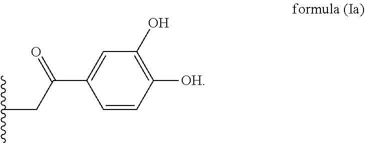 [SMILES: *CC(=O)c1ccc(O)c(O)c1]

In another aspect, the disclosure describes a polymer comprising at least one moiety of formula (II):

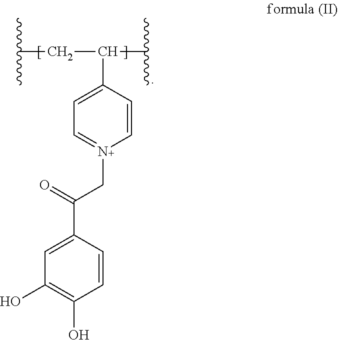

In some embodiments, the polymer further comprises at least one moiety of formula (III):

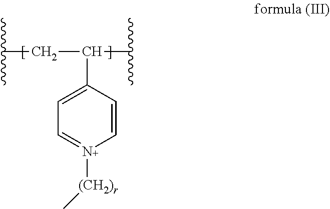

wherein r is an integer from 3 to 20. In some embodiments, r is an integer from 3 to 11. In some embodiments, r is 3. In some embodiments, r is 9. In some embodiments, the polymer consists of moieties of formula (II) and moieties of formula (III). In some embodiments, the polymer comprises moieties of formula (II) and moieties of formula (III).

In some embodiments, the polymer further comprises at least one fragment of formula (IV):

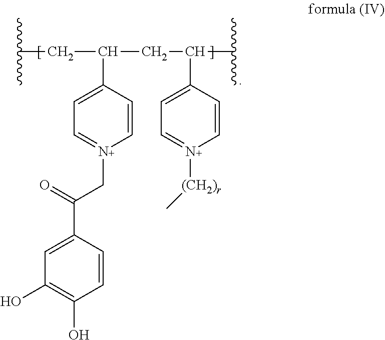

wherein in formula (IV):
r is an integer from 3 to 11. In some embodiments, r is 3. In some embodiments, r is 9.

In another aspect, the disclosure describes a polymer comprising at least one moiety of formula (V):

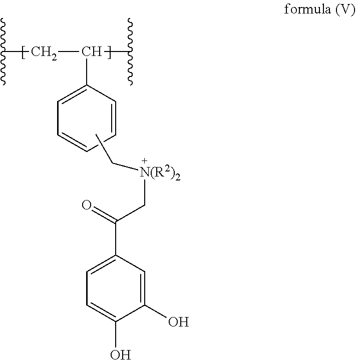

wherein in formula (V):
each R² is independently optionally substituted alkyl. In some embodiments, each R² is independently a C₁-C₄ alkyl. In some embodiments, each R² is methyl. In some embodiments, the polymer comprises a mixture of meta and para substituents of formula (V).

In some embodiments, the moiety of formula (V) is a moiety of formula (VI):

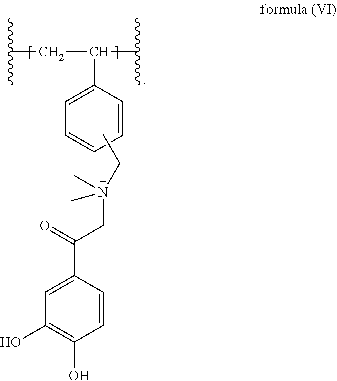

In some embodiments, the polymer comprises a mixture of meta and para substituents of formula (VI).

In some embodiments, the polymer further comprises a moiety of formula (VII):

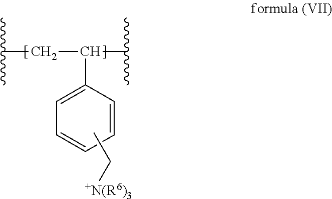

wherein in formula (VII):
each R⁶ is independently optionally substituted alkyl. In some embodiments, each R⁶ is independently a C₄-C₁₂ alkyl. In some embodiments, two R⁶ are methyl and one R⁶ is decyl. In some embodiments, the polymer comprises a mixture of meta and para substituents of formula (VII).

In some embodiments, the polymer consists of moieties of formula (V) and moieties of formula (VII). In some embodiments, the polymer comprises moieties of formula (V) and moieties of formula (VII). In some embodiments, the polymer consists of moieties of formula (VI) and moieties of formula (VII). In some embodiments, the polymer comprises moieties of formula (VI) and moieties of formula (VII).

In some embodiments, the polymer further comprises at least one moiety of formula (VIII):

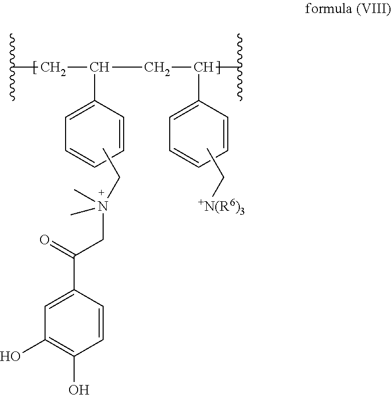

wherein in formula (VIII):
each R⁶ is independently optionally substituted alkyl.

In some embodiments, each R⁶ is independently a C₄-C₁₂ alkyl. In some embodiments, two R⁶ are methyl and one R⁶ is decyl.

In another aspect, the disclosure describes a polyethylenimine (PEI) polymer comprising at least one moiety of formula (IXa), formula (IXb), or formula (IXh):

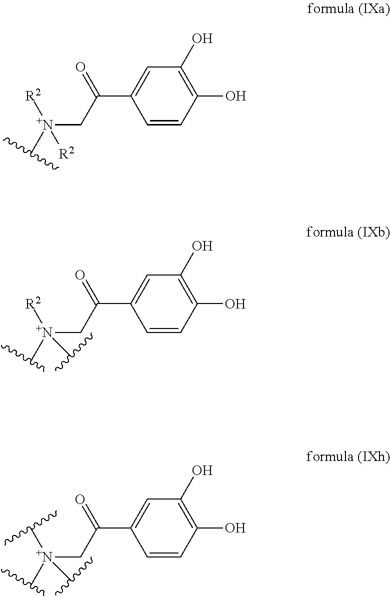

wherein in formula (IXa) and formula (IXb):
each R² is independently optionally substituted alkyl.

In some embodiments, each R² is independently methyl.

In some embodiments, the moiety of formula (IXa) is a moiety of formula (IXc):

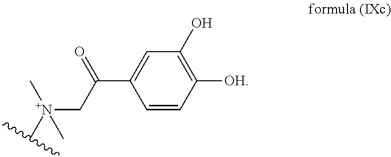 [SMILES: *[N+](C)(C)CC(=O)c1ccc(O)c(O)c1]

In some embodiments, the moiety of formula (IXb) is a moiety of formula (IXd):

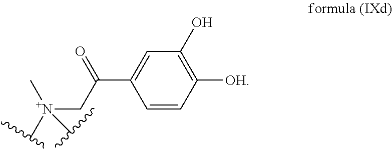 [SMILES: *[N+](*)(C)CC(=O)c1ccc(O)c(O)c1]

In some embodiments, the PEI polymer comprises moieties of formula (IXc) and moieties of formula (IXd). In some embodiments, the PEI polymer comprises moieties of formula (IXc), moieties of formula (IXd), and moieties of formula (IXh). In some embodiments, the polymer is branched, hyperbranched or linear. In some embodiments, the PEI polymer is fully alkylated. In some embodiments, the PEI polymer is fully methylated. An example of a fully methylated monomer is illustrated in FIG. 8. As would be understood by one of ordinary skill in the art, in a non-limiting example a PEI polymer comprises primary and secondary nitrogen atoms, and when fully methylated, all primary and secondary nitrogens are converted to tertiary nitrogens comprising two and one methyl groups, respectively. In a non-limiting embodiment, the tertiary nitrogens can be quaternized following treatment with catechol compounds described herein (such as Compounds 1001-1003), resulting in mixture of moieties of formula (IXc) and/or moieties of formula (IXd) and/or moieties of formula (IXh). In a non-limiting example, remaining tertiary nitrogens can be converted into quaternary nitrogens comprising an optionally substituted C₄-C₂₂ alkyl group, as would be understood by one of ordinary skill in the art. In some embodiments, the PEI polymer further comprises at least one optionally substituted C₄-C₂₂ alkyl group. In some embodiments, the PEI polymer is partially quaternized. In some embodiments, the PEI polymer is fully quaternized. In some embodiments, the molar ratio of the total moieties of formula (IXa) and moieties of formula (IXb) to the optionally substituted C₄-C₂₂ alkyl group is about 0.05≤x≤0.5:(1-x). In some embodiments, the PEI polymer is fully quaternized. In some embodiments, the molar ratio of the total moieties of formula (IXa) and moieties of formula (IXb) and moieties of formula (IXh) to the optionally substituted C₄-C₂₂ alkyl group is about 0.05≤x≤0.5:(1-x). In some embodiments, the PEI polymer consists of fully methylated monomers, wherein each monomer is fully quaternized and consists of moieties of formula (IXa), moieties of formula (IXb), and optionally substituted C₄-C₂₂ alkyl groups. In some embodiments, the PEI polymer consists of fully methylated monomers, wherein each monomer is fully quaternized and consists of moieties of formula (IXa), moieties of formula (IXb), moieties of formula (IXh), and optionally substituted C₄-C₂₂ alkyl groups. In some embodiments, the molar ratio of the total moieties of formula (IXa) and moieties of formula (IXb) to the optionally substituted C₄-C₂₂ alkyl groups is about 0.05≤x≤0.5:(1-x). In some embodiments, the molar ratio of the total moieties of formula (IXa) and moieties of formula (IXb) to the optionally substituted C₄-C₂₂ alkyl groups is about 0.1:0.9. In some embodiments, the molar ratio of the total moieties of formula (IXa), moieties of formula (IXb), and moieties of formula (IXh) to the optionally substituted C₄-C₂₂ alkyl groups is about 0.05≤x≤0.5:(1-x). In some embodiments, the molar ratio of the total moieties of formula (IXa), moieties of formula (IXb), and moieties of formula (IXh) to the optionally substituted C₄-C₂₂ alkyl groups is about 0.1:0.9. In some embodiments, the PEI polymer consists of fully methylated monomers, wherein each monomer is fully quaternized and consists of moieties of formula (IXc), moieties of formula (IXd), and optionally substituted C₄-C₂₂ alkyl groups. In some embodiments, the molar ratio of the total moieties of formula (IXc) and moieties of formula (IXd) to the optionally substituted C₄-C₂₂ alkyl groups is about 0.05≤x≤0.5:(1-x). In some embodiments, the molar ratio of the total moieties of formula (IXc) and moieties of formula (IXd) to the optionally substituted C₄-C₂₂ alkyl groups is about 0.1:0.9. In some embodiments, the PEI polymer consists of fully methylated monomers, wherein each monomer is fully quaternized and consists of moieties of formula (IXc), moieties of formula (IXd), moieties of formula (IXh), and optionally substituted C₄-C₂₂ alkyl groups. In some embodiments, the molar ratio of the total moieties of formula (IXc), moieties of formula (IXd), and moieties of formula (IXh) to the optionally substituted C₄-C₂₂ alkyl groups is about 0.05≤x≤0.5:(1-x). In some embodiments, the molar ratio of the total moieties of formula (IXc), moieties of formula (IXd), and moieties of formula (IXh) to the optionally substituted C₄-C₂₂ alkyl groups is about 0.1:0.9.

In another aspect, the disclosure describes a PEI polymer comprising at least one moiety of formula (IXe), or substructures thereof:

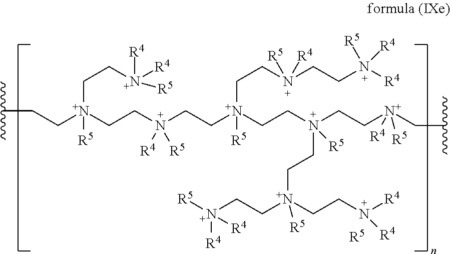

wherein in formula (IXe):
each R⁴ is independently optionally substituted alkyl; and
each R⁵ is independently optionally substituted alkyl or a moiety of formula (Ia):

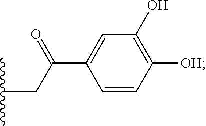 [SMILES: *CC(=O)c1ccc(O)c(O)c1]

with the proviso that at least one R⁵ is a moiety of formula (Ia):

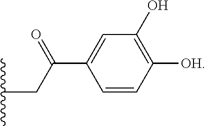 [SMILES: *CC(=O)c1ccc(O)c(O)c1]

In some embodiments, each R⁴ is independently C₁-C₃ alkyl. In some embodiments, each R⁴ is methyl. In some embodiments, 1, 2, 3, 4, 5, 6, 7, 8, 9, 10, or 11 R⁵ are moieties of formula (Ia):

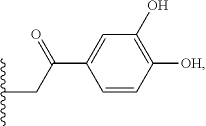 [SMILES: *CC(=O)c1ccc(O)c(O)c1]

and the remaining R⁵ are C₅-C₁₀ alkyl. In some embodiments, 1, 2, 3, 4, 5, 6, 7, 8, 9, 10, or 11 R⁵ are moieties of formula (Ia):

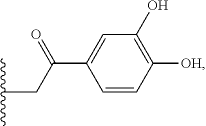 [SMILES: *CC(=O)c1ccc(O)c(O)c1]

and the remaining R⁵ are C₁₀ alkyl. In some embodiments, 1, 2, 3, 4, 5, 6, 7, 8, 9, 10, or 11 R⁵ are moieties of formula (Ia):

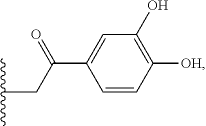 [SMILES: *CC(=O)c1ccc(O)c(O)c1]

and the remaining R⁵ are C₆ alkyl. In some embodiments, 1, 2, 3, 4, 5, 6, 7, 8, 9, 10, or 11 R⁵ are moieties of formula (Ia):

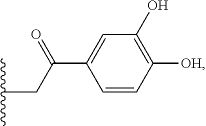 [SMILES: *CC(=O)c1ccc(O)c(O)c1]

and the remaining R⁵ are C₁₂ alkyl.

Non-limiting examples of substructures of formula (IXe) include:

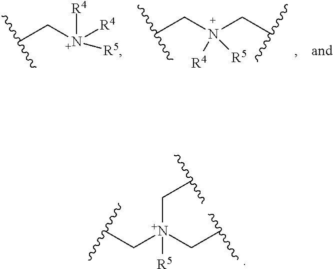

In some embodiments, in formula (IXe), the molar ratio of the number of R⁵ moieties of formula (Ia) to the number of R⁵ optionally substituted alkyl groups is about 0.05≤x≤0.5:(1-x). In some embodiments, in formula (IXe), the molar ratio of the number of R⁵ moieties of formula (Ia) to the number of R⁵ optionally substituted alkyl groups is about 0.08≤x≤0.12:(1-x). In some embodiments, in formula (IXe), the PEI polymer is fully quaternized. In some embodiments, in formula (IXe), the molar ratio of number of R⁵ moieties of formula (Ia) to the number of R⁵ optionally substituted alkyl groups are is about 0.1:0.9. In some embodiments, in formula (IXe), the alkyl group is a C₁₀ group. In some embodiments, in formula (IXe), the alkyl group is a C₆ alkyl group.

In another aspect, the disclosure describes a PEI polymer comprising at least one moiety of formula (IXe1), or substructures thereof:

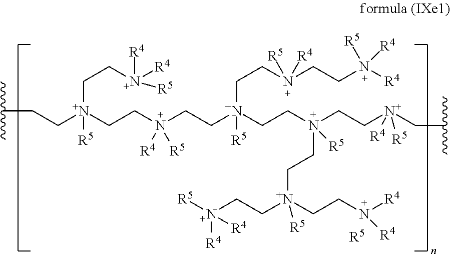

wherein in formula (IXe1):
each R⁴ is independently optionally substituted alkyl, or is absent; and
each R⁵ is independently optionally substituted alkyl, a moiety of formula (Ia)

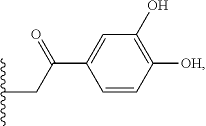 [SMILES: *CC(=O)c1ccc(O)c(O)c1]

or absent;
   with the proviso that at least one R⁵ is a moiety of formula (Ia):

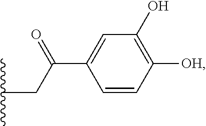 [SMILES: *CC(=O)c1ccc(O)c(O)c1]

and each nitrogen atom is trivalent or a quaternary nitrogen.

Non-limiting examples of substructures of formula (IXe1) include:

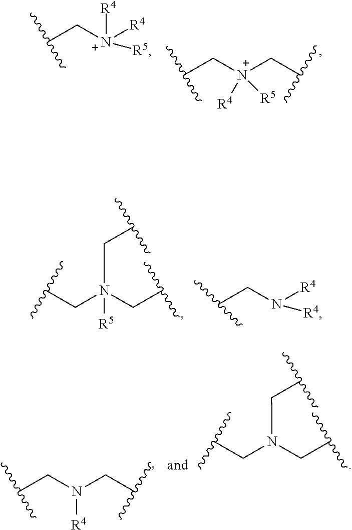

In some embodiments, each R⁴ is independently C₁-C₃ alkyl. In some embodiments, each R⁴ is methyl. In some embodiments, 1, 2, 3, 4, 5, 6, 7, 8, 9, 10, or 11 R⁵ are moieties of formula (Ia):

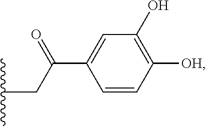 [SMILES: *CC(=O)c1ccc(O)c(O)c1]

and the remaining R⁵ are C₅-C₁₀ alkyl. In some embodiments, 1, 2, 3, 4, 5, 6, 7, 8, 9, 10, or 11 R⁵ are moieties of formula (Ia):

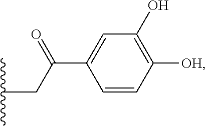 [SMILES: *CC(=O)c1ccc(O)c(O)c1]

and the remaining R⁵ are C₁₀ alkyl. In some embodiments, 1, 2, 3, 4, 5, 6, 7, 8, 9, 10, or 11 R⁵ are moieties of formula (Ia):

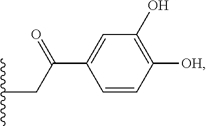 [SMILES: *CC(=O)c1ccc(O)c(O)c1]

and the remaining R⁵ are C₆ alkyl. In some embodiments, 1, 2, 3, 4, 5, 6, 7, 8, 9, 10, or 11 R⁵ are moieties of formula (Ia):

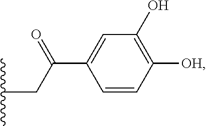 [SMILES: *CC(=O)c1ccc(O)c(O)c1]

and the remaining R⁵ are C₁₂ alkyl.

In another aspect, the disclosure describes a PEI polymer comprising at least one moiety of formula (IXf), or a substructure thereof:

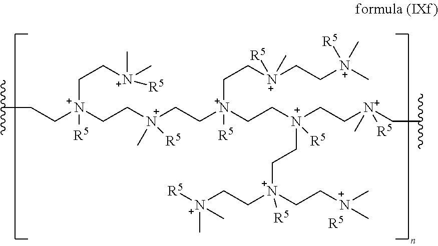

wherein in formula (IXf):
each R⁵ is independently C₁₀ alkyl or

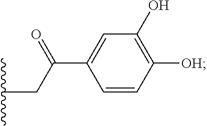 [SMILES: *CC(=O)c1ccc(O)c(O)c1]

with the proviso that at least one R⁵ is a moiety of formula (Ia):

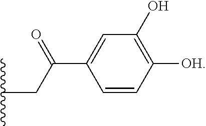 [SMILES: *CC(=O)c1ccc(O)c(O)c1]

In some embodiments, 1, 2, 3, 4, 5, 6, 7, 8, 9, 10, or 11 R⁵ are moieties of formula (Ia):

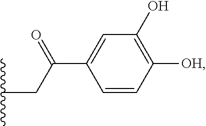 [SMILES: *CC(=O)c1ccc(O)c(O)c1]

and the remaining R⁵ are C₁₀ alkyl. In some embodiments, in formula (IXf), the molar ratio of the number of R⁵ moieties of formula (Ia) to the number of R⁵C₁₀ group groups is about 0.05≤x≤0.5:(1-x). In some embodiments, in formula (IXf), the molar ratio of the number of R⁵ moieties of formula (Ia) to the number of R⁵C₁₀ alkyl groups is about 0.06≤x≤0.12:(1-x). In some embodiments, in formula (IXf), the PEI polymer is fully quaternized. In some embodiments, in formula (IXf), the molar ratio of number of R⁵ moieties of formula (Ia) to the number of R⁵C₁₀alkyl groups are is about 0.1:0.9.

Non-limiting examples of substructures of formula (IXf) include:

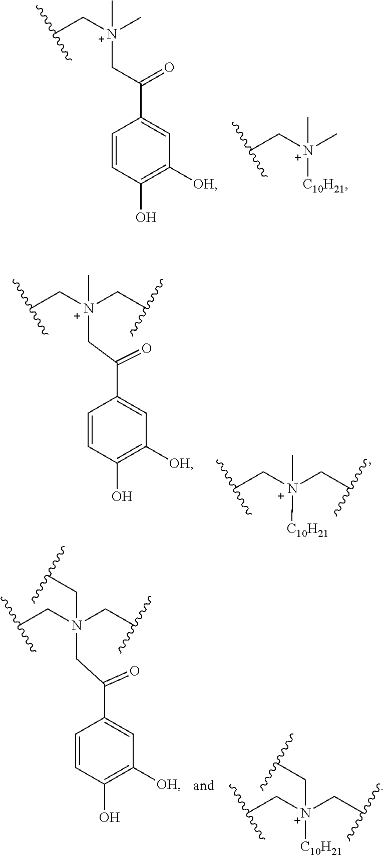

In another aspect, the disclosure describes a PEI polymer comprising at least one moiety of formula (IXg):

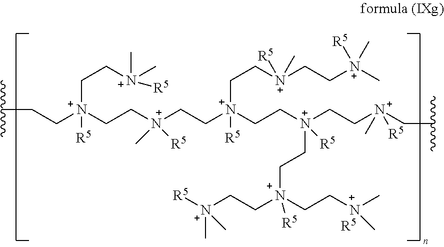

wherein in formula (IXg):
each R⁵ is independently C₆ alkyl or

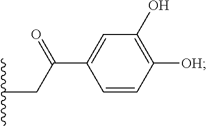 [SMILES: *CC(=O)c1ccc(O)c(O)c1]

with the proviso that at least one R⁵ is a moiety of formula (Ia):

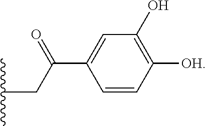 [SMILES: *CC(=O)c1ccc(O)c(O)c1]

In some embodiments, 1, 2, 3, 4, 5, 6, 7, 8, 9, 10, or 11 R⁵ are moieties of formula (Ia):

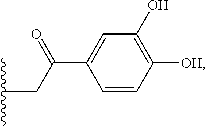 [SMILES: *CC(=O)c1ccc(O)c(O)c1]

and the remaining R⁵ are C₆ alkyl. In some embodiments, in formula (IXg), the molar ratio of the number of R⁵ moieties of formula (Ia) to the number of R⁵C₆ alkyl groups is about 0.05≤x≤0.5:(1-x). In some embodiments, in formula (IXg), the molar ratio of the number of R⁵ moieties of formula (Ia) to the number of R⁵C₆ alkyl groups is about 0.06≤x≤0.12:(1-x). In some embodiments, in formula (IXg), the PEI polymer is fully quaternized. In some embodiments, in formula (IXg), the molar ratio of number of R⁵ moieties of formula (Ia) to the number of R⁵C₆ alkyl groups is about 0.1:0.9.

Non-limiting examples of substructures of formula (IXg) include:

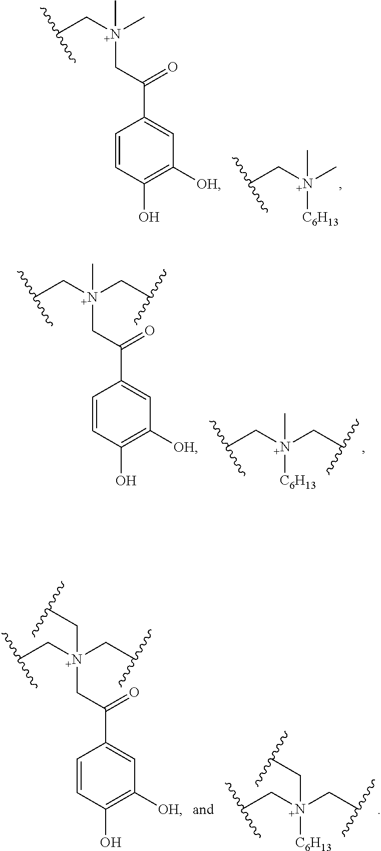

In another aspect, the disclosure describes a polymer comprising at least one moiety of formula (XI):

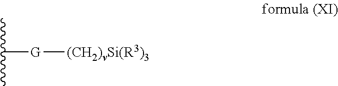

wherein in formula (XI):
G is a single bond or linking group;
each R³ is independently optionally substituted alkoxy; and
v is an integer from 3 to 10.

In some embodiments, G is a single bond. In some embodiments, G is a linking group. The linking group may be any organic moiety, as would be understood by one of ordinary skill in the art. In some embodiments, the linking group comprises optionally substituted amino, optionally substituted heterocyclyl, or carboxylate. In some embodiments, the linking group comprises a primary amino group, a secondary amino group, or a tertiary amino group. In some embodiments, the organic linker comprises dimethylamino, diethylamino, —C(O)O—, diethylcarboxylate, acetyl, optionally substituted triazole group, or optionally substituted tetrazole group. In some embodiments, the linking group comprises

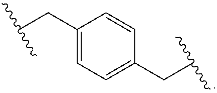 [SMILES: *Cc1ccc(C*)cc1]

In some embodiments, the linking group comprises

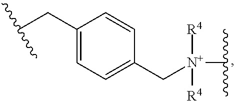

wherein each R⁴ is independently optionally substituted alkyl. In some embodiments, each 1 is independently selected from C₁-C₄ alkyl and

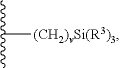

wherein each R³ is independently optionally substituted alkoxy, and v is an integer from 3 to 10. In some embodiments, each R⁴ is independently selected from methyl and

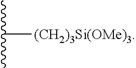

In some embodiments, one R⁴ is methyl and one R⁴ is

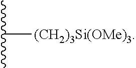

In some embodiments, the polymer is a random polymer. In some embodiments, the polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide) and poly(diallyldimethylammonium). In some embodiments, the polymer further comprises polyvinylpyridine (PVP) or polyethylenimine (PEI). In some embodiments, the polymer further comprises an optionally substituted C₄-C₂₂ alkyl group. In some embodiments, the polymer further comprises an optionally substituted C₃-C₂₂ alkyne. In one embodiment, the optionally substituted C₃-C₂₂ alkyne is a terminal alkyne. In some embodiments, the polymer is partially quaternized. In some embodiments, the polymer is fully quaternized.

In some embodiments, v is 3.

In some embodiments, R³ is methoxy.

In another aspect, the disclosure describes polymer comprising at least one moiety of formula (XII) and at least one moiety of formula (XIII):

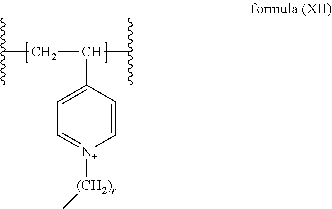

wherein in formula (XII):
r is an integer from 3 to 11;

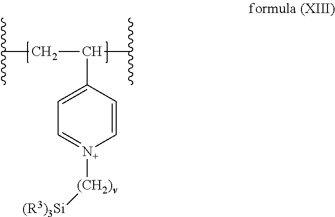

wherein in formula (XIII):
each R³ is independently optionally substituted alkoxy; and
v is an integer from 3 to 10;
with the proviso that when in formula (XIII) v is 3 and each R³ is methoxy, then in formula (XII) r is not 3.

In some embodiments, r is an integer from 4 to 11. In some embodiments, r is 9. In some embodiments, v is 3. In some embodiments, each R³ is methoxy. In some embodiments, the polymer consists of moieties of formula (XII) and moieties of formula (XII). In some embodiments, the polymer is partially quaternized. In some embodiments, the polymer is fully quaternized. In some embodiments,

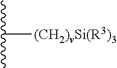

is

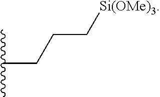

In some embodiments, the polymer comprises at least one moiety of formula (XIV):

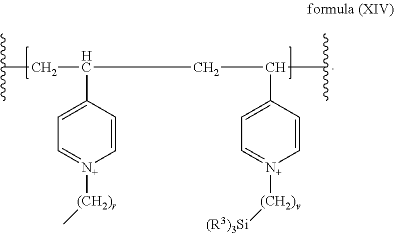

In some embodiments, when v is 3 and each R³ is methoxy, then r is not 3.

In some embodiments, r is an integer from 3 to 11. In some embodiments, r is an integer from 4 to 11. In some embodiments, r is 9. In some embodiments, v is 3. In some embodiments, each R³ is methoxy. In some embodiments,

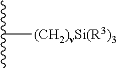

is

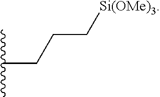

In another aspect, the disclosure describes a polyethylenimine (PEI) polymer comprising at least one moiety of formula (XIa):

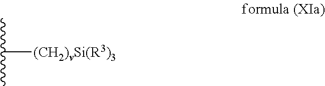

wherein in formula (XIa):
each R³ is independently optionally substituted alkoxy; and
v is an integer from 3 to 10.

In some embodiments, v is 3. In some embodiments, each R³ is methoxy. In some embodiments,

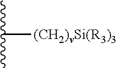

is

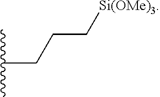

In another aspect, the disclosure describes a polyethylenimine (PEI) polymer comprising at least one of the following moieties of formula (XIa):

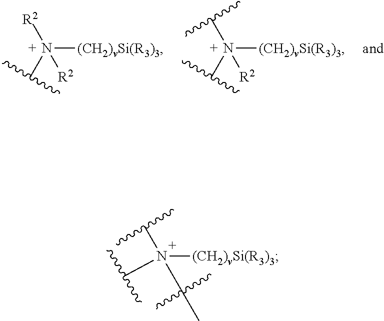

wherein each R² is independently optionally substituted alkyl. In some embodiments, R² is methyl.

In another aspect, the moiety of formula (XIa) is a moiety of formula (XIb):

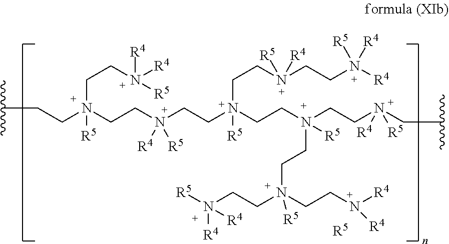

wherein in formula (XIb):
each R⁴ is independently optionally substituted alkyl; and
each R⁵ is independently optionally substituted alkyl or a moiety of formula (XIa):

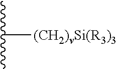

each R³ is independently optionally substituted alkoxy; and
v is an integer from 3 to 10,
   with the proviso that at least one R⁵ is a moiety of formula (XIa):

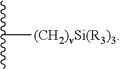

In some embodiments, v is 3. In some embodiments, each R³ is methoxy. In some embodiments, each R⁴ is independently C₁-C₃ alkyl. In some embodiments, each R⁴ is methyl. In some embodiments, each moiety of formula (XIa)

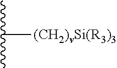

is

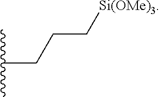

In some embodiments, 1, 2, 3, 4, 5, 6, 7, 8, 9, 10, or 11 R⁵ are moieties of formula (XIa):

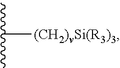

and the remaining R⁵ are C₅-C₁₀ alkyl. In some embodiments, 1, 2, 3, 4, 5, 6, 7, 8, 9, 10, or 11 R⁵ are moieties of formula (XIa):

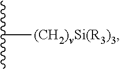

and the remaining R⁵ are C₆ alkyl. In some embodiments, 1, 2, 3, 4, 5, 6, 7, 8, 9, 10, or 11 R⁵ are

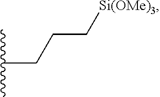

and the remaining R⁵ are C₆ alkyl.

Non-limiting examples of substructures of formula (IXb) include:

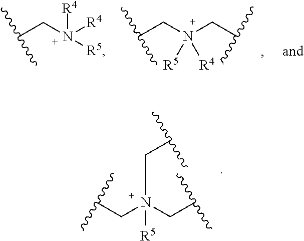

In some embodiments, in formula (XIb), the molar ratio of the number of R⁵ moieties of formula (XIa) to the number of R⁵ optionally substituted alkyl groups is about 0.05≤x≤0.5:(1-x). In some embodiments, in formula (XIb), the molar ratio of the number of R⁵ moieties of formula (XIa) to the number of R⁵ optionally substituted alkyl groups is about 0.06≤x≤0.12:(1-x). In some embodiments, in formula (XIb), the PEI polymer is fully quaternized. In some embodiments, in formula (XIb), the molar ratio of number of R⁵ moieties of formula (XIa) to the number of R⁵ optionally substituted alkyl groups are is about 0.1:0.9. In some embodiments, in formula (XIb), the alkyl group is C₆ alkyl.

In another aspect, the disclosure describes a PEI polymer comprising at least one moiety of formula (IXb1), or substructures thereof:

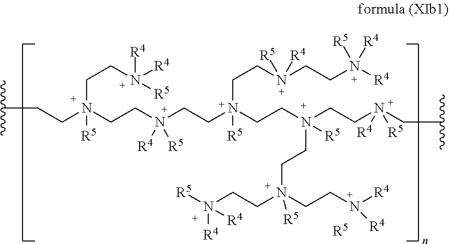

wherein in formula (IXb1):
each R⁴ is independently optionally substituted alkyl, or absent; and
each R⁵ is independently optionally substituted alkyl, a moiety of formula (XIa):

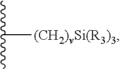

or absent;
each R³ is independently optionally substituted alkoxy; and
v is an integer from 3 to 10;
   with the proviso that at least one R⁵ is a moiety of formula (XIa):

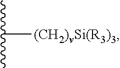

and each nitrogen atom is trivalent or a quaternary nitrogen.

Non-limiting examples of substructures of formula (IXb1) include:

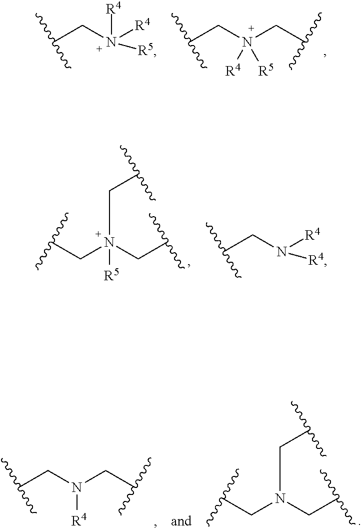

In another aspect, the moiety of formula (XIa) is a moiety of formula (XIc):

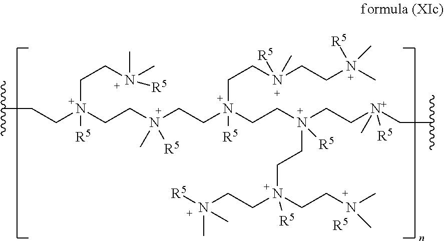

wherein in formula (XIc):
each R⁵ is independently C₆ alkyl or

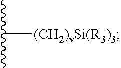

each R³ is independently optionally substituted alkoxy; and
v is an integer from 3 to 10,
   with the proviso that at least one R⁵ is

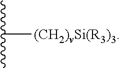

In some embodiments, v is 3. In some embodiments, each R³ is methoxy. In some embodiments,

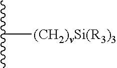

is

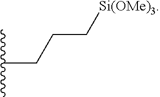

In some embodiments, 1, 2, 3, 4, 5, 6, 7, 8, 9, 10, or 11 R⁵ are

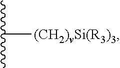

and the remaining R⁵ are C₅-C₁₀ alkyl. In some embodiments, 1, 2, 3, 4, 5, 6, 7, 8, 9, 10, or 11 R⁵ are

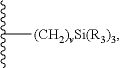

and the remaining R⁵ are C₆ alkyl. In some embodiments, 1, 2, 3, 4, 5, 6, 7, 8, 9, 10, or 11 R⁵ are

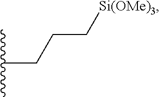

and the remaining R⁵ are C₆ alkyl.

Non-limiting examples of substructures of formula (IXf) include:

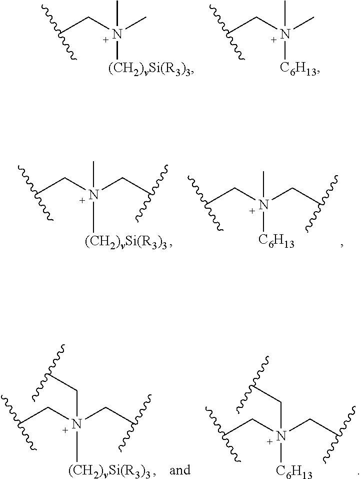

In some embodiments, in formula (XIc), the molar ratio of the number of R⁵ moieties of formula (XIa) to the number of R⁵C₆ alkyl groups is about 0.05≤x≤0.5:(1-x). In some embodiments, in formula (XIc), the molar ratio of the number of R⁵ moieties of formula (XIa) to the number of R⁵C₆ alkyl groups is about 0.06≤x≤0.12:(1-x). In some embodiments, in formula (XIc), the PEI polymer is fully quaternized. In some embodiments, in formula (XIc), the molar ratio of the number of R⁵ moieties of formula (XIa) to the number of R⁵C₆ alkyl groups is about 0.1:0.9.

In another aspect, the disclosure describes a polymer comprising at least one moiety of formula (XVIa):

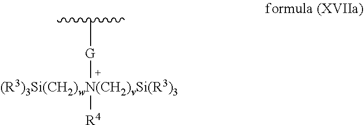

wherein in formula (XVIIa):
G is a single bond or a linking group;
each R³ is independently optionally substituted alkoxy;
R⁴ is independently optionally substituted alkyl;
v is an integer from 3 to 10; and
w is an integer from 3 to 10.

In another aspect, the disclosure describes a polymer comprising at least one moiety of formula (XVIIIa):

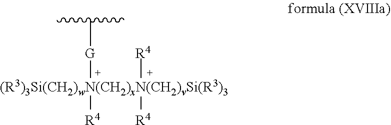

wherein in formula (XVIIIa):
G is a single bond or a linking group;
each R³ is independently optionally substituted alkoxy;
each R⁴ is independently optionally substituted alkyl;
v is an integer from 3 to 10;
w is an integer from 3 to 10; and
x is an integer from 1 to 4.

In some embodiments, G is a single bond. In some embodiments, G is a linking group. The linking group may be any organic moiety, as would be understood by one of ordinary skill in the art. In some embodiments, the linking group comprises.

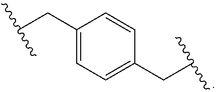 [SMILES: *Cc1ccc(C*)cc1]

In some embodiments, the linking group comprises

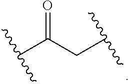 [SMILES: *CC(*)=O]

In some embodiments, w is 3. In some embodiments, v is 3. In some embodiments, x is 2. In some embodiments, each R³ is independently optionally substituted methoxy. In some embodiments, each R³ is methoxy. In some embodiments, each R⁴ is independently C₁-C₂₂ alkyl or C₁-C₂₂ haloalkyl. In some embodiments, two R⁴ are methyl and two R⁴ is independently C₁-C₂₂ alkyl or C₁-C₂₂ haloalkyl. In some embodiments, each R⁴ is independently C₁₈ alkyl or C₁₈ haloalkyl. In some embodiments, each R⁴ is independently C₁₆ alkyl or C₁₆ haloalkyl. In some embodiments, each R⁴ is independently selected from —CH₂(CH₂)ₙCH₃ and —CH₂CH₂(CF₂)ₙCF₃, wherein n is an integer from 15 to 20. In some embodiments, each R⁴ is independently selected from —CH₂(CH₂)ₙCH₃ wherein n is an integer from 14 to 20. In some embodiments, each R⁴ is independently selected from —CH₂(CH₂)ₙCH₃ wherein n is 14, 16, 18, or 20. In some embodiments, n is 14. In some embodiments, n is 16. In some embodiments, each R⁴ is independently selected from —CH₂CH₂(CF₂)ₙCF₃ wherein n is an integer from 15 to 19. In some embodiments, each R⁴ is independently selected from —CH₂CH₂(CF₂)ₙCF₃ wherein n is 15, 17, or 19. In some embodiments, n is 15. In some embodiments, n is 17.

In another aspect, the disclosure describes a polymer comprising at least one moiety of formula (XVIIh):

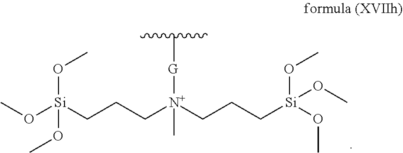

In another aspect, the disclosure describes a polymer comprising at least one moiety of formula (XVIIb):

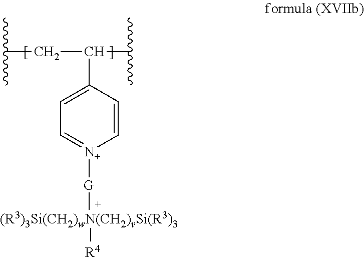

wherein in formula (XVIIb):
G is a single bond or a linking group;
each R³ is independently optionally substituted alkoxy;
R⁴ is independently optionally substituted alkyl;
v is an integer from 3 to 10; and
w is an integer from 3 to 10. In some embodiments, the linking group comprises

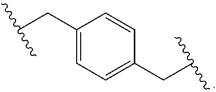 [SMILES: *Cc1ccc(C*)cc1]

In some embodiments, the linking group comprises

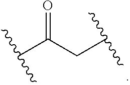 [SMILES: *CC(*)=O]

In some embodiments, each R³ is independently optionally substituted methoxy. In some embodiments, each R³ is methoxy. In some embodiments, R⁴ is independently C₁-C₂₂ alkyl or C₁-C₂₂ haloalkyl. In some embodiments, R⁴ is independently C₁₈ alkyl or C₁₈ haloalkyl. In some embodiments, R⁴ is independently C₁₆ alkyl or C₁₆ haloalkyl. In some embodiments, R⁴ is C₁-C₂₂ alkyl or C₁-C₂₂ haloalkyl. In some embodiments, R⁴ is methyl. In some embodiments, R⁴ is selected from —CH₂(CH₂)ₙCH₃ and —CH₂CH₂(CF₂)ₙCF₃, wherein n is an integer from 15 to 20. In some embodiments, R⁴ is —CH₂(CH₂)ₙCH₃ wherein n is an integer from 14 to 20. In some embodiments, R⁴ is —CH₂(CH₂)ₙCH₃ wherein n is 14, 16, 18, or 20. In some embodiments, n is 14. In some embodiments, n is 16. In some embodiments, R⁴ is —CH₂CH₂(CF₂)ₙCF₃ wherein n is an integer from 15 to 19. In some embodiments, R⁴ is —CH₂CH₂(CF₂)ₙCF₃ wherein n is 15, 17, or 19. In some embodiments, n is 15. In some embodiments, n is 17. In some embodiments, w is 3. In some embodiments, v is 3.

In another aspect, the disclosure describes a polymer comprising at least one moiety of formula (XVIIj):

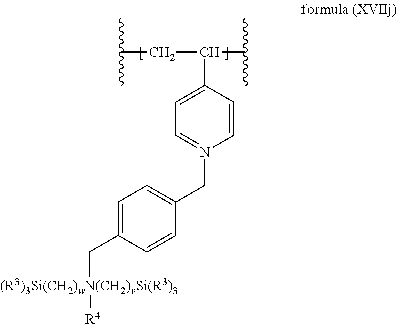

wherein in formula (XVIIj):
each R³ is independently optionally substituted alkoxy;
R⁴ is independently optionally substituted alkyl;
v is an integer from 3 to 10; and
w is an integer from 3 to 10. In some embodiments, each R³ is independently optionally substituted methoxy. In some embodiments, each R³ is methoxy. In some embodiments, R⁴ is independently C₁-C₂₂ alkyl or C₁-C₂₂ haloalkyl. In some embodiments, R⁴ is C₁-C₂₂ alkyl or C₁-C₂₂ haloalkyl. In some embodiments, each R⁴ is independently C₁₈ alkyl or C₁₈ haloalkyl. In some embodiments, each R⁴ is independently C₁₆ alkyl or C₁₆ haloalkyl. In some embodiments, R⁴ is methyl. In some embodiments, R⁴ is selected from —CH₂(CH₂)ₙCH₃ and —CH₂CH₂(CF₂)ₙCF₃, wherein n is an integer from 15 to 20. In some embodiments, R⁴ is —CH₂(CH₂)ₙCH₃ wherein n is an integer from 14 to 20. In some embodiments, R⁴ is —CH₂(CH₂)ₙCH₃ wherein n is 14, 16, 18, or 20. In some embodiments, n is 14. In some embodiments, n is 16. In some embodiments, R⁴ is —CH₂CH₂(CF₂)ₙCF₃ wherein n is an integer from 15 to 19. In some embodiments, R⁴ is —CH₂CH₂(CF₂)ₙCF₃ wherein n is 15, 17, or 19. In some embodiments, n is 15. In some embodiments, n is 17. In some embodiments, w is 3. In some embodiments, v is 3.

In some embodiments, the polymer further comprises at least one moiety of formula (III):

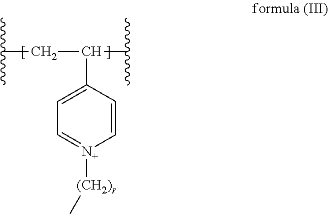

wherein r is an integer from 3 to 20. In some embodiments, r is an integer from 3 to 11. In some embodiments, r is 3. In some embodiments, the polymer consists of moieties of formula (XVIIb) and moieties of formula (III). In some embodiments, the polymer comprises moieties of formula (XVIIb) and moieties of formula (III). In some embodiments, the polymer consists of moieties of formula (XVIIj) and moieties of formula (III). In some embodiments, the polymer comprises moieties of formula (XVIIj) and moieties of formula (III).

In some embodiments, the polymer comprises at least one moiety of formula (XVIId):

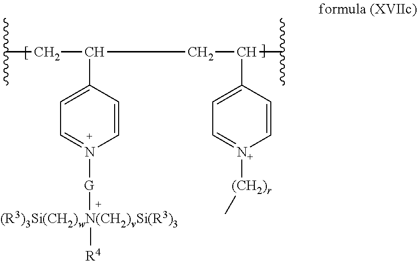

wherein in formula (XVIIc):
G is a single bond or a linking group;
each R³ is independently optionally substituted alkoxy;
R⁴ is independently optionally substituted alkyl;
r is an integer from 3 to 11;
v is an integer from 3 to 10; and
w is an integer from 3 to 10. In some embodiments, the linking group comprises

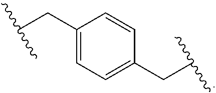 [SMILES: *Cc1ccc(C*)cc1]

In some embodiments, the linking group comprises

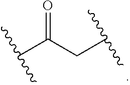 [SMILES: *CC(*)=O]

In some embodiments, each R³ is independently optionally substituted methoxy. In some embodiments, each R³ is methoxy. In some embodiments, R⁴ is independently C₁-C₂₂ alkyl or C₁-C₂₂ haloalkyl. In some embodiments, R⁴ is C₁-C₂₂ alkyl or C₁-C₂₂ haloalkyl. In some embodiments, R⁴ is independently C₁₈ alkyl or C₁₈ haloalkyl. In some embodiments, R⁴ is independently C₁₆ alkyl or C₁₆ haloalkyl. In some embodiments, R⁴ is methyl. In some embodiments, R⁴ is selected from —CH₂(CH₂)ₙCH₃ and —CH₂CH₂(CF₂)ₙCF₃, wherein n is an integer from 15 to 20. In some embodiments, R⁴ is —CH₂(CH₂)ₙCH₃ wherein n is an integer from 14 to 20. In some embodiments, R⁴ is —CH₂(CH₂)ₙCH₃ wherein n is 14, 16, 18, or 20. In some embodiments, n is 14. In some embodiments, n is 16. In some embodiments, R⁴ is —CH₂CH₂(CF₂)ₙCF₃ wherein n is an integer from 15 to 19. In some embodiments, R⁴ is —CH₂CH₂(CF₂)ₙCF₃ wherein n is 15, 17, or 19. In some embodiments, n is 15. In some embodiments, n is 17. In some embodiments, w is 3. In some embodiments, v is 3.

In some embodiments, the moiety of formula (XVIIb) is a moiety of formula (XVIId):

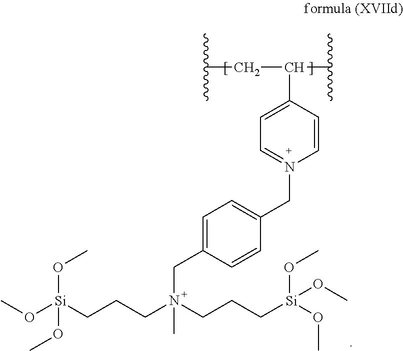

In some embodiments, the polymer consists of moieties of formula (XVIId) and moieties of formula (III). In some embodiments, the polymer comprises moieties of formula (XVIId) and moieties of formula (III).

In another aspect, the disclosure describes a polymer comprising at least one moiety of formula (XVIIe):

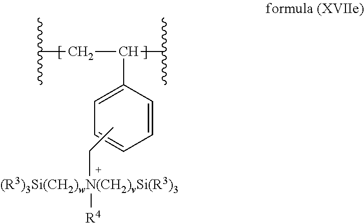

wherein in formula (XVIIe):
each R³ is independently optionally substituted alkoxy;
R⁴ is independently optionally substituted alkyl;
v is an integer from 3 to 10; and
w is an integer from 3 to 10. In some embodiments, R⁴ is independently C₁-C₂₂ alkyl or C₁-C₂₂ haloalkyl. In some embodiments, R⁴ is C₁-C₂₂ alkyl or C₁-C₂₂ haloalkyl. In some embodiments, R⁴ is independently C₁₈ alkyl or C₁₈ haloalkyl. In some embodiments, R⁴ is independently C₁₆ alkyl or C₁₆ haloalkyl. In some embodiments, R⁴ is methyl. In some embodiments, R⁴ is selected from —CH₂(CH₂)ₙCH₃ and —CH₂CH₂(CF₂)ₙCF₃, wherein n is an integer from 15 to 20. In some embodiments, R⁴ is —CH₂(CH₂)ₙCH₃ wherein n is an integer from 14 to 20. In some embodiments, R⁴ is —CH₂(CH₂)ₙCH₃ wherein n is 14, 16, 18, or 20. In some embodiments, n is 14. In some embodiments, n is 16. In some embodiments, R⁴ is —CH₂CH₂(CF₂)ₙCF₃ wherein n is an integer from 15 to 19. In some embodiments, R⁴ is —CH₂CH₂(CF₂)ₙCF₃ wherein n is 15, 17, or 19. In some embodiments, n is 15. In some embodiments, n is 17. In some embodiments, w is 3. In some embodiments, v is 3.

In some embodiments, the moiety of formula (XVIIe) is a moiety of formula (XVIIf):

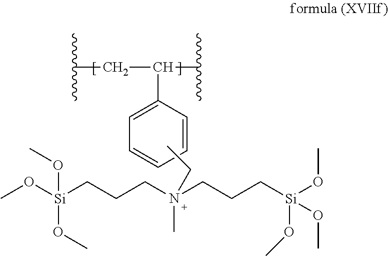

wherein in formula (XVIIf):
each R² is independently optionally substituted alkyl. In some embodiments, each R² is independently a C₁-C₄ alkyl.

In some embodiments, the polymer further comprises a moiety of formula (VII):

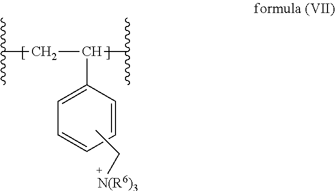

wherein in formula (VII):
each R⁶ is independently optionally substituted alkyl. In some embodiments, each R⁶ is independently a C₄-C₁₂ alkyl. In some embodiments, two R⁶ are methyl and one R⁶ is decyl.

In some embodiments, the polymer further comprises at least one moiety of formula (XVIIg):

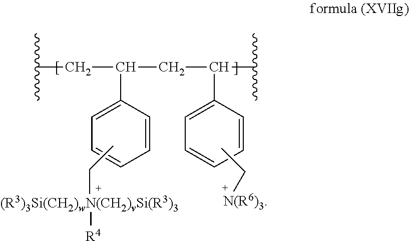

wherein in formula (XVIIg):
each R³ is independently optionally substituted alkoxy;
each R⁴ and R⁶ is independently optionally substituted alkyl;
v is an integer from 3 to 10; and
w is an integer from 3 to 10. In some embodiments, each R³ is independently a C₄-C₁₂ alkyl. In some embodiments, two R³ are methyl and one R³ is decyl. In some embodiments, w is 3. In some embodiments, v is 3.

In some embodiments, the polymer consists of moieties of formula (XVIIe) and moieties of formula (VII). In some embodiments, the polymer comprises moieties of formula (XVIIe) and moieties of formula (VII). In some embodiments, the polymer consists of moieties of formula (XVIIf) and moieties of formula (VII). In some embodiments, the polymer comprises moieties of formula (XVIIf) and moieties of formula (VII).

In another aspect, the disclosure describes a polymer comprising at least one moiety of formula (XXI):

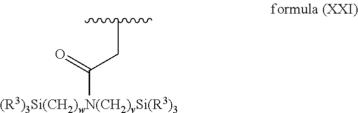

wherein in formula (XXI):
each R³ is independently optionally substituted alkoxy;
v is an integer from 3 to 10; and
w is an integer from 3 to 10. In some embodiments, each R³ is independently optionally substituted methoxy. In some embodiments, each R³ is methoxy. In some embodiments, w is 3. In some embodiments, v is 3. In some embodiments, the at least one moiety of formula (XXI) is covalently bonded to an amine moiety of the polymer. In some embodiments, the at least one moiety of formula (XXI) is covalently bonded to a quaternizable amine moiety of the polymer. In some embodiments, the at least one moiety of formula (XXI) is covalently bonded to a tertiary amine moiety of the polymer. Any polymer comprising one or more tertiary amine moieties and/or one or more quaternizable nitrogens can further comprise a moiety of formula (XXI), thereby forming a quaternary amine moiety. Non-limiting examples of suitable polymers comprising tertiary amines include polyvinylpyridine and alkylated polyethylenimine (PEI) (e.g. methylated PEI), poly(n-vinyl imidazole), polylysine, poly[2 (dimethylamino)ethyl methacrylate], Poly(vinyl benzyl amine), poly(vinyl methyl benzylamine), polyvinyldimethylbenzylamine, and hydrolyzed polyvinylpyrrolidone.

In another aspect, the disclosure describes a polymer comprising at least one moiety of formula (XXIa):

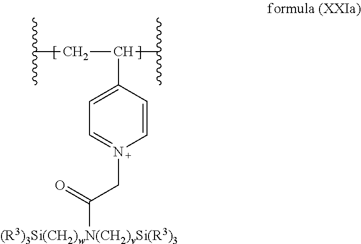

wherein in formula (XXIa):
each R³ is independently optionally substituted alkoxy;
v is an integer from 3 to 10; and
w is an integer from 3 to 10.

In some embodiments, each R³ is independently optionally substituted methoxy. In some embodiments, each R³ is methoxy. In some embodiments, w is 3. In some embodiments, v is 3.

In another aspect, the disclosure describes a polymer comprising at least one moiety of formula (XXIb):

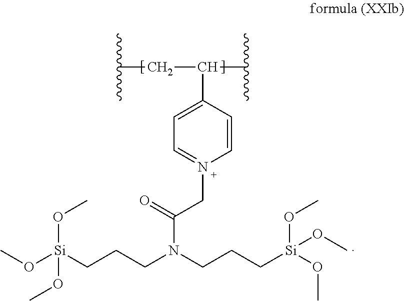

In some embodiments, the polymer further comprises at least one moiety of formula (III):

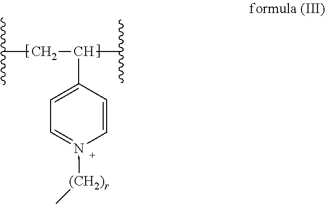

wherein r is an integer from 3 to 20. In some embodiments, r is an integer from 3 to 11. In some embodiments, r is 3. In some embodiments, the polymer consists of moieties of formula (XXIa) and moieties of formula (III). In some embodiments, the polymer comprises moieties of formula (XXIa) and moieties of formula (III). In some embodiments, the polymer consists of moieties of formula (XXIb) and moieties of formula (III). In some embodiments, the polymer comprises moieties of formula (XXIb) and moieties of formula (III).

In some embodiments, the polymer comprises at least one moiety of formula (XXId):

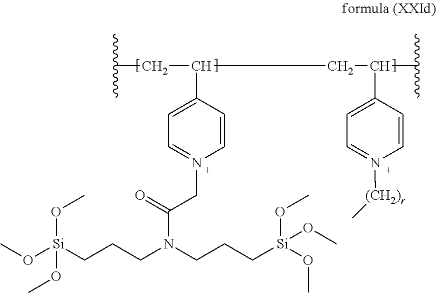

wherein in formula (XXId):
r is an integer from 3 to 11. In some embodiments, w is 3. In some embodiments, v is 3. In some embodiments, r is 3. In some embodiments, r is 9.

In another aspect, the disclosure describes a polymer comprising at least one moiety of formula (XXII):

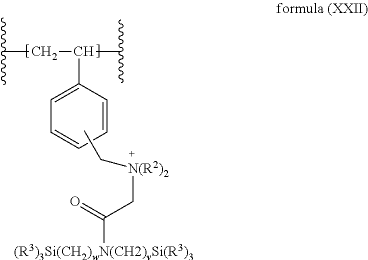

wherein in formula (XXII):
each R² is independently optionally substituted alkyl;
each R³ is independently optionally substituted alkoxy;
v is an integer from 3 to 10; and
w is an integer from 3 to 10. In some embodiments, each R² is independently a C₁-C₄ alkyl. In some embodiments, each R² is methyl. In some embodiments, the polymer comprises a mixture of meta and para substituents of formula (XXII). In some embodiments, each R³ is independently optionally substituted methoxy. In some embodiments, each R³ is methoxy. In some embodiments, w is 3. In some embodiments, v is 3.

In some embodiments, the moiety of formula (XXII) is a moiety of formula (XXIIa):

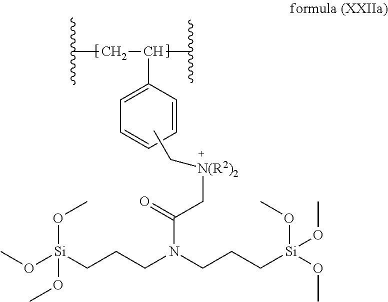

wherein in formula (XXII):
each R² is independently optionally substituted alkyl. In some embodiments, each R² is independently a C₁-C₄ alkyl. In some embodiments, each R² is methyl.

In some embodiments, the moiety of formula (XXII) is a moiety of formula (XXIIb):

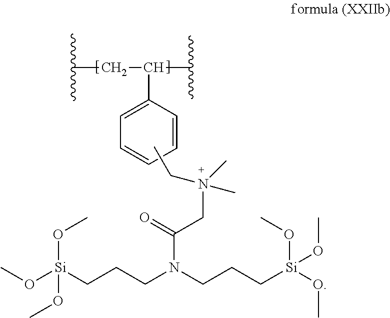

In some embodiments, the polymer further comprises a moiety of formula (VII):

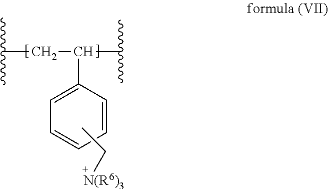

wherein in formula (VII):
each R⁶ is independently optionally substituted alkyl. In some embodiments, each R⁶ is independently a C₄-C₁₂ alkyl. In some embodiments, two R⁶ are methyl and one R³ is decyl.

In some embodiments, the polymer consists of moieties of formula (XXII) and moieties of formula (VII). In some embodiments, the polymer comprises moieties of formula (XXII) and moieties of formula (VII). In some embodiments, the polymer consists of moieties of formula (XXIIa) and moieties of formula (VII). In some embodiments, the polymer comprises moieties of formula (XXIIa) and moieties of formula (VII). In some embodiments, the polymer consists of moieties of formula (XXIIb) and moieties of formula (VII). In some embodiments, the polymer comprises moieties of formula (XXIIb) and moieties of formula (VII).

In some embodiments, the polymer further comprises at least one moiety of formula (XXIIc):

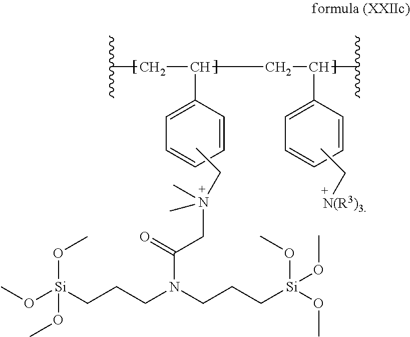

In some embodiments, each R³ is independently a C₄-C₁₂ alkyl. In some embodiments, two R³ are methyl and one R³ is decyl.

In some embodiments, the PEI polymer is branched, hyperbranched or linear. In some embodiments, the PEI polymer is fully methylated. An example of a fully methylated monomer is illustrated in FIG. 8. In some embodiments, the PEI polymer further comprises at least one optionally substituted C₄-C₂₂ alkyl group. In some embodiments, the polymer is partially quaternized. In some embodiments, the PEI polymer is fully quaternized. In some embodiments, the molecular weight of the PEI polymer has a molecular weight in a range of about 160 kda and about 750 kda. In some embodiments, the molecular weight of the PEI polymer has a molecular weight of about 160 kda, about 170 kda, about 180 kda, about 190 kda, about 200 kda, about 210 kda, about 220 kda, about 230 kda, about 240 kda, about 250 kda, about 260 kda, about 270 kda, about 280 kda, about 290 kda, about 300 kda, about 310 kda, about 320 kda, about 330 kda, about 340 kda, about 350 kda, about 360 kda, about 370 kda, about 380 kda, about 390 kda, about 400 kda, about 410 kda, about 420 kda, about 430 kda, about 440 kda, about 450 kda, about 460 kda, about 470 kda, about 480 kda, about 490 kda, about 400 kda, about 510 kda, about 520 kda, about 530 kda, about 540 kda, about 550 kda, about 560 kda, about 570 kda, about 580 kda, about 590 kda, about 600 kda, about 610 kda, about 620 kda, about 630 kda, about 640 kda, about 650 kda, about 660 kda, about 670 kda, about 680 kda, about 690 kda, about 700 kda, about 710 kda, about 720 kda, about 730 kda, about 740 kda, or about 750 kda.

In some embodiments, the molar ratio of the moieties of formula (XIa) to the optionally substituted C₄-C₂₂ alkyl group is about 0.05≤x≤0.5:(1-x). In some embodiments, the PEI polymer is fully quaternized. In some embodiments, the PEI polymer consists of fully methylated monomers, wherein each monomer is fully quaternized and consists of moieties of formula (XIa) and optionally substituted C₄-C₂₂ alkyl groups. In some embodiments, the ratio of the total moieties of formula (XIa) to the optionally substituted C₄-C₂₂ alkyl groups is about 0.05≤x≤0.5:(1-x). In some embodiments, the moieties of formula (XIa) to the optionally substituted C₄-C₂₂ alkyl groups are present in a molar ratio of about 0.1:0.9. In some embodiments, the C4-C₂₂ alkyl group is a C₁₀ group. The amount of a particular moiety or monomer, whether the relative amount or a quantitative amount, present in a polymer or copolymer as described herein, can be determined and described using methods as understood by one of ordinary skill in the art. In one embodiment, the amount of each moiety present in a polymer is described by its molar ratio. In some embodiments, the molar ratio is 0.05, 0.06, 0.07, 0.08, 0.09, 0.11, 0.11, 0.12, 0.13, 0.14, 0.15, 0.16, 0.17. 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50. In some embodiments, the molar ratio is 0.05≤x≤0.5. In some embodiments, the molar ratio is 0.05≤x≤0.2. In some embodiments, the molar ratio of formula (I) is 0.05≤x≤0.5. In some embodiments, the molar ratio of formula (II) is 0.05≤x≤0.5. In some embodiments, the molar ratio of formula (III) is 0.5≤x≤0.95. In some embodiments, the molar ratio of formula (V) is 0.05≤x≤0.5. In some embodiments, the molar ratio of formula (VI) is 0.05≤x≤0.5. In some embodiments, the molar ratio of formula (VII) is 0.5≤x≤0.95. In some embodiments, the molar ratio of formula (IX) is 0.05≤x≤0.5. In some embodiments, the molar ratio of formula (IXa) is 0.05≤x≤0.5. In some embodiments, the molar ratio of formula (XI) is 0.05≤x≤0.2. In some embodiments, the molar ratio of formula (XII) is 0.8≤x≤0.95. In some embodiments, the molar ratio of formula (XIII) is 0.05≤x≤0.2. In some embodiments, the molar ratio of formula (XIa) is 0.05≤x≤0.2. In some embodiments, the molar ratio of formula (XXIa) is 0.05≤x≤0.2. In some embodiments, the molar ratio of formula (XXIb) is 0.05≤x≤0.2. In some embodiments, the molar ratio of formula (XXII) is 0.05≤x≤0.2. In some embodiments, the molar ratio of formula (XXIIa) is 0.05≤x≤0.2. In some embodiments, the molar ratio of formula (XXIIb) is 0.05≤x≤0.2. In some embodiments, the molar ratio of formula (XVIIe) is 0.05≤x≤0.2. In some embodiments, the molar ratio of formula (XVIIf) is 0.05≤x≤0.2. In some embodiments, a polymer comprises two different moieties, and the moieties are present in a ratio of 0.05≤x≤0.5:(1-x) or 0.05≤x≤0.2:(1-x). In some embodiments, x is 0.05, 0.06, 0.07, 0.08, 0.09, 0.11, 0.11, 0.12, 0.13, 0.14, 0.15, 0.16, 0.17. 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50. In some embodiments, the moieties of formula (II) and moieties of formula (III) are present in a molar ratio of 0.05≤x≤0.5:(1-x). In one embodiment, the moieties of formula (II) and moieties of formula (III) are present in a molar ratio of 0.05≤x≤0.2:(1-x). In one embodiment, the moieties of formula (II) and moieties of formula (III) are present in a molar ratio of about 0.06:0.94. In some embodiments, the moieties of formula (V) and moieties of formula (VII) are present in a molar ratio of 0.05≤x≤0.5:(1-x). In one embodiment, the moieties of formula (V) and moieties of formula (VII) are present in a molar ratio of 0.05≤x≤0.2:(1-x). In one embodiment, the moieties of formula (V) and moieties of formula (VII) are present in a molar ratio of about 0.1:0.9. In some embodiments, the moieties of formula (VI) and moieties of formula (VII) are present in a molar ratio of 0.05≤x≤0.5:(1-x). In one embodiment, the moieties of formula (VI) and moieties of formula (VII) are present in a molar ratio of 0.05≤x≤0.2:(1-x). In one embodiment, the moieties of formula (VI) and moieties of formula (VII) are present in a molar ratio of about 0.1:0.9. In some embodiments, the moieties of formula (XII) and moieties of formula (XIII) are present in a molar ratio of 0.05≤x≤0.5:(1-x). In some embodiments, the moieties of formula (XII) and moieties of formula (XIII) are present in a molar ratio of about 0.05:0.95. In one embodiment, the moieties of formula (XII) and moieties of formula (XIII) are present in a molar ratio of 0.05≤x≤0.2:(1-x). In some embodiments, the molar ratio is 0.05:0.95. In some embodiments, the molar ratio is about 0.1:0.9. In some embodiments, the molar ratio is about 0.06:0.94. In some embodiments, the moieties of formula (XXIa) and moieties of formula (III) are present in a molar ratio of 0.05≤x≤0.5:(1-x). In some embodiments, the moieties of formula (XXIb) and moieties of formula (III) are present in a molar ratio of 0.05≤x≤0.5:(1-x). In some embodiments, the moieties of formula (XXIa) and moieties of formula (III) are present in a molar ratio of about 0.10:0.90. In some embodiments, the moieties of formula (XXIb) and moieties of formula (III) are present in a molar ratio of about 0.10:0.90. In some embodiments, the moieties of formula (XXII) and moieties of formula (VIII) are present in a molar ratio of 0.05≤x≤0.5:(1-x). In some embodiments, the moieties of formula (XXIIa) and moieties of formula (VIII) are present in a molar ratio of 0.05≤x≤0.5:(1-x). In some embodiments, the moieties of formula (XXIIb) and moieties of formula (VIII) are present in a molar ratio of 0.05≤x≤0.5:(1-x). In some embodiments, the moieties of formula (XXII) and moieties of formula (VIII) are present in a molar ratio of about 0.10:0.90. In some embodiments, the moieties of formula (XXII) and moieties of formula (VIII) are present in a molar ratio of about 0.10:0.90. In some embodiments, the moieties of formula (XXIIb) and moieties of formula (VIII) are present in a molar ratio of about 0.10:0.90. In some embodiments, the moieties of formula (XVIIb) and moieties of formula (III) are present in a molar ratio of 0.05≤x≤0.5:(1-x). In some embodiments, the moieties of formula (XVIIb) and moieties of formula (III) are present in a molar ratio of about 0.10:0.90. In some embodiments, the moieties of formula (XVIId) and moieties of formula (III) are present in a molar ratio of 0.05≤x≤0.5:(1-x). In some embodiments, the moieties of formula (XVIId) and moieties of formula (III) are present in a molar ratio of about 0.10:0.90. In some embodiments, the moieties of formula (XVIIe) and moieties of formula (VII) are present in a molar ratio of 0.05≤x≤0.5:(1-x). In some embodiments, the moieties of formula (XVIIe) and moieties of formula (VII) are present in a molar ratio of about 0.10:0.90. In some embodiments, the moieties of formula (XXIa) and moieties of formula (III) are present in a molar ratio of 0.05≤x≤0.5:(1-x). In some embodiments, the moieties of formula (XXIa) and moieties of formula (III) are present in a molar ratio of about 0.10:0.90. In some embodiments, the moieties of formula (XXIb) and moieties of formula (III) are present in a molar ratio of 0.05≤x≤0.5:(1-x). In some embodiments, the moieties of formula (XXIb) and moieties of formula (VII) are present in a molar ratio of about 0.10:0.90.

In some embodiments, each R⁵ is independently C₄-C₂₂ alkyl.

In another aspect, the disclosure describes a polymer comprising at least one moiety of formula (Ie):

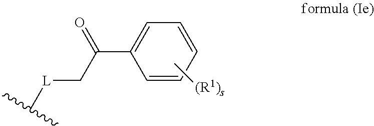

wherein in formula (Ie):
L is a single bond or an organic linker;
each R¹ is OH; and
s is an integer from 0 to 3.

In another aspect, the disclosure describes a polymer comprising at least one moiety of formula (IIb):

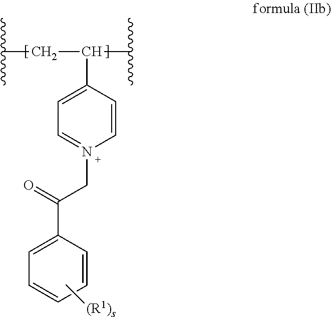

wherein in formula (II):
R¹OH; and
s is an integer from 0 to 3.

In another aspect, the disclosure describes a polymer comprising at least one moiety of formula (VIa):

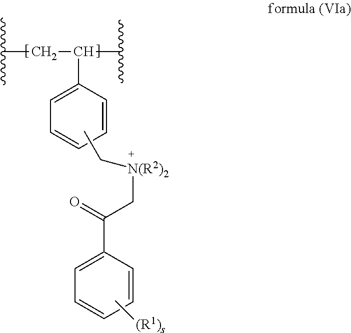

wherein in formula (VIa):
R¹ is OH;
each R² is independently optionally substituted alkyl; and
s is an integer from 0 to 3.

In another aspect, the disclosure describes a polyethylenimine (PEI) polymer comprising at least one moiety of formula (IXaa):

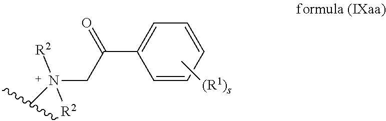

wherein in formula (IXaa):
R¹ is OH;
each R² is independently optionally substituted alkyl; and
s is an integer from 0 to 3.

In one aspect, the disclosure describes a compound of formula Ic):

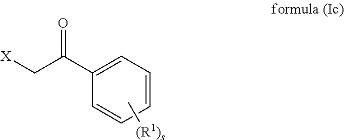

wherein in formula (Ic):
X is halogen, optionally substituted amine, azido, cyano, or —C(O)OR⁴; and
R¹ is OH;
R⁴ is selected from hydrogen and optionally substituted alkyl; and
s is an integer from 0 to 3.

In one aspect, the disclosure describes a method for preparing a polymer of the disclosure, the method comprising treating a precursor polymer with a compound of formula (Ic).

In one aspect, the disclosure describes a method for preparing a graftable substrate, the method comprising treating a substrate with a compound of formula (Ic).

In one aspect, the disclosure describes a compound of formula (XVa):

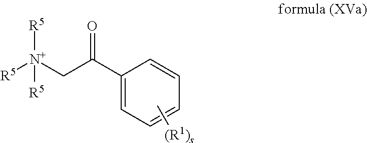

wherein in formula (XVa):
R¹ is OH;
each R⁵ is independently optionally substituted alkyl; and
s is an integer from 0 to 3.

In some embodiments, the polymer is a random copolymer. In some embodiments, the polymer comprises polyvinylpyridine or polyvinylbenzyl chloride. In one aspect, the disclosure describes compositions comprising at least one polymer of the disclosure. In one aspect, the disclosure describes compositions comprising at least one compound of the disclosure. In some embodiments, the composition is an antibacterial composition. In some embodiments, the composition is a biocidal composition. In some embodiments, the composition is an antiviral composition. In some embodiments, the composition is an antifungal composition. In some embodiments, the composition is an antiprotozoal composition.

In one aspect, the disclosure describes a solution comprising an alcohol and at least one polymer of the disclosure. In one aspect, the disclosure describes a solution comprising an alcohol and at least one compound of the disclosure. Any alcohol can be used, as understood by one of ordinary skill in the art. Non-limiting examples of alcohols include ethanol, methanol, n-propanol, isopropanol, t-butyl alcohol, and t-amyl alcohol. In some embodiments, the solution is an antibacterial solution. In some embodiments, the solution is a biocidal solution. In some embodiments, the solution is an antiviral solution. In some embodiments, the solution is an antifungal solution. In some embodiments, the solution is an antiprotozoal solution. In some embodiments, the solution is a ready-to-use solution for grafting.

In one aspect of the disclosure, methods for preparing the polymers described herein are provided. In some embodiments, the polymer is a polymer comprising at least one moiety of any one of formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXId), formula (XXII), formula (XXIIa), formula (XXIIb), formula (XXIIc), formula (XL), formula (XLa), formula (Ie), formula (IIb), formula (VIa), formula (IXaa), formula (Ic), and formula (XVa).

In one aspect of the disclosure, methods for preparing the compounds described herein are provided. In some embodiments, the compound is a compound of any one of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa).

In one aspect, the disclosure describes a method for preparing a polymer of the disclosure, the method comprising treating a precursor polymer with a compound of formula (Ib):

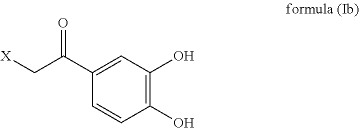

wherein in formula (Ib):
X is halogen, optionally substituted amine, azido, cyano, —SR⁴—, or —C(O)OR⁴; and
R⁴ is selected from hydrogen and optionally substituted alkyl.

In some embodiments, R⁴ is methyl or ethyl.

In some embodiments, the compound of formula (Ib) is selected from:

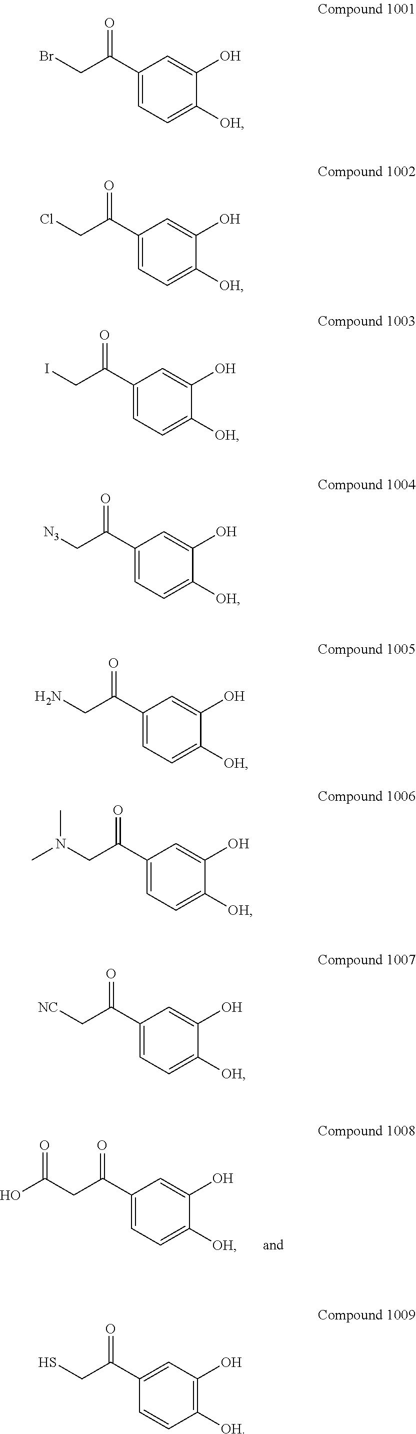

The precursor polymer is not limited, as would be understood by one of ordinary skill in the art. In some embodiments, the precursor polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide), poly(diallyldimethylammonium), or a C₃-C₂₂ alkyne. In one embodiment, the precursor polymer is fully methylated PEI.

In some embodiments, the polymer is selected from polyvinylpyridine (PVP) and polyethylenimine (PEI), and the compound of formula (Ib) is selected from Compound 1001, Compound 1002, and Compound 1003.

In some embodiments, the polymer is polyvinylbenzylchloride, and the compound of formula (Ib) is selected from Compound 1005 and Compound 1006.

In some embodiments, the polymer comprises a C₃-C₂₂ alkyne, and the compound of formula (Ib) is Compound 1004.

In some embodiments, the polymer comprises a cyano group, and the compound of formula (Ib) is Compound 1004.

In some embodiments, the polymer comprises a carboxyl group, and the compound of formula (Ib) is Compound 1005.

In some embodiments, the polymer comprises an azido group, and the compound of formula (Ib) is Compound 1007.

In some embodiments, the polymer comprises an amino group, and the compound of formula (Ib) is Compound 1008.

In some embodiments, the polymer comprises a thio group, and the compound of formula (Ib) is Compound 1009.

In some embodiments, the method further comprises treating the precursor polymer with the compound of formula (Ib) in a solvent. In some embodiments, the solvent is an alcohol. Non-limiting examples of alcohols include ethanol, methanol, n-propanol, isopropanol, t-butyl alcohol, and t-amyl alcohol.

In some embodiments, the method further comprises treating the precursor polymer with at least one optionally substituted C₄-C₂₂ alkyl halide. In some embodiments, the C₄-C₂₂ alkyl halide is a C₄ alkyl halide In some embodiments, the C₄-C₂₂ alkyl halide is a C₆ alkyl halide In some embodiments, the C₄-C₂₂ alkyl halide is a C₁₀ alkyl halide.

In another aspect, the disclosure describes a method for preparing polymers disclosed herein. In one aspect, the disclosure describes a method for preparing a polymer comprising treating a precursor polymer with a compound of formula (XIa):

Y—(CH₂)ᵥSi(R³)₃ formula (XIa)

wherein in formula (XIa):
Y is halogen, optionally substituted amine, cyano, azido, or —C(O)OR⁴;
each R³ is independently optionally substituted optionally substituted alkoxy;
R⁴ is selected from hydrogen and optionally substituted alkyl; and
v is an integer from 3 to 10.

In some embodiments, R³ is methoxy. In some embodiments, R⁴ is methyl or ethyl. In some embodiments, v is 3. In some embodiments, Y is

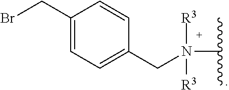

In some embodiments, the compound of formula (XIa) is selected from I(CH₂)₃Si(OMe)₃, N₃(CH₂)₃Si(OMe)₃, H₂N(CH₂)₃Si(OMe)₃, or (CH₃)₂N(CH₂)₃Si(OMe)₃.

In another aspect, the disclosure describes a method for preparing polymers disclosed herein. In one aspect, the disclosure describes a method for preparing a polymer comprising treating a precursor polymer with a compound of any one of formula (XVII), formula (XVIII), formula (XIX), or formula (XX).

The precursor polymer is not limited, as would be understood by one of ordinary skill in the art. In some embodiments, the precursor polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide), poly(diallyldimethylammonium), or a C₃-C₂₂ alkyne. In one embodiment, the precursor polymer is fully methylated PEI.

In some embodiments, the polymer is selected from polyvinylpyridine (PVP) and polyethylenimine (PEI), and the compound of formula (XIa) is I(CH₂)₃Si(OMe)₃.

In some embodiments, the polymer is polyvinylbenzylchloride, and the compound of formula (XIa) is selected from H₂N(CH₂)₃Si(OMe)₃ and (CH₃)₂N(CH₂)₃Si(OMe)₃.

In some embodiments, the polymer comprises a C₃-C₂₂ alkyne, and the compound of formula (XIa) is N₃(CH₂)₃Si(OMe)₃.

In some embodiments, the polymer comprises a cyano group, and the compound of formula (XIa) is N₃(CH₂)₃Si(OMe)₃.

In some embodiments, the polymer comprises a carboxyl group, and the compound of formula (XIa) is H₂N(CH₂)₃Si(OMe)₃.

In some embodiments, the method further comprises treating the precursor polymer with the compound of formula (XIa) in a solvent. In some embodiments, the solvent is an alcohol. Non-limiting examples of alcohols include ethanol, methanol, n-propanol, isopropanol, t-butyl alcohol, and t-amyl alcohol.

In some embodiments, the method further comprises treating the precursor polymer with at least one optionally substituted C₄-C₂₂ alkyl halide. In some embodiments, the C₄-C₂₂ alkyl halide is a C₁₀ alkyl halide.

In another aspect, the disclosure describes a method for preparing a compound of the disclosure. In some embodiments, the method comprising treating (R⁵)₃N with a compound of formula (Ib), wherein each R⁵ is independently optionally substituted alkyl:

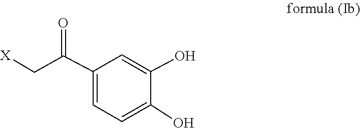

wherein in formula (Ib):
X is halogen;
and wherein in (R⁵)₃N, each R⁵ is independently optionally substituted alkyl.

In some embodiments, the compound of formula (Ib) is selected from:

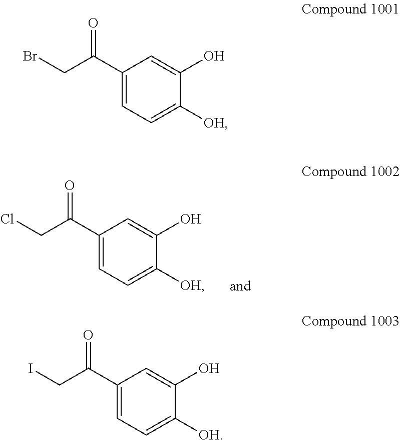 [SMILES: O=C(CBr)c1ccc(O)c(O)c1 ; O=C(CCl)c1ccc(O)c(O)c1 ; O=C(CI)c1ccc(O)c(O)c1]

### Surfaces

The disclosure provides in one aspect a surface grafted to a graftable substrate of the disclosure.

In one aspect of the disclosure, a compound of the disclosure is grafted onto a surface. In some embodiments, the compound is a compound of any one of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa). In some embodiments, the compound is a compound of any one of formula (Ib), formula (XV), formula (XVI), or formula (XVa). In some embodiments, the compound is a compound of any one of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa).

In one aspect of the disclosure, a polymer of the disclosure is grafted onto a surface. In some embodiments, the polymer is a polymer comprising at least one moiety of any one of formula (I), formula (Ia), formula (II), formula (III), formula (IV), formula (V), formula (VI), formula (VII), formula (VIII), formula (IXa), formula (IXb), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (Ie), formula (IIb), formula (VIa), formula (IXaa), formula (Ic), formula (XVa), formula (XL), or formula (XLa). In some embodiments, the polymer is a polymer comprising at least one moiety of any one of formula (I), formula (Ia), formula (II), formula (IV), formula (V), formula (VII), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), or formula (XVa). In some embodiments, the polymer is a polymer comprising at least one moiety of any one of formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), or formula (XLa).

When grafted, functional groups attached to the surface are covalently linked to moieties of the polymer or compound, such as catechol or silyl moieties. Grafting provides a stronger interaction between the surface and the polymer or compound than coating because in a coating, the interaction between the surface and the coated substrate is limited to electrostatic interactions rather than covalent linkages. Scheme 2 below shows an example of a polymer and compound of the disclosure grafted to a hydroxylated surface:

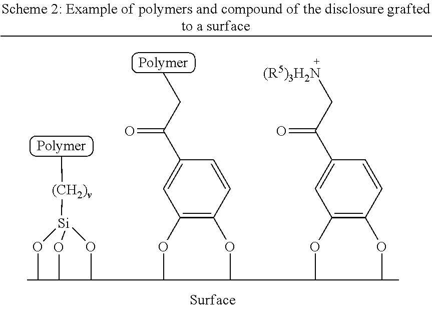

Any surface is contemplated by the disclosure, as understood by one of ordinary skill in the art. In a non-limiting example, the surface comprises a nanoparticle. Any nanoparticle is contemplated by the invention, as would be understood by one of ordinary skill in the art. In a non-limiting example, nanoparticles comprise any of Fe, Al, Cu, Zn, Mg, Mn, or other metal atoms. Non-limiting examples of surfaces include metals such as titanium and titanium alloys, iron, and steel; metal oxides; ceramics; polymers such as polyethylene (low and high reticulation for use in biomedical implants, after prior plasma activation), teflon (after prior plasma activation), polyethylene terephthalate (after prior plasma activation), and polypropylene (low and high density, after prior plasma activation), silicones, rubbers, latex, plastics, polyanhydrides, polyesters, polyorthoesters, polyamides, polyacrylonitrile, polyurethanes, polyethylene, polytetrafluoroethylene, polyethylenetetraphthalate and polyphazenes; paper; leather; textiles or textile materials such as cotton, jute, linen, hemp, wool, animals hair and silk, synthetic fabrics such as nylon and polyester; textile material includes fibers comprising fiber material such as acrylic polymers, acrylate polymers, aramid polymers, cellulosic materials, cotton, nylon, polyolefins, polyester, polyamide, polypropylene, rayon, wool, spandex, silk, and viscose; silicon; wood; glass; all cellulosic compounds; and gels and fluids not normally found within the human body. See, for example, US 2005/0249695, U.S. Pat. No. 8,475,782, US 2007/0292486, U.S. Pat. Nos. 4,282,366, 4,394,378, DE 2222997, DE 2229580, DE 2408192, GB 882067, all of which are incorporated by reference herein in their entireties.

In some embodiments, the surface comprises labile hydrogen atoms like thiols, amines or hydroxyl groups. In some embodiments, the surface comprises radical groups. In some embodiments, the surface comprises hydroxyl groups. In some embodiments, the surface is naturally hydroxylated. Examples of naturally hydroxylated surfaces include, but are not limited to, cotton, linen, leather, paper, cardboard, and wood. Some surfaces do not naturally contain such labile hydrogen atoms, and the labile hydrogen atoms have to be generated in situ, by using standard activation methods as would be understood by one of ordinary skill in the art. Non-limiting methods of activation include treatment with acid, oxidant treatment, plasma treatment, and UV/ozone treatment. Non-limiting examples of substances where hydroxylation occurred through activation include plastics, silicone, glass, and metals. In some embodiments, the surface is activated to produce hydroxyl groups.

In some embodiments, the cationic surface density of surfaces grafted with polymers and compounds disclosed herein is measured. The cationic surface density is a measurement of the charge density of quaternary ammonium compounds present on the surface, and a cationic surface density above a certain threshold indicates that bacterial death will occur quickly, as would be understood by one of ordinary skill in the art. In some embodiments, the cationic surface density is between about 10¹⁴/cm² and about 10²⁰/cm². In some embodiments, the cationic surface density is between about 10¹⁵/cm² and about 10¹⁷/cm². In some embodiments, the cationic surface density is greater than about 10¹⁵/cm².

In another aspect of the disclosure, the compounds, polymers, and/or graftable substrates of the disclosure can be used to change physical surface properties of materials. In a non-limiting example, compounds, polymers, and/or graftable substrates of the disclosure can modulate the hydrophilicity or hydrophobicity of a surface by varying the length of the lateral chain of the catechol or silane moiety. For example, alkyl chains more than four carbons in length (such as C₅-C₂₂ alkyl chains) can be added to the compounds and polymers of the disclosure, thereby increasing their hydrophobicity. In a non-limiting example, a compounds, polymers, and/or graftable substrates comprises quaternary nitrogens, wherein each quaternary nitrogen comprises either a moiety of the disclosure or a C₅-C₂₂ alkyl chain. After grafting, this increased hydrophobicity protects the grafted surfaces (such as wood, metals, plastics, textiles, and leather) by making them water-repellent to protect the surfaces from mold and mildew. In one embodiment, C₄-C₁₂ alkyl chains improve the biocidal properties of the compounds, polymers, and/or graftable substrates. In one embodiment, alkyl chains of C13 or greater increase the hydrophobicity of the compounds, polymers, graftable substrates, and/or graftable composition

In one aspect of the disclosure, compounds of the disclosure can be covalently attached to polymers comprising COOH and/or NH₂ moieties through an amidation reaction. The resulting polymer can then be directly grafted to surfaces without employing complex multi-step reactions.

### Grafting

In one aspect of the disclosure, methods for grafting graftable substrates of the disclosure are provided.

In one aspect of the disclosure, methods for grafting polymers are provided. In some embodiments, the polymer is a polymer comprising at least one moiety of any one of formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXId), formula (XXII), formula (XXIIa), formula (XXIIb), formula (XXIIc), formula (XL), formula (XLa), formula (Ie), formula (IIb), formula (VIa), formula (IXaa), formula (Ic), or formula (XVa).

In one aspect of the disclosure, methods for grafting polymers are provided. In some embodiments, the polymer is a polymer comprising at least one moiety of any one of formula (I), formula (Ia), formula (II), formula (III), formula (IV), formula (V), formula (VI), formula (VI), formula (VII), formula (VIII), formula (IXa), formula (IXb), formula (IXc), formula (IXd), formula (Ie), formula (IIb), formula (VIa), formula (IXa), or formula (Ic). In some embodiments, the polymer is a polymer comprising at least one moiety of any one of formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa).

In one aspect of the disclosure, methods for grafting compounds are provided. In some embodiments, the compound is a compound of any one of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa). In some embodiments, the compound is a compound of any one of formula (Ib), formula (XV), formula (XVI), or formula (XVa). In some embodiments, the compound is a compound of any one of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa).

In one aspect of the disclosure, methods for grafting compounds are provided. In some embodiments, the compound is a compound of any one of formula (XV), formula (XVI), or formula (XVa).

In one aspect, the disclosure describes methods for grafting a substrate onto a surface. In some embodiments, the method includes depositing a graftable substrate of the disclosure onto the surface; and heating the surface for a period of time. In some embodiments, the graftable substrate is deposited by spraying, dip coating, or spin-coating. In some embodiments, the graftable substrate is deposited in a solvent comprising an alcohol selected from ethanol, methanol, n-propanol, isopropanol, t-butyl alcohol, and t-amyl alcohol.

In one aspect, the disclosure describes methods of grafting a polymer onto a surface, the method comprising depositing a polymer of the disclosure onto the surface; and heating the surface for a period of time. In some embodiments, the polymer is deposited in a solvent. In some embodiments, the solvent is an alcohol. In some embodiments, the solvent is water. Non-limiting examples of alcohols include ethanol, methanol, n-propanol, isopropanol, t-butyl alcohol, and t-amyl alcohol.

In another aspect, the disclosure describes methods of grafting a compound onto a surface, the method comprising depositing a compound of the disclosure onto the surface; and heating the surface for a period of time. In some embodiments, the compound is deposited in a solvent. In some embodiments, the solvent is an alcohol. In some embodiments, the solvent is water. Non-limiting examples of alcohols include ethanol, methanol, n-propanol, isopropanol, t-butyl alcohol, and t-amyl alcohol.

Any method of deposition is contemplated for use herein, as would be understood by one of ordinary skill in the art. Non-limiting examples of methods of deposition include spraying, dip coating, or spin-coating.

Any temperature suitable for grafting is contemplated for use herein, as would be understood by one of ordinary skill in the art. In some embodiments, the surface is heated to a temperature ranging from about 50° C. to about 120° C. In some embodiments, the surface is heated to a temperature of about 110° C.

As would be understood by one of ordinary skill in the art, any temperature suitable for grafting is contemplated for use herein. In some embodiments, the surface is heated for a period of time ranging from about 15 minutes to about 12 hours. In some embodiments, the surface is heated for a period of time ranging from about 6 hours to about 12 hours. In some embodiments, the surface is heated for a period of time ranging from about 15 minutes to about 90 minutes. In some embodiments, the surface is heated for a period of time ranging from about 30 minutes to about 60 minutes. In some embodiments, if the polymer or compounds to be grafted comprises a catechol moiety, the surface is heated for a period of time ranging from about 15 minutes to about 12 hours.

In some embodiments, the method further comprises washing the surface with a solvent. Non-limiting examples of solvents that can be used for washing include ether.

In some embodiments, the method further comprises sonicating the surface. Sonication can be performed for periods of time including, but not limited to, 5 minutes, 15 minutes, or 30 minutes. In some embodiments, the surface is sonicated while in a solvent. In non-limiting examples, solvents useful for sonication include acetone, ethanol, and distilled water.

In some embodiments, the surface is activated prior to grafting. Non-limiting examples of activation include plasma activation, acid activation, or UV/ozone activation.

### Durable Coatings

In one aspect, the disclosure provides surface coatings comprising one or more polymers, substrates, and/or compounds of the disclosure having that are long-lasting, resistant to minor or moderate abrasion, and have durable self-cleaning properties.

In one aspect, the coatings are prepared by the sequential deposition of a sol-gel and a biocidal polymer solution. In some embodiments, the sol-gel comprises a mixture of an acid and a sol. In some embodiments, the biocidal polymer solution comprises one or more polymers, substrates, and/or compounds of the disclosure. In a non-limiting example, the sequential deposition of a titanium anatase sol-gel and an alcoholic biocidal coating solution comprising one or more polymers, substrates, and/or compounds of the disclosure, such as a mixture of a random catechol copolymer such as catecholacetyl-coalkyl PVP and alkyl(4-catecholacetyl)dimethylammonium chloride, provides durable, long-lasting coatings that resisted immersion in various solvents, water, and abrasion.

In some embodiments, the sol-gel comprising the mixture of the acid and the sol is deposited first, followed by the biocidal polymer solution comprising one or more polymers, substrates, and/or compounds of the disclosure. In some embodiments, the biocidal polymers solution comprising one or more polymers, substrates, and/or compound of the disclosures is deposited first, followed by the sol-gel comprising the mixture of the acid and the sol. In some embodiments, the sol comprises a source of titanium oxide. In some embodiments, the sol-gel comprising the mixture of the acid and the sol comprises a mixture of peroxo-modified anatase sol and peroxo titanic acid. In some embodiments, the sol-gel comprising the mixture of the acid and the sol is an aqueous solution. In some embodiments, the sol-gel comprising the mixture of the acid and the sol is diluted in an aqueous solution. In some embodiments, the solution comprising one or more polymers, substrates, and/or compounds of the disclosure is an alcoholic solution. In some embodiments, the biocidal polymer solution comprising one or more polymers, substrates, and/or compounds of the disclosure comprises ethanol.

In one aspect, the disclosure provides a method of preparing a coating. In some embodiments, the method comprises depositing a sol-gel, and depositing a biocidal polymer solution. In some embodiments, the method comprises depositing a sol-gel on a surface, and depositing a biocidal polymer solution. In some embodiments, the method comprises depositing a biocidal polymer solution on a surface, and depositing a sol-gel. In some embodiments, the sol-gel is deposited first, followed by the biocidal polymer solution. In some embodiments, the sol-gel forms a first layer, and the biocidal polymer solution forms a second layer. In some embodiments, the biocidal polymer solution is deposited first, followed by the sol-gel. In some embodiments, the biocidal polymer solution forms a first layer, and the sol-gel forms a second layer. In some embodiments, the second layer is in contact with the first layer. In some embodiments the sol-gel comprises a mixture of an acid and a sol. In some embodiments the sol-gel comprises a mixture of an acid and a sol, and the biocidal coating solution comprises one or more polymers, substrates, and/or compounds of the disclosure. In some embodiments, the acid is peroxo titanic acid. In some embodiments, the sol comprises a peroxo-modified anatase sol. In some embodiments, the sol-gel comprises a mixture of peroxo-modified anatase sol and peroxo titanic acid. In some embodiments, the sol-gel is an aqueous solution. In some embodiments, the sol-gel is diluted in an aqueous solution. In some embodiments, the biocidal polymer solution is an alcoholic solution. In some embodiments, the biocidal polymer solution comprises ethanol. In some embodiments, the first layer comprises metal oxide nanoparticles. In some embodiments, the first layer comprises titanium oxide nanoparticles. In some embodiments, the second layer comprises metal oxide nanoparticles. In some embodiments, the second layer comprises titanium oxide nanoparticles.

In one aspect, the disclosure provides a method of preparing a coating. In some embodiments, the method comprises depositing a first solution on a surface, and depositing a second solution. In some embodiments, the first solution forms a first layer, and the second solution forms a second layer. In some embodiments, the first solution and/or the second solution is a sol-gel. In some embodiments, the second layer is in contact with the first layer. In some embodiments the first solution comprises a sol-gel comprising a mixture of an acid and a sol, and the second solution comprises a biocidal coating solution comprising one or more polymers, substrates, and/or compounds of the disclosure. In some embodiments the first solution comprises a biocidal coating solution comprising one or more polymers, substrates, and/or compounds of the disclosure, and the second solution comprises a sol-gel comprising a mixture of an acid and a sol. In some embodiments, the sol-gel comprising the mixture of the acid and the sol comprises a mixture of peroxo-modified anatase sol and peroxo titanic acid. In some embodiments, the sol-gel comprising the mixture of the acid and the sol is an aqueous solution. In some embodiments, the sol-gel comprising the mixture of the acid and the sol is diluted in an aqueous solution. In some embodiments, the solution comprising one or more polymers, substrates, and/or compounds of the disclosure is an alcoholic solution. In some embodiments, the biocidal coating solution comprising one or more polymers, substrates, and/or compounds of the disclosure comprises ethanol. In some embodiments, the first layer comprises metal oxide nanoparticles. In some embodiments, the first layer comprises titanium oxide nanoparticles. In some embodiments, the second layer comprises metal oxide nanoparticles. In some embodiments, the second layer comprises titanium oxide nanoparticles.

In some embodiments, the method comprises waiting a suitable period of time for the first solution to dry. In some embodiments, the method comprises waiting a suitable period of time for the second solution to dry. In some embodiments, the method comprises waiting a suitable period of time for the sol-gel to dry. In some embodiments, the method comprises waiting a suitable period of time for the biocidal polymer solution to dry. In some embodiments, a suitable period of time for the first solution and/or second solution and/or sol-gel and/or biocidal polymer solution to dry ranges from about 1 minute to about 12 hours, about 1 minute to about 15 minutes, about 5 minutes to about 10 minutes, about 6 hours to about 12 hours, or about 3 hours to about 6 hours. In some embodiments, the drying is performed at a temperature in a range from about 0° C. to about 250° C., about 25° C. to about 100° C., about 30° C. to about 75° C. or about 20° C. to about 30° C. In some embodiments, the drying is performed at room temperature, above room temperature, about 20° C., about 25° C., about 30° C., about 50° C., about 75° C., about 100° C., about 125° C., about 150° C., about 175° C., or about 200° C. In some embodiments, waiting a suitable period of time for the sol-gel and/or the sol-gel comprising the mixture of the acid and the sol to dry provides a plurality of metal oxide nanoparticles substantially in contact with a surface. In some embodiments, the metal oxide nanoparticles are titanium oxide nanoparticles. In some embodiments, the one or more polymers, substrates, and/or compounds of the disclosure are grafted onto the surface of one or more of metal oxide nanoparticles.

In one aspect, the disclosure provides a coating comprising metal oxide nanoparticles and one or more one or more polymers, substrates, and/or compounds of the disclosure. one or more one or more polymers, substrates, and/or compounds of the disclosure In one aspect, the disclosure provides a coating comprising metal oxide nanoparticles and one or more compounds of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), formula (XXa), formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XXI), formula (XXIa), formula (XXIb), formula (XXII), formula (XXIIa), formula (XXIIb), formula (XXIIc), formula (XL), formula (XLa), formula (Ie), formula (IIb), formula (VIa), formula (IXaa), formula (Ic), and/or formula (XVa). In some embodiments, the coating comprises metal oxide nanoparticles and one or more compounds of formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa). In some embodiments, the coating comprises metal oxide nanoparticles and one or more compounds of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), formula (XLa).

In some embodiments, a plurality of metal oxide nanoparticles are substantially in contact with a surface. In some embodiments, the one or more one or more polymers, substrates, and/or compounds of the disclosure are grafted on the surface of one or more metal oxide nanoparticles.

In one aspect, the coating comprises a first layer in contact with a surface, and a second layer in contact with the first layer. In some embodiments, the first layer comprises metal oxide nanoparticles, and the second layer comprises one or more one or more polymers, substrates, and/or compounds of the disclosure. In some embodiments, the first layer comprises one or more one or more polymers, substrates, and/or compounds of the disclosure, and the second layer comprises metal oxide nanoparticles. In some embodiments, the metal oxide nanoparticles comprise titanium oxide nanoparticles. In some embodiments, the first layer comprises titanium oxide nanoparticles, and the second layer comprises one or more compounds of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), formula (XXa), formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXId), formula (XXII), formula (XXIIa), formula (XXIIb), formula (XXIIc), formula (XL), formula (XLa), formula (Ie), formula (IIb), formula (VIa), formula (IXaa), formula (Ic), and/or formula (XVa). In some embodiments, the first layer comprises titanium oxide nanoparticles, and the second layer comprises one or more compounds of formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa). In some embodiments, the first layer comprises titanium oxide nanoparticles, and the second layer comprises one or more compounds of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), formula (XLa). In some embodiments, the first layer comprises titanium oxide nanoparticles, and the second layer comprises a mixture of catecholacetyl-coalkyl-PVP and alkyl(4-catecholacetyl)dimethylammonium chloride.

In some embodiments, the coating comprises metal oxide nanoparticles. Non-limiting examples of metal oxide nanoparticles include titanium oxide, silicon oxide, zirconium oxide, zinc oxide, iron oxide, tungsten oxide, niobium oxide, lanthanum oxide, tin oxide, tantalum oxide, and one or more combinations thereof. In some embodiments, the metal oxide nanoparticles comprise titanium oxide nanoparticles.

In some embodiments, the coating comprising metal oxide nanoparticles is prepared using sol-gel application, as would be understood by one of ordinary skill in the art. In a non-limiting embodiment, a sol is combined with an aqueous solution of an acid to prepare a solution that can be deposited on a surface to provide a coating comprising metal oxide nanoparticles. In some embodiments, the sol comprises a source of metal oxide. Any source of metal oxide is contemplated by the present disclosure. Non-limiting examples of a source of metal oxide is anatase. In some embodiments, the anatase is peroxo-modified anatase. Any acid that can be combined with a source of metal oxide to prepare a sol is contemplated by the present disclosure. A non-limiting example of an acid is peroxo titanic acid. In some embodiments, the solution comprises a mixture of peroxo-modified anatase sol and peroxo titanic acid (titanium anatase). In some embodiments, the solution is an aqueous solution.

In some embodiments, the sol-gel comprises the acid and the sol comprising a source of metal oxide in a ratio of about 1:10 by weight/volume, about 1:5 by weight/volume, about 1:4 by weight/volume, about 1:3 by weight/volume, about 1:2 by weight/volume, about 1:1 by weight/volume, about 2:1 by weight/volume, about 3:1 by weight/volume, about 4:1 by weight/volume, about 5:1 by weight/volume, or about 10:1 by weight/volume.

In some embodiments, the sol-gel comprises a mixture of the sol and the acid in a range of about 0.1 wt % to about 2 wt %, about 0.5 wt % to about 1 wt %, or about 0.6 wt % to about 0.9 wt % by weight. In some embodiments, the sol-gel comprises a mixture of the sol and the acid of about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 20 wt %. In some embodiments, the sol-gel comprises a mixture of the sol and the acid of about 0.8 wt %. In some embodiments, the sol comprises a source of metal oxide. In some embodiments, the sol-gel comprises a mixture of peroxo-modified anatase sol and peroxo titanic acid (titanium anatase) of about 0.8% wt %. In some embodiments, the sol-gel is an aqueous solution.

In some embodiments, the coating of the disclosure comprises one or more of any polymers, substrates, and/or compounds of the disclosure. In some embodiments, the coating comprises a catechol moiety. In some embodiments, the durability of the coatings can be increased by the use of catechol moieties compared to other chemical moieties, while maintaining optimal efficiency. In a non-limiting example, mussel-inspired biomimetic strategies were developed in order to increase the bonding strength between coatings and surfaces by, for example, electrostatic interactions. The use of catecholamine was examined since catecholeamine is very sensitive to polymerization. In a non-limiting example, 4-chloroacetylcatechol and derivatives thereof were found to be useful as linkers to attach biocides to surfaces, either via electrostatic interactions or via covalent bonding. In some embodiments, the catechol moiety exhibited a more robust interaction with titanium nanoparticles compared with a silane moiety. Although not wishing to be bound by any particular theory, these results may be due to the Ti—O—C bond displaying a higher stability than the Ti—O—Si bond. In some embodiments, the combination of catechol moieties and titanium oxide nanoparticles was found to increase the durability of the biocidal coating. In some embodiments, the titanium nanoparticles of the coatings are able to interact with catechol moieties through Ti—O—C bonds. Also, most organosilanes are easily hydrolysable which makes their use difficult in wet or humid environments. In contrast, catechol-based compounds provide excellent adherence underwater.

In some embodiments, the coating comprises one or more compounds of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), formula (XXa), formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXId), formula (XXII), formula (XXIIa), formula (XXIIb), formula (XXIIc), formula (XL), formula (XLa), formula (Ie), formula (IIb), formula (VIa), formula (IXaa), formula (Ic), and formula (XVa). In some embodiments, the coating comprises one or more compounds of formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa). In some embodiments, the coating comprises one or more compounds of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), and formula (XLa).

In some embodiments, the coating comprises one or more compounds of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), formula (XXa), formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XXI), formula (XXIa), formula (XXIb), formula (XXII), formula (XXIIa), formula (XXIIb), formula (XXIIc), formula (XL), formula (XLa), formula (Ie), formula (IIb), formula (VIa), formula (IXaa), formula (Ic), and formula (XVa) and metal oxide nanoparticles. In some embodiments, the coating comprises one or more compounds of formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa) and metal oxide nanoparticles. In some embodiments, the coating comprises one or more compounds of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), and formula (XLa) and metal oxide nanoparticles.

In some embodiments, the biocidal coating solution comprising one or more polymers, substrates, and/or compounds of the disclosure is an alcohol solution. Any alcohol is contemplated by the disclosure. Non-limiting examples of alcohols include ethanol, methanol, n-propanol, isopropanol, t-butyl alcohol, and t-amyl alcohol. In some embodiments, the alcohol is ethanol. In some embodiments, the biocidal coating solution comprises one or more compounds of formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), formula (XXa), formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXId), formula (XXII), formula (XXIIa), formula (XXIIb), formula (XXIIc), formula (XL), formula (XLa), formula (Ie), formula (IIb), formula (VIa), formula (IXaa), formula (Ic), and formula (XVa). In some embodiments, the biocidal coating solution comprises one or more compounds of formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa). In some embodiments, the biocidal coating solution comprises one or more compounds of formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), formula (XLa).

In some embodiments, the biocidal coating solution comprises a mixture of two or more polymers, substrates, and/or compounds of different structures. In some embodiments, the biocidal coating solution comprises two polymers, substrates, and/or compounds of different structures in a ratio of about 1:10 by weight/volume, about 1:5 by weight/volume, about 1:4 by weight/volume, about 1:3 by weight/volume, about 1:2 by weight/volume, about 1:1 by weight/volume, about 2:1 by weight/volume, about 3:1 by weight/volume, about 4:1 by weight/volume, about 5:1 by weight/volume, or about 10:1 by weight/volume. In some embodiments, the biocidal coating solution comprises two polymers, substrates, and/or compounds of different structures in a ratio of about 1:1 by weight/volume.

In some embodiments, the biocidal coating solution comprises one or more polymers, substrates, and/or compounds in a range of about 0.1 wt % to about 2 wt %, about 0.5 wt % to about 1 wt %, or about 0.6 wt % to about 0.9 wt % by weight. In some embodiments, the biocidal coating solution comprises one or more polymers, substrates, and/or compounds at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 20 wt %. In some embodiments, the solution comprises one or more polymers, substrates, and/or compounds of different structures at about 1 wt %.

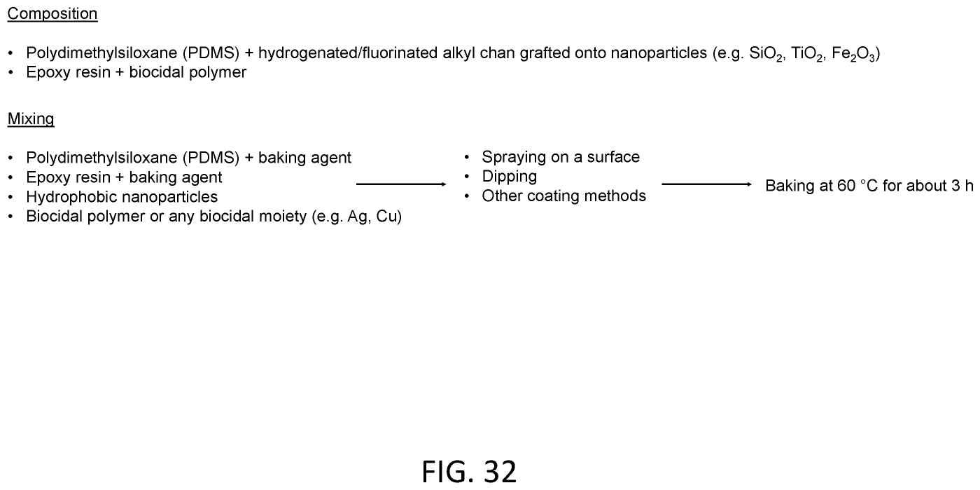

In one aspect, the disclosure provides a coating that is biocidal and hydrophobic, hydrophilic, and/or oleophobic. A non-limiting example of a method of preparing the coating is shown in FIG. 32.

In some embodiments, the coating comprises:
a) a siloxane-based polymer;
b) metal oxide nanoparticles;
c) an epoxy resin;
d) a biocidal moiety; and
e) one or more baking agents;
   wherein a plurality of the metal oxide nanoparticles are coated with a hydrophobic coating.

Any siloxane-based polymer is contemplated by the disclosure. Non-limiting examples of siloxane-based polymers include polydimethylsiloxane (PDMS), polyvinyl siloxane, hexamethyldisiloxane, hexamethylcyclotrisiloxane, octamethylcyclotetrasiloxane, dodecamethylcyclohexasiloxane, decamethylcyclopentasiloxane, octamethyltrisiloxane, decamethyltetrasiloxane, dodecamethylpentasiloxane, and tetradecamethylhexasiloxane. In some embodiments, the siloxane-based polymer is polydimethylsiloxane (PDMS).

In some embodiments, the coating comprises metal oxide nanoparticles. Non-limiting examples of metal oxide nanoparticles include titanium oxide, silicon oxide, zirconium oxide, zinc oxide, iron oxide, tungsten oxide, niobium oxide, lanthanum oxide, tin oxide, tantalum oxide, and one or more combinations thereof. In some embodiments, the metal oxide nanoparticles comprise titanium oxide nanoparticles. In some embodiments, a plurality of the metal oxide nanoparticles are substantially in contact with a surface.

In some embodiments, the hydrophobic coating comprises one or more hydrogenated and/or fluorinated alkyl chains, (hydrogenated n-alkyl chains between C12 and C22 or fluorinated n-alkyl chains between C₆ and C₁₂). In some embodiments, fluorinated n-alkyl the alkyl chain is C12 or shorter, since alkyl chains longer than C12 for fluorinated n-alkyl chains can exhibit poor solubility.

Any epoxy resin is contemplated by the disclosure. Non-limiting examples of epoxy resins include bisphenol A diglycidyl ether (DGEBA), diglycidyl ether bisphenol F (DGEBF), and Bisphenol E diglycidyl ether (DGEBE).

In one aspect, the biocidal moiety is one or more compounds selected from formula (Ib), formula (XV), formula (XVI), formula (XVa), formula (XVII), formula (XVIIa), formula (XVIIb), formula (XVIIc), formula (XVIII), formula (XIX), formula (XIXa), formula (XIXb), formula (XIXc), formula (XIXd), formula (XX), formula (XXa), formula (I), formula (Ia), formula (II), formula (III), formula (IV), formula (V), formula (VI), formula (VII), formula (VIII), formula (IXa), formula (IXb), formula (IXb1), formula (IXc), formula (IXd), formula (IXe), formula (IXe1), formula (IXf), formula (IXg), formula (IXh), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXId), formula (XXII), formula (XXIIa), formula (XXIIb), formula (XXIIc), formula (XL), formula (XLa), formula (Ie), formula (IIb), formula (VIa), formula (IXaa), formula (Ic), and formula (XVa). In one aspect, the biocidal moiety is one or more compounds selected from formula (Ib), formula (XV), formula (XVI), formula (XVa), formula (I), formula (Ia), formula (II), formula (IV), formula (V), formula (VI), formula (VIII), formula (IXa), formula (IXb), formula (IXc), formula (IXd), formula (IXe), formula (IXe1), formula (IXf), formula (IXg), formula (IXh), formula (Ie), formula (IIb), formula (VIa), formula (IXaa), formula (Ic), and formula (XVa). In one aspect, the biocidal moiety is one or more compounds selected from formula (XVII), formula (XVIIa), formula (XVIIb), formula (XVIIc), formula (XVIII), formula (XIX), formula (XIXa), formula (XIXb), formula (XIXc), formula (XIXd), formula (XX), or formula (XXa), formula (XI), formula (XII), formula (XIII), formula (XIV), formula (XIa), formula (XIb), formula (XIc), formula (XVIIa), formula (XVIIIa), formula (XVIIb), formula (XVIIc), formula (XVIId), formula (XVIIe), formula (XVIIf), formula (XVIIg), formula (XVIIh), formula (XVIIj), formula (XXI), formula (XXIa), formula (XXIb), formula (XXII), formula (XXIIa), formula (XXIIb), formula (XXIIc), formula (XXId), formula (XL), formula (XLa). In some embodiments, the biocidal moiety is a metal the exhibits biocidal properties. Non-limiting examples of metals useful within the disclosure include silver and copper.

Any baking agent (e.g. curing agent) is contemplated by the disclosure. Non-limiting examples of baking and/or curing agents include aliphatic amines, polyamides, cycloaliphatic amines, aromatic amines, anhydrides, imidazoles, and Lewis acids.

In some embodiments, the siloxane-based polymer, metal oxide nanoparticles, epoxy resin, biocidal moiety, and one or more baking agents are combined and mixed to form a mixture. In some embodiments, the mixture applied to a surface. In some embodiments, a baking agent is added to and mixed with the siloxane-based polymer before being combined with the mixture. In some embodiments, a baking agent is added to and mixed with the epoxy resin before being combined with the mixture.

In some embodiments, after the mixture is applied to a surface, the mixture is baked. In some embodiments, the mixture is baked at a temperature of about 40° C. to about 80° C., or about 50° C. to about 70° C. In some embodiments, the mixture is baked at a temperature of about 40° C., about 45° C., about 50° C., about 55° C., about 60° C., about 65° C., about 70° C., about 75° C., or about 80° C.

In some embodiments, the mixture is baked for a suitable time period for the coating to harden. In some embodiments, the mixture is baked for about 1 h to about 5 h, or about 2 h to about 4 h. In some embodiments, the mixture is baked for about 1 h, about 2 h, about 3 h, about 4 h, or about 5 h.

The coatings may be applied to the surface using any method known in the art, as would be understood by one of ordinary skill in the art. In some embodiments, the coating is applied using an electrostatic sprayer. The use of electrostatic sprayers has the advantage of providing a homogeneous deposition of microdroplets onto targeted surfaces. Electrostatic sprayers have gained significant attraction during the COVID-19 pandemic, especially as useful tools for large sanitation of public areas such as stadiums, hospitals, airports, etc. In contrast to the durable and long-lasting coatings of the disclosure, most currently used compounds that are sprayed (hydrogen peroxide, chlorhexidine, long chain quaternary ammonium compounds) present only transient efficacy and durability. In some embodiments, the coating is applied using a dipping process.

The coatings described herein may be applied to any surface. Non-limiting examples of surfaces include metals such as cobalt, cobalt-chrome alloys, aluminum, titanium and titanium alloys, iron, steel and stainless steel; metal oxides; ceramics; polymers such as polyethylene, Teflon, polyethylene terephthalate, and polypropylene, silicones, rubbers, latex, plastics, polyanhydrides, polyesters, polyorthoesters, polyamides, polyacrylonitrile, polyurethanes, polytetrafluoroethylene, polyethylenetetraphthalate and polyphazenes, leather, textiles or textile materials, synthetic fabrics such as nylon and polyester; textile material comprising fibers comprising fiber material such as acrylic polymers, acrylate polymers, aramid polymers, nylon, polyolefins, polyester, polyamide, polypropylene, rayon, spandex, silk, viscose, silicon, and glass. In a non-limiting embodiment, the durability of the coatings was found to be particularly efficacious when the compositions and formulations were coated onto metallic substrates. In some embodiments, the surface is a metallic surface. In some embodiments, the surface is activated and/or naturally hydroxylated.

### Conjugated Biomolecules

In one aspect, the disclosure provides biocidal moieties of the disclosure conjugated to biomolecules.

In one aspect, the disclosure provides a compound of formula (XL):

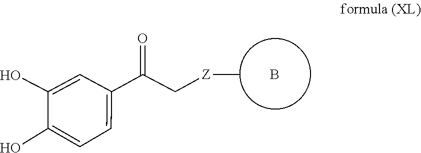

wherein in formula (XL).
Z is a single bond or a linking group; and
B is a biomolecule.

Any molecule with biological properties is contemplated by the present disclosure. In a non-limiting example, the biomolecule is a protein, enzyme, or peptide. In some embodiments, the biomolecules comprise a thiol group, such as a thiolated amino acid (e.g. cysteine). In some embodiments, the biomolecule comprises cysteine. In some embodiments, the biomolecule is cysteine. Non-limiting examples of biomolecules include bovine serum albumin, enzymes (oxidoreductases, transferases, hydrolases, lyases, ligases, and isomerases), polypeptides, antibodies, proteins including fluorescent proteins such as green fluorescent proteins (GFP), cerulean, mCherry, pericam, cameleons, pHluorins, EGFP (enhanced green fluorescent protein, clomeleon, halorhodopsins, channel rhodopsins, archaerhodopsins, mermaid, and GECI.

In some embodiments, the biomolecule comprises one or more detectable moieties. In some embodiments, the detectable moiety is one or more selected from a fluorescent moiety, a phosphorescent moiety, and a luminescent moiety. In some embodiments, the detectable moiety is a fluorescent moiety selected from a coumarin moiety, a fluorescein moiety, a rhodamine moiety, an acridine moiety, an indole moiety, an isoindole moiety, an indolizine moiety, a quinoline moiety, an isoquinoline moiety, a chromene moiety, a xanthene moiety, a naphthalene moiety, a pyrene moiety, an a bimane moiety.

In one aspect, the disclosure provides a compound of formula (XLa):

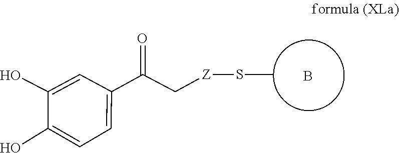

wherein in formula (XLa):
Z is a single bond or a linking group; and
B is a biomolecule.

In some embodiments, Z is a single bond. In some embodiments, Z is a linking group. The linking group Z may be any organic moiety, as would be understood by one of ordinary skill in the art. In some embodiments, the linking group is selected from one or more linking groups selected from optionally substituted heterocyclyl, optionally substituted thiol, optionally substituted alkyl, optionally substituted aryl, optionally substituted alkoxy, —S—, and —O—. In some embodiments, the optionally substituted heterocycle is selected from optionally substituted triazole and optionally substituted succinimide. In some embodiments, the succinimide is a disuccinimide. In some embodiments, the succiminime is substituted with a thiol group.

In some embodiments, Z is selected from

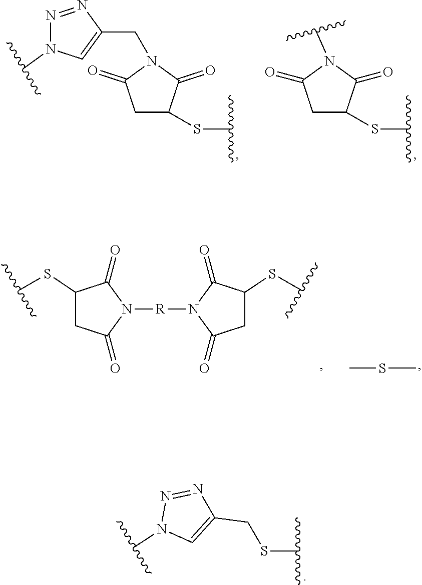

In some embodiments, Z is selected from a single bond,

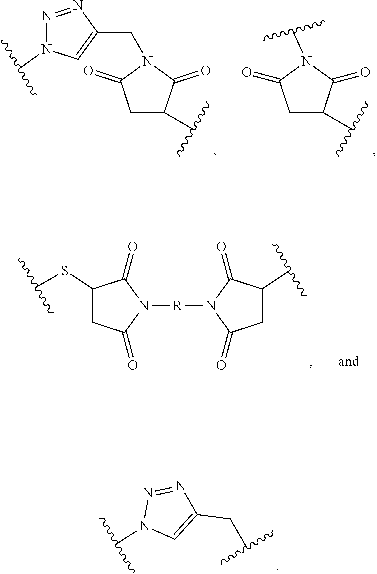

Any α, ω succinimide is contemplated by the present invention. Non-limiting examples of R include

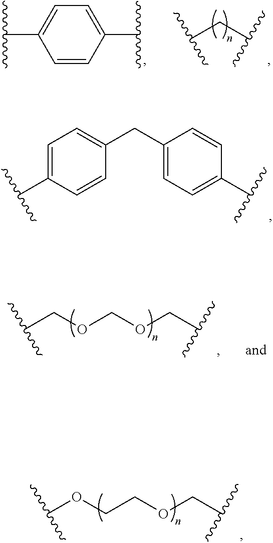

wherein is an integer from 1 to 5. In some embodiments, R is

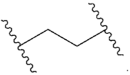 [SMILES: *CC*]

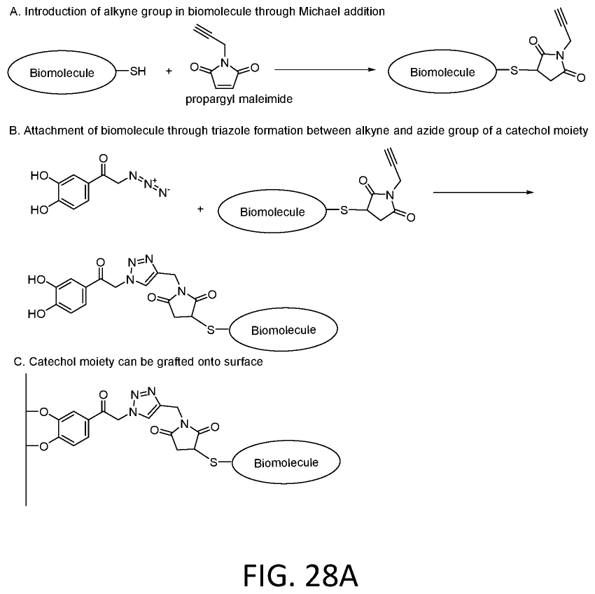

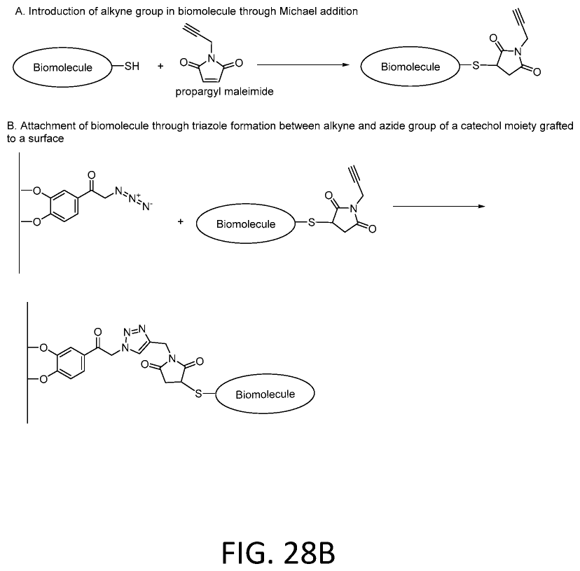

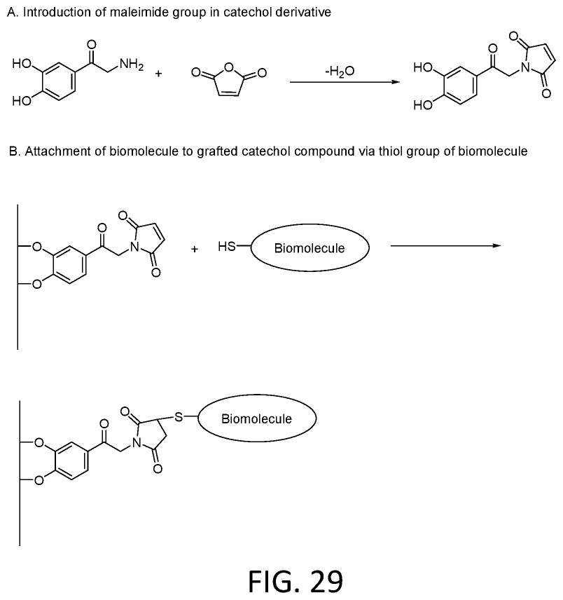

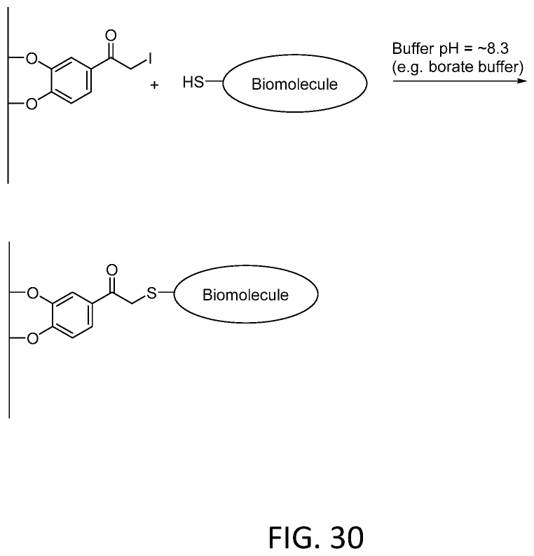

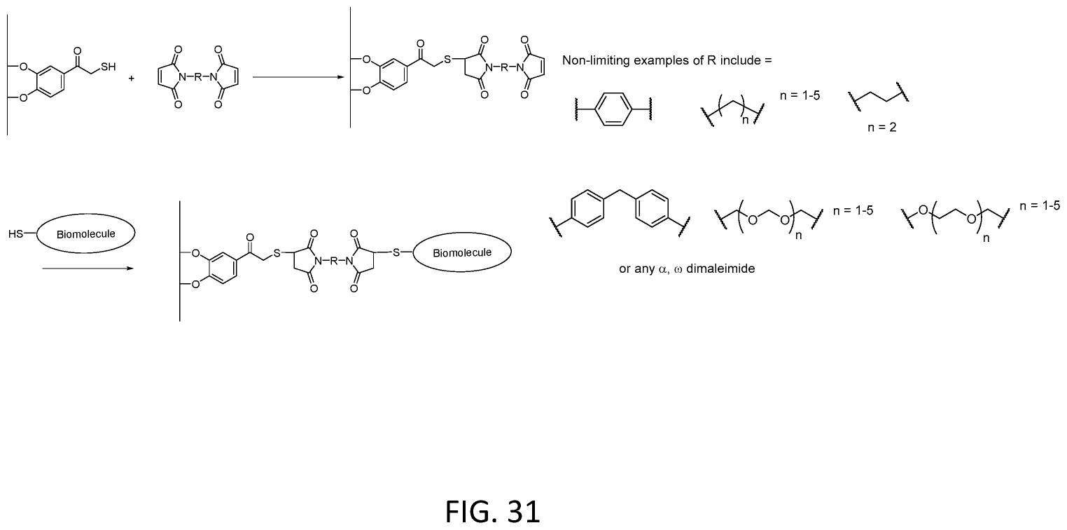

Non-limiting examples of methods for preparing compounds of formula (XL) and formula (XLa) are shown in FIG. 28-FIG. 31.

In some embodiments, the compounds of formula (XL) and/or formula (XLa) are grafted and/or applied onto surfaces to provide biocidal coatings that also comprise useful biomolecules. In some embodiments, the coatings are applied to biomedical applications such as medical devices, implants, contact lenses, catheters, and biosensors. In some embodiments, the coatings are useful as diagnostic agents.

### METHODS OF USE

In one aspect of the disclosure, methods for controlling the growth of at least one bacteria, fungi, protozoa, or virus are provided. In some embodiments, the method comprising grafting a graftable substrate of the disclosure on a surface. In some embodiments, the method comprises grafting a compound of the disclosure onto a surface. In some embodiments, the method comprises grafting a polymer and/or compound of the disclosure onto a surface. In some embodiments, the surface is activated prior to grafting.

In some embodiments, the bacteria is a gram-positive bacteria selected from M. tuberculosis (including multi drug resistant TB and extensively drug resistant TB), M bovis, M typhimurium, M bovis strain BCG, BCG substrains, M avium, M intracellulare, M africanum, M kansasii, M marinum, M ulcerans, M avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthraces, B. subtilis, Nocardia asteroides, and other Nocardia species, Streptococcus viridans group, Peptococcus species, Peptostreptococcus species, Actinomyces israelii and other Actinomyces species, Propionibacterium acnes, Clostridium tetani, Clostridium perfringens, Clostridium botulinum, other Clostridium species, and Enterococcus species.

In some embodiments, the bacteria is a gram-negative bacteria selected from Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species, Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, Escherichia hirae, and other Escherichia species, as well as other Enterobacteriacae, Burkholderia cepacia, Burkholderia pseudomallei, Francisella tularensis, Bacteroides fragilis, Fusobascterium nucleatum, Provetella species, Cowdria ruminantium, Klebsiella species, and Proteus species.

In some embodiments, the virus is selected from avian influenza, human immunodeficiency virus, herpex simplex virus, human respiratory syncytial virus, Middle East respiratory syndrome-related coronavirus (MERS-CoV), rhinovirus, polio, rotavirus, measles, Ebola, West Nile, yellow fever, Dengue fever, lassa, lymphocytic choriomeningitis, Junin, Machupo, guanarito, hantavirus, Rift Valley Fever, La Crosse, California encephalitis, Crimean-Congo, Marburg, Japanese Encephalitis, Kyasanur Forest, severe acute respiratory syndrome (SARS), severe acute respiratory syndrome coronavirus 2 (SARS-CoV-2), parainfluenza, and Pichinde viruses.

In another aspect, the disclosure describes a method of preventing digestion of cellulose by an organism. In some embodiments, the method comprising grafting a polymer or a compound of the disclosure onto a surface comprising cellulose. In some embodiments, surface comprises wood cellulose. In some embodiments, the organism is selected from a wood boring gribble, a shipworm, a woodlice, and a wood-boring insect. Non-limiting examples of wood-boring insects include termites, bark beetles, horntail larvae, moth larvae, beetles. In some embodiments, the insect is a xylophage. Non-limiting examples of xylophages include termites, bark beetles, horntail larvae, moth larvae, and beetles.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Preparation of Catechol Moieties

### 4-(Iodoacetyl)Catechol

4-(Chloroacetyl)catechol was treated with sodium iodide in acetone and heating to reflux for 3 hours to produce 4-(iodoacetyl)catechol. The reaction sequence is depicted in Scheme 3.

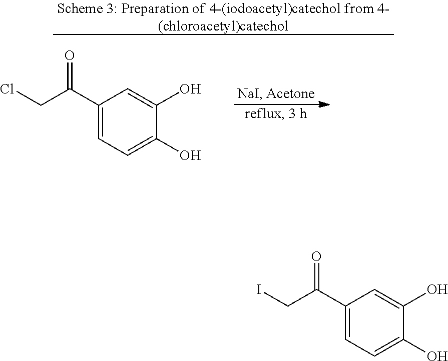 [SMILES: O=C(CCl)c1ccc(O)c(O)c1 ; "NaI, Acetone" ; "reflux, 3 h" ; O=C(CI)c1ccc(O)c(O)c1]

### 4-(Azidoacetyl)Catechol

4-(Chloroacetyl)catechol was treated with sodium azide in isopropanol and heated to reflux for 6 hours to produce 4-(azidoacetyl)catechol. The reaction sequence is depicted in Scheme 4.

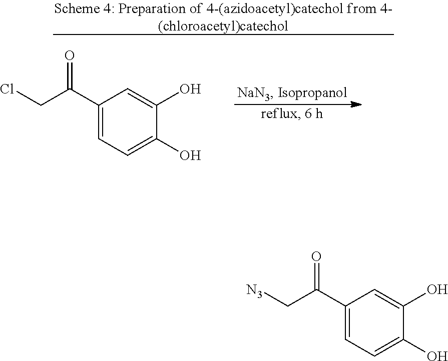 [SMILES: O=C(CCl)c1ccc(O)c(O)c1 ; "NaN3, Isopropanol" ; "reflux, 6 h" ; [N-]=[N+]=NCC(=O)c1ccc(O)c(O)c1]

In a non-limiting example, 4-(azidoacetyl)catechol is linked to an alkyne using click chemistry to produce, respectively, triazole or tetrazole linkages, as would be understood by one of ordinary skill in the art. Scheme 5 illustrates examples of linkages formed using click chemistry.

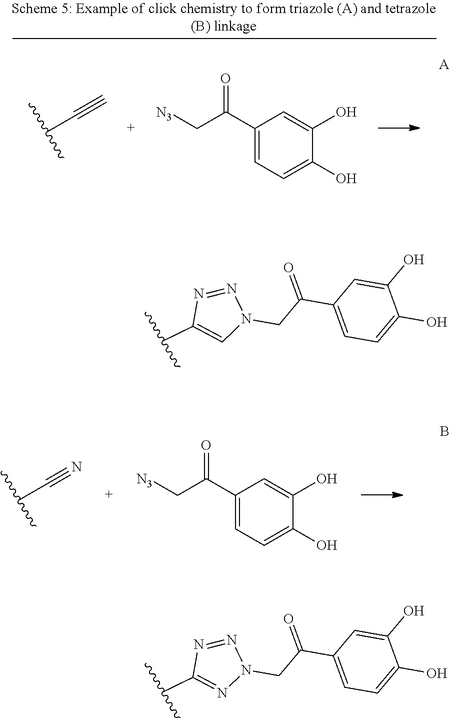

### 4-(Aminoacetyl)Catechol

4-(Azidoacetyl)catechol was treated with triphenylphospine in water to produce 4-(aminoacetyl)catechol. The reaction sequence is depicted in Scheme 6:

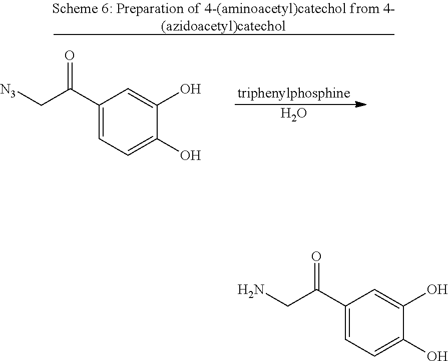 [SMILES: [N-]=[N+]=NCC(=O)c1ccc(O)c(O)c1 ; "triphenylphosphine" ; O ; NCC(=O)c1ccc(O)c(O)c1]

In a non-limiting example, 4-(aminooacetyl)catechol is linked to a carbonyl to form an amide, as would be understood by one of ordinary skill in the art. Scheme 7 illustrates an example of an amide linkage.

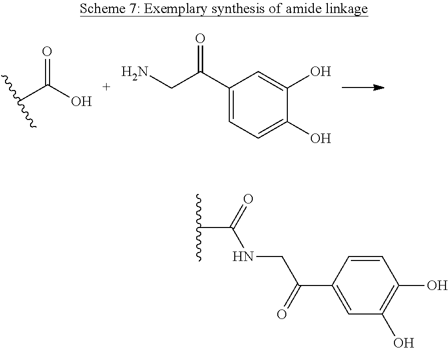

### 4-(Cyanoacetyl)Catechol

4-(Chloroacetyl)catechol was treated with potassium cyanide in dimethylformamide (DMF) to produce 4-(cyanoacetyl)catechol. The reaction sequence is depicted in Scheme 8:

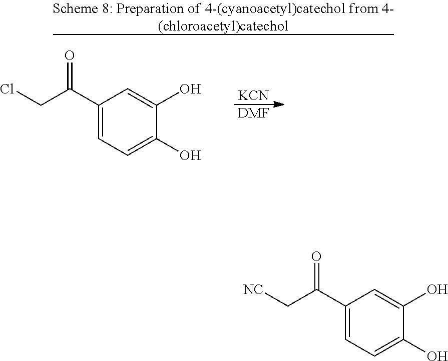

In a non-limiting example, 4-(cyanoacetyl)catechol is linked to an alkyne using click chemistry to produce a tetrazole linkage, as would be understood by one of ordinary skill in the art. Scheme 9 illustrates an example of a tetrazole linkage.

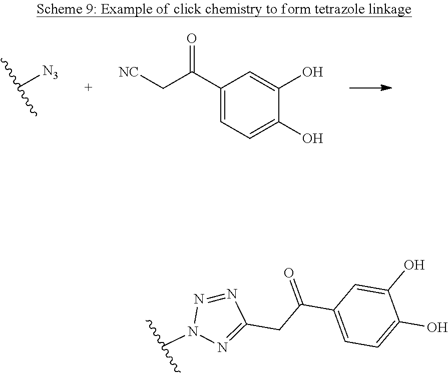

### 3-(3,4-Dihydroxybenzoyl) Propionic Acid

4-(Chloroacetyl)catechol was first treated with potassium cyanide in dimetylformamide (DMF) to form 4-(cyanoacetyl)catechol, followed by treatment with water to produce 3-(3,4-dihydroxybenzoyl) propionic acid. The reaction sequence is depicted in Scheme 10:

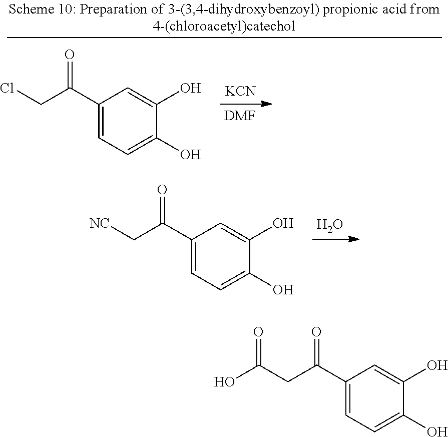

In a non-limiting example, 3-(3,4-dihydroxybenzoyl) propionic acid can be linked to an amine to form an amide, as would be understood by one of ordinary skill in the art. Scheme 11 illustrates an example of an amide linkage.

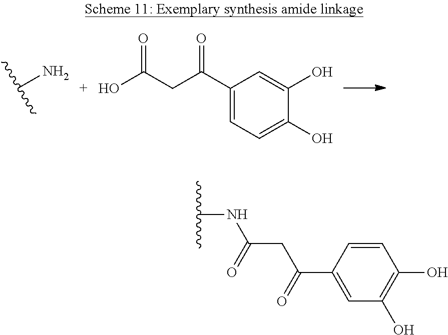

### 4-(Dimethylaminoacetyl)Catechol

4-(Iodoacetyl)catechol was treated with dimethylamine and potassium carbonate in tetrahydrofuran in the presence of potassium carbonate to produce 4-(dimethylaminoacetyl)catechol. The reaction sequence is depicted in Scheme 12A:

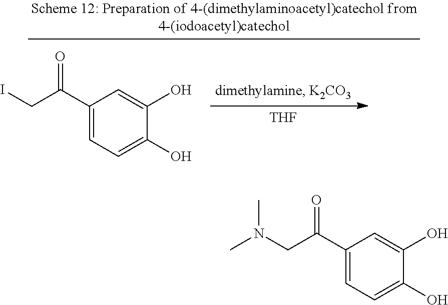

### Example 2: One-Pot Synthesis of Graftable Antibacterial Moieties

Surface grafting to confer antibacterial properties to high-touch surfaces can be achieved by the quaternization of long chain tertiary amines with halo-alkyl triethoxy or trimethoxysilanes. Even if this synthetic route provides a high yield of quaternary ammonium compounds, a small amount of volatile silane (<1%) still remains. This amount of silyl contaminant is potent enough to render spraying the quaternary ammonium compounds hazardous because of the particularly volatile properties of the silane compound. Reticulation in volume can cause serious damage to the eyes during the spraying procedure. Therefore, another process to graft these compositions on certain surfaces using a 4-haloacetylcatechol moiety was developed. In some embodiments, the 4-haloacetylcatechol moiety is treated with a dialkylaminoalkane in an alcohol (such as methanol and/or ethanol) and heated to reflux for 24 h. The general reaction sequence is depicted in Scheme 13A:

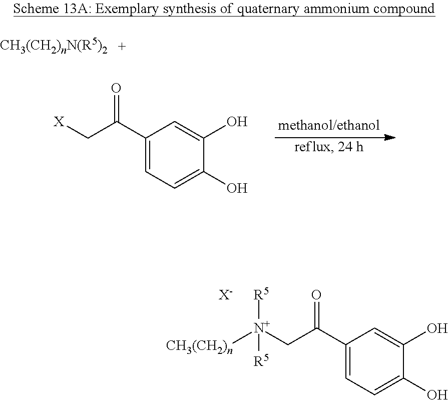

In Scheme 13A above, each R⁵ is independently optionally substituted alkyl; X is C1, Br, or I, and n is an integer from 3 to 21.

This method of preparation does not result in the formation of byproducts. The 4-haloacetylcatechol moiety product is soluble in alcohols such as ethanol/methanol and in water, and it is ready to graft on a variety of surfaces, including hydroxylated surfaces (hydroxylation can be naturally-occurring on the surface or achieved by activation). When this moiety is deposited using methods such as spraying methods, the 4-haloacetylcatechol moiety does not present the same risks as the volatile silanes because the 4-haloacetylcatechol moiety is generally a solid at ambient temperature.

Scheme 13B below describes an alternative approach to preparing a quaternary ammonium catechol compound using 4-(dimethylaminoacetyl)catechol. 4-(dimethylaminoacetyl)catechol is treated with an alkyl halide (in a non-limiting example, the alkyl halide comprises a C₁₆-C₂₀ alkyl chain) to provide a quaternary 4-(alkyldimethylaminoacetyl)catechol compound.

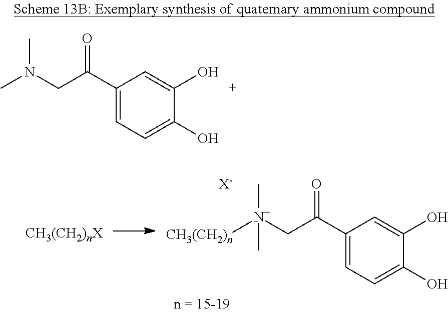

### Example 3: Synthesis of a Ready-to-Graft Catechol-Based Quaternary Ammonium Moieties

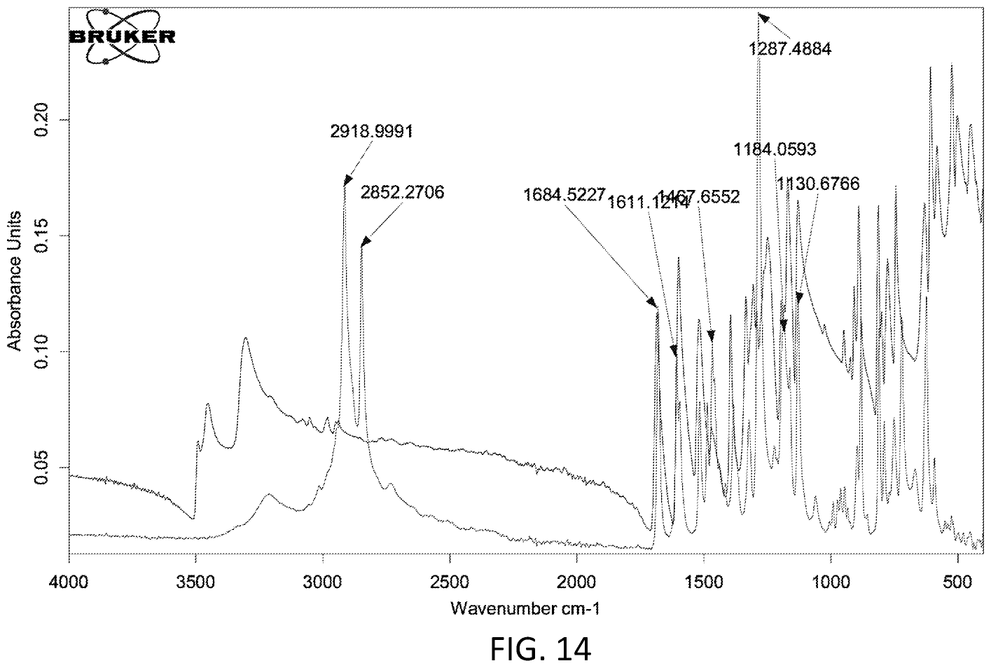

1 gram of N, N dimethyloctadecylamine 89% (Mw: 297.57 g/mol; 1 eq) was reacted with 620 mg of 4-chloroacetylcatechol (Mw: 186.59 g/mol; 1 eq) in boiling isopropyl alcohol for 24 hours to obtain the product octadecyl(4-catecholacetyl)dimethylammonium chloride (ODMcat) (90% yield). FIG. 14 shows the IR spectrum of octadecyl(4-catecholacetyl)dimethylammonium chloride compared to 4-chloroacetylcatechol. The reaction sequence is depicted in Scheme 14:

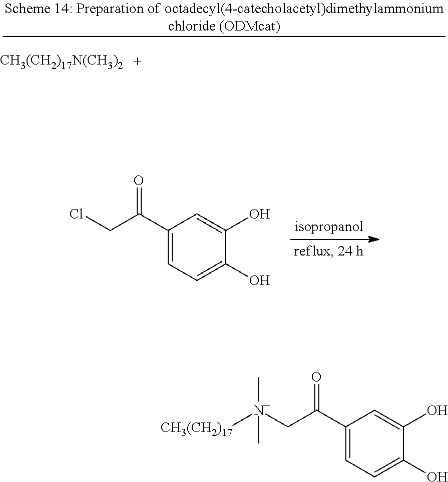

### Example 4: Grafting on Filter Paper

### Depositing and Grafting:

A 1 cm² piece of filter paper was impregnated with a solution of ODMcat in isopropanol and baked in an oven at 110° C. for one hour. Subsequently the sample was rinsed with ethanol and sonicated in the same solvent for 20 min in order to eliminate excess reagent.

### Visual Fluorescein Test

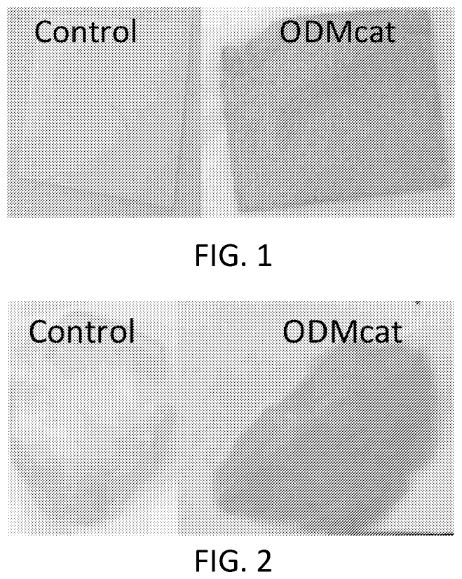

Samples of the impregnated filter paper were transferred to a 1% fluorescein solution for 10 minutes. Subsequently, the samples were thoroughly washed with distilled water and sonicated in distilled water until there was no additional release of physisorbed fluorescein from the samples in order to remove excess physisorbed fluorescein from the samples. The samples were then air dried. The sample treated with ODMcat appeared markedly orange, whereas the control sample (untreated filter paper) appeared white (FIG. 1). The orange color is due to the retained fluorescein dye molecules bound to covalently attached quaternary amino groups from the ODMcat moieties.

### Example 5: Grafting on Cotton

### Depositing and Grafting

Cotton was soaked in an alcoholic solution of ODMcat. Cotton samples were baked in an oven at 110° C. for one hour. Control cotton was soaked in distilled water. The samples were then sonicated in ethanol for 10 minutes to remove any excess of reagent. The samples were then sonicated for 10 minutes in distilled water, and then rinsed with distilled water.

### Visual Fluorescein Test

Samples were treated according to the visual fluorescein test described in Example 4. FIG. 2 shows images of control and ODMcat-treated cotton after vortexing, sonication and drying. The orange appearance of ODMcat-treated cotton is due to the extremely high number of counter ion of the fluorescein dye bound to the quaternary amino groups of the ODMcat moiety, which is covalently attached to the cotton.

### Example 6: Grafting on Glass

### Glass Preparation and Activation

4.84 cm² glass coverslips (22 mm×22 mm) were sonicated in acetone, ethanol, and distilled water. The coverslips were then placed in a piranha solution [96% sulfuric acid/30% hydrogen peroxide (2/1, v/v)] for 10 minutes to create Si—OH group on the glass surface. All coverslips were subsequently rinsed with distilled water, sonicated in distilled water and air dried.

### Depositing and Grafting

The solution prepared according to Example 3 was deposited on the activated glass coverslips by spin-coating, and then covalently grafted on the activated glass (22 mm×22 mm coverslips). After deposition, samples were transferred to an oven at 110° C. for 60 minutes. Samples were then retrieved and sonicated in ethanol for 5 minutes for adequate removal of any remaining physisorbed reagent. Samples were then sonicated for 5 minutes in distilled water, and rinsed with distilled water.

### Surface Charge Determination by Fluorescein Test

Surface cationic density (N⁺/cm²) was calculated using UV/Vis absorbance of fluorescein. Fluorescein dye molecules (negatively charged) strongly bind to quaternary amino groups (positively charged) belonging to the polymeric chain. The stoichiometry of fluorescein binding is estimated at one dye molecule per four quaternary ammonium moieties. Treated plates were placed in a 1% fluorescein aqueous solution for five minutes, and then rinsed and sonicated in distilled water for several minutes in order to remove physisorbed fluorescein. Plates were rinsed with distilled water and immersed in 3 mL of CTAB 0.5% (aqueous solution of cetyltrimehylammonium bromide) along with PBS (Phosphate-Buffered Saline) (90% CTAB/10% PBS) and sonicated for 10 minutes. The optical density of chemisorbed fluorescein was measured at 501 nm wavelength.

Surface cationic density was estimated according to the following formula:

$$\frac{A \times V \times NA}{ε \times S}$$

A: absorbance of fluorescein solution at 501 nm
V: volume (3 mL)
N_{A}: Avogadro's number (6.022140857×10²³ mol⁻¹)
ε: fluorescein molar absorptivity or molar extinction coefficient (L×mol⁻¹×cm⁻¹).
S: surface in cm²

Measurements were as follows:
Spin-coated treated glass slides (150 μL deposit, 4000 RPM, 40 seconds, 3 mL CTAB/PBS)

$$A = 0.021 \pm 0.003$$

Calculated surface cationic density: 4×10¹⁴ cations/cm²

These results confirmed the effective grafting of the catechol-based quaternary ammonium moiety on glass.

### Example 7: One-Pot Synthesis of Graftable Biocidal Polymers

### General Synthesis for Polymer Based on Poly(4-Vinylpyridine)

Poly(4-vinylpyridine) (PVP; Mw 60,000 g/mol; 1 eq.) was reacted with 4-(chloroacetyl)catechol (also known as 2-chloro-3,4-dihydroxyacetophenone) or 4-(iodoacetyl)catechol (0.1 to 0.5 eq) and an alkyl chain (for example, a C4 to C12 carbon chain) in an alcoholic solvent to prepare a random copolymer having biocidal properties. The solution was refluxed overnight. Subsequently, a C4 to C12 alkyl-halide (excess) was added (to check the best length for optimal biocidal activity. The reaction was stopped anytime between 24 hours and 4 days depending on the desired N+/N quaternization ratio. Partial quaternization resulted in a polymer displaying both biocompatibility and biocidal activity. Maximum yield of quaternarization results in a polymer displaying high biocidal activity. As depicted in Scheme 15, 1 equivalent of PVP is used to prepare a random copolymer that comprises a molar ratio of A to B of x:(1-x), wherein 0.1≤x≤0.5.

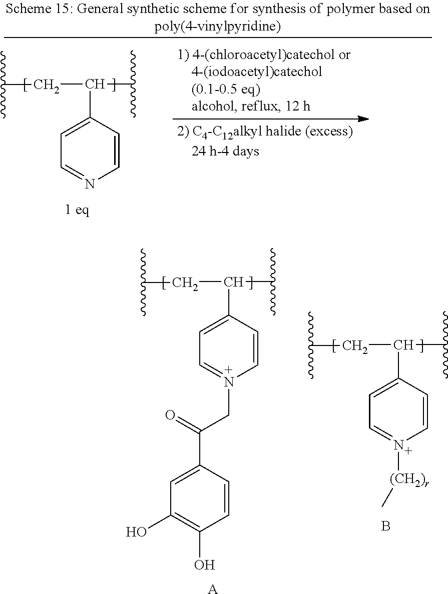

### Example 8: Synthesis of Random Copolymer (C1)

In a one-pot process, a random copolymer was prepared by reacting iodopropyltrimethoxysilane (0.05 eq.), polyvinylpyridine (PVP) (1 eq.) and bromobutane (1 eq.) in boiling isopropanol for a reaction time of four days.

### Example 9: Synthesis of a Novel Random Copolymer: Catecholacetyl-Cobutyl PVP (C2)

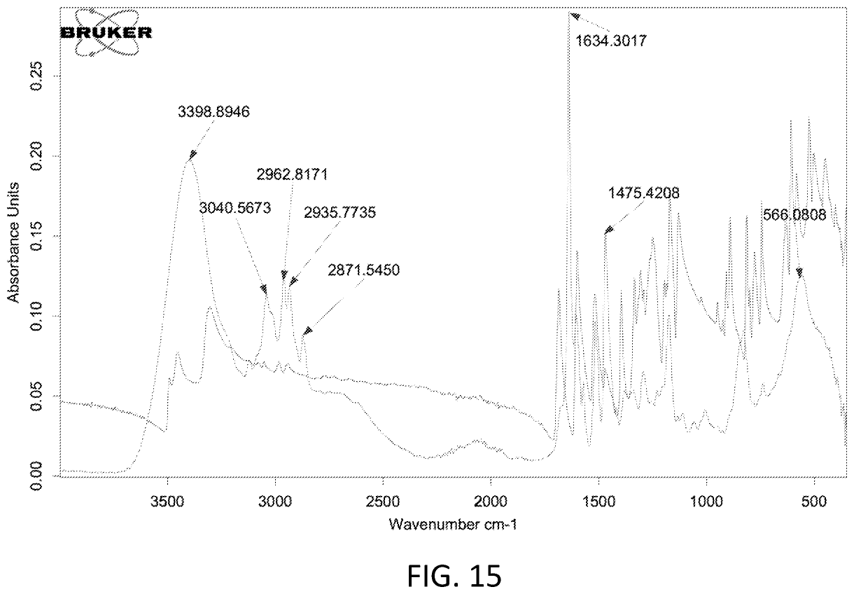

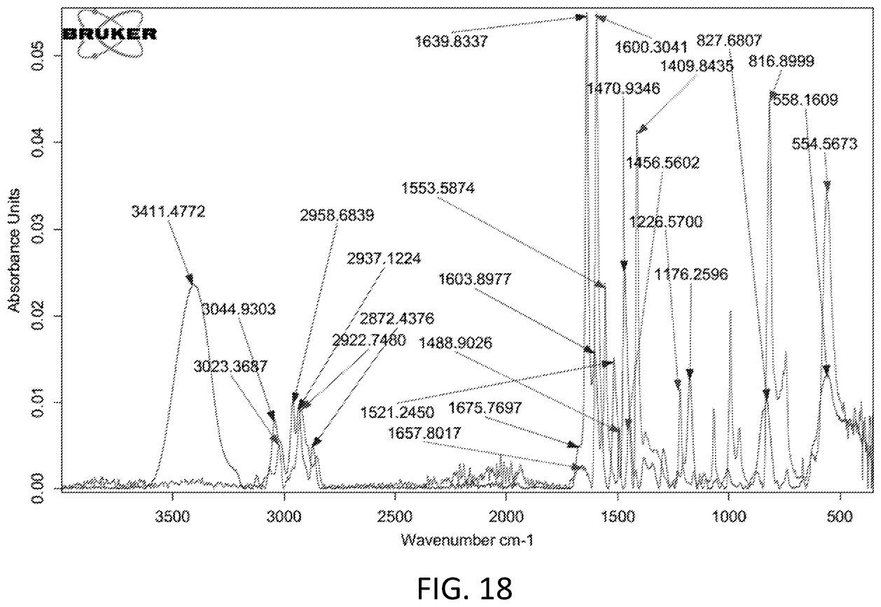

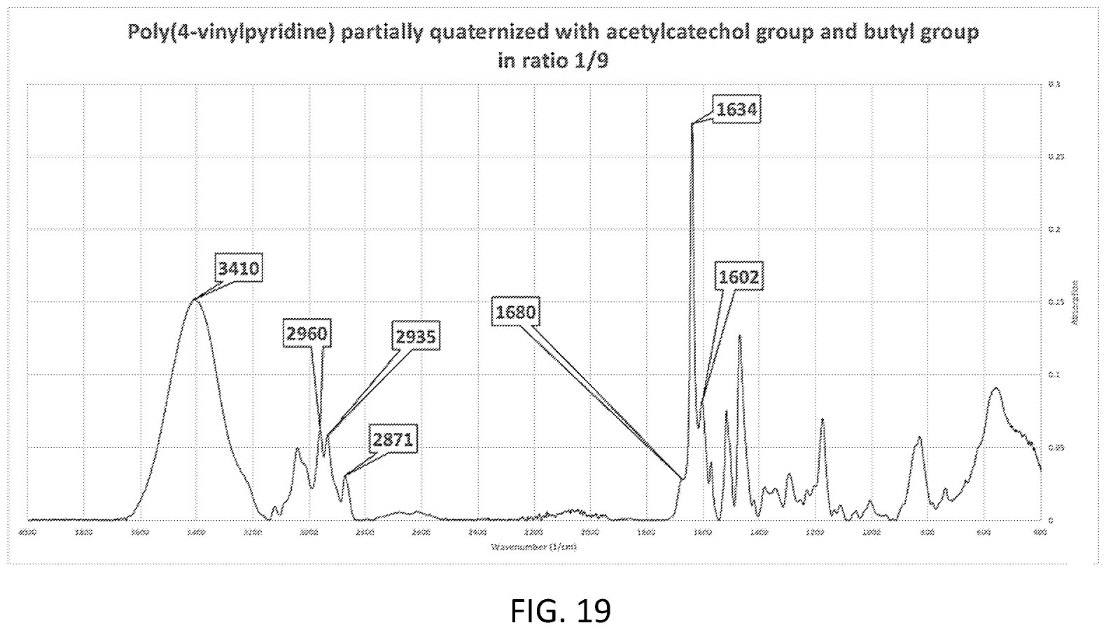

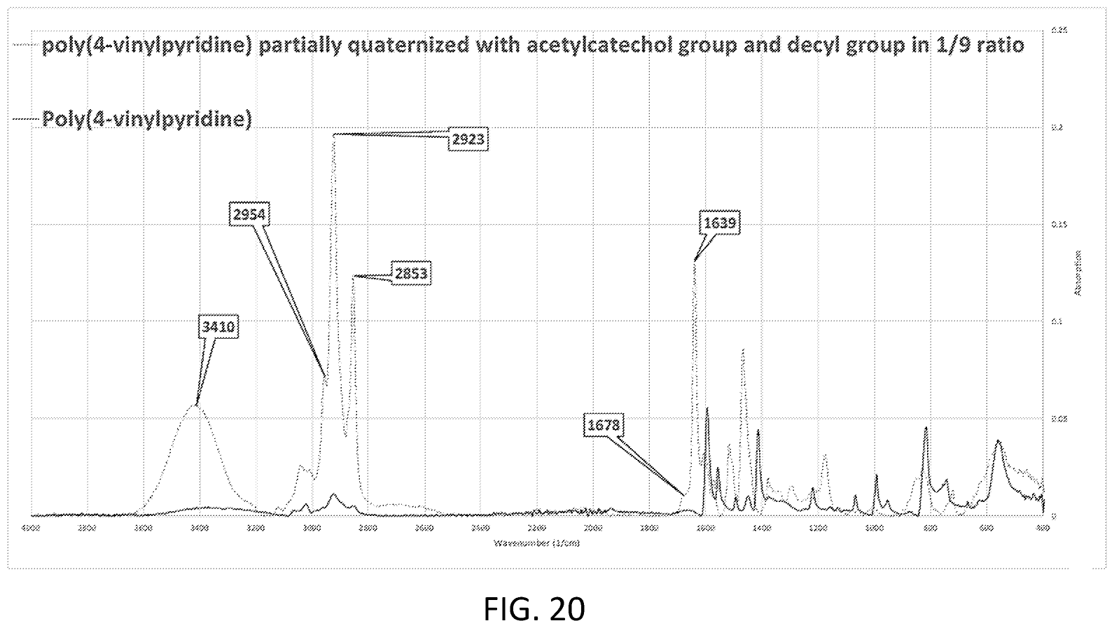

In a one-pot process, a solution comprising a random copolymer was prepared by reacting 4-chloroacetylcatechol (0.06 eq.), polyvinylpyridine (PVP) (1 eq.) and bromobutane (1 eq.) in boiling isopropanol for a reaction time of four days. Scheme 16 depicts the general synthetic scheme. The molar ratio of A to B in the random copolymer is 0.06:0.94. The IR spectrum of catecholacetyl-cobutyl PVP is shown in FIG. 15. FIG. 18 illustrates an IR spectrum of the catecholacetyl-cobutyl PVP co-polymer (C2) with non-quaternized PVP in the background. FIG. 19 illustrates an IR spectrum of the catecholacetyl-cobutyl PVP co-polymer (C2). The PVP-catechol-codecyl-PVP co-polymer was also prepared using similar methods, demonstrating successful modification of the lateral alkyl chain. FIG. 20 illustrates an IR spectrum of the catecholacetyl-codecyl PVP co-polymer with commercial non-quaternized PVP in the background.

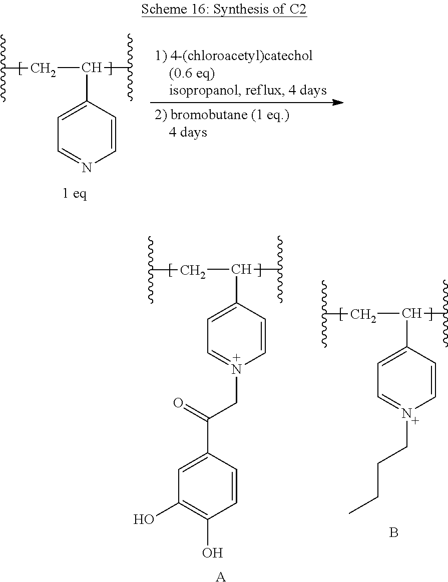

### Example 10: Polymer Grafting on Titanium Surfaces

### Plate Preparation and Activation

1 cm² titanium plates (99.6% purity, Goodfellow, Cambridge Ltd., Huntington, United Kingdom) were successively polished on 3 grains (P800, P2000 and P4000 grit paper) for 1 minute, 2 minutes, and 3 minutes, respectively. After exhaustive washings and a 5-minute sonication process with distilled water in order to remove residual particles, the plates were placed in a piranha solution [96% sulfuric acid/30% hydrogen peroxide (2/1, v/v)] for two minutes to activate the surfaces. All plates were subsequently rinsed, sonicated in distilled water and air dried.

### Deposition and Grafting

The solution of random copolymers prepared according to Example 9 (C2) was deposited by either by spin-coating or dip coating on activated plates. The plates were then baked overnight in an oven at 110° C. The plates were then sonicated in ethanol for 10 minutes to remove any excess of reagent. Plates were then sonicated for 10 minutes in distilled water, and rinsed with distilled water.

### Surface Charge Determination by Fluorescein Test

Surface cationic density (N⁺/cm²) was calculated by a fluorescein test as described above in Example 6. According to Kügler et al., the charge-density threshold for optimum efficiency of biocidal cationic surfaces is ≈10¹⁵ cations/cm².

The following measurements were made:

Titanium plates (1 cm²) were spin-coated with an aqueous solution of 5% C2 and baked in an oven at 110° C. overnight (150 μL deposit, 4000 RPM, 40 seconds). The surface cation density calculation was based on the use of 3 mL CTAB/PBS solution:

$$A = 0.079 \pm 0.005$$

Calculated surface cationic density: 7.44×10¹⁵ cations/cm²

Titanium plates (1 cm²) were dip-coated with an aqueous solution of 5% C2 and baked in an oven at 110° C. overnight (150 μL deposit, 4000 RPM, 40 seconds). Calculation was based on the use of 3 mL CTAB/PBS solution.

$$A = 0.139 \pm 0.009$$

Calculated surface cationic density: 1.31×10¹⁶ cations/cm²

Whether C2 was spin-coated or dip-coated on titanium, it systematically displayed a high cationic charge density above the threshold for optimum efficiency (biocidal activity).

### Example 11: Grafting on Cotton

### Deposition and Grafting

A solution of random copolymer as prepared according to Example 9 (C2) was used to soak cotton for 5 minutes. Control cotton was soaked in distilled water. Treated and control cotton were baked in an oven at 110° C. for 60 minutes. Samples were then sonicated in ethanol for 10 minutes to remove any excess of reagent. Samples were then sonicated for 10 minutes in distilled water, and rinsed with distilled water.

### Visual Fluorescein Test

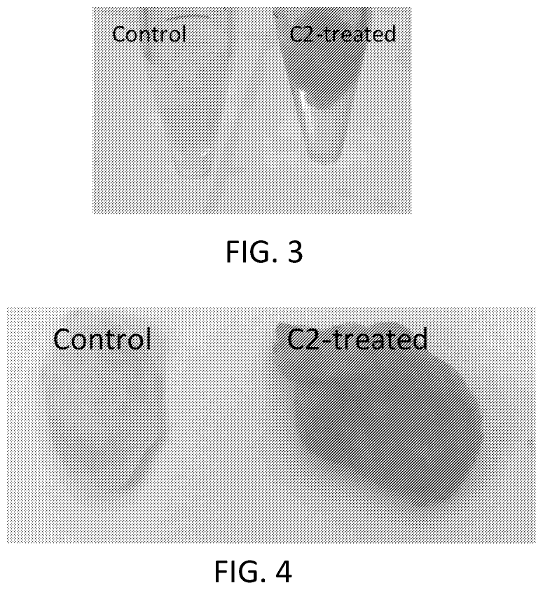

Samples were treated according to the visual fluorescein test described in Example 4. FIG. 3 shows images of the control and C2-treated cotton after vortexing. FIG. 4 shows images of the control and C2-treated cotton after vortexing, sonication and drying. The orange appearance of C2-treated cotton is due to the high number of counterions of the fluorescein dye bound to the quaternary amino groups of the C2 moiety, which is covalently attached to the cotton.

### Example 12: Grafting on Filter Paper

### Deposition and Grafting

A 1 cm² dry filter paper sample was impregnated with 4 drops of aqueous 5% C2 (synthesized according to Example 9) and baked in an oven at 110° C. for 60 minutes. A control filter paper sample was impregnated with distilled water. Samples were then sonicated in ethanol for 10 minutes to remove any excess of reagent. Samples were then sonicated for 10 minutes in distilled water, and rinsed with distilled water.

### Visual Fluorescein Test

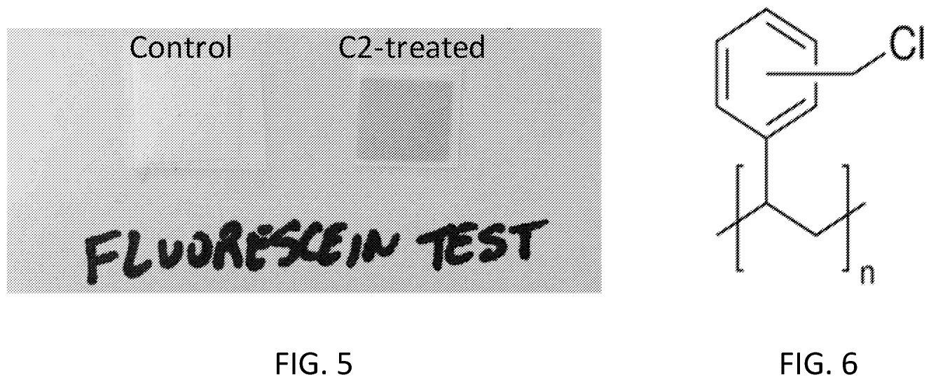

Samples were treated according to the visual fluorescein test described in Example 4. FIG. 5 illustrates images of the control and C2-treated filter paper after fluorescein test. The orange appearance of C2-treated filter paper is due to the high number of fluorescein dye molecules bound to the quaternary amino groups of the C2 moiety, which is covalently attached to the cotton.

### Example 13: Grafting on Aluminum

### Plate Preparation and Activation

4 cm² and 1 cm² aluminum 1060 plates were sonicated in acetone, then in ethanol, and then in distilled water. The plates were immersed in an NaOH 0.1M solution for 20 minutes to remove the protective layer of aluminum oxide to provide Al—OH bonds. All plates were subsequently rinsed, sonicated in distilled water for 10 min, and air dried.

### Deposition and Grafting

Solutions of random copolymers as prepared according to Example 8 (C1) and Example 9 (C2) were deposited by either by spin-coating, dip-coating, or immersion on activated plates. The plates were baked in an oven at 110° C. overnight. Samples were then sonicated in ethanol for 10 minutes to remove any excess of reagent. Samples were then sonicated for 10 minutes in distilled water, and rinsed with distilled water.

### Surface Charge Determination by Fluorescein Test

Surface cationic density (N⁺/cm²) was calculated by a fluorescein test as described in Example 6. According to Kügler et al., the charge-density threshold for optimum efficiency of biocidal cationic surfaces is ≈10¹⁵ cations/cm²

The following measurements were made:

Aluminum plates (4 cm²) were immersed and placed in an aqueous solution of 5% C2 (3 mL) and baked in an oven at 110° C. for 3 hours. Calculation was based on the use of 15 mL CTAB/PBS solution.

$$A = 0.076 \pm 0.007$$

Calculated Surface cationic density: 4.44×10¹⁶ cations/cm²

5% C2 dip-coated aluminum plates (4 cm²) were baked in an oven at 110° C. for 3 hours. Calculation was based on the use of 15 mL CTAB/PBS solution.

$$A = 0.150 \pm 0.015$$

Calculated Surface cationic density: 1.4×10¹⁶ cations/cm²

Aluminum plates (4 cm²) were immersed in a solution of C1 as prepared in Example 8 and baked in an oven at 110° C. for 3 hours. Calculation was based on the use of 3 mL CTAB/PBS solution.

$$A = 0.043 \pm 0.0013$$

Calculated Surface cationic density: 1.04×10¹⁵ cations/cm²

These results demonstrate that C2 has a higher surface cationic density and value of A than C1, and thus exhibits improved properties over C1.

Due to the high affinity of the catechol group with metals, using these catechol linker provides a very efficient moiety for grafting surface metals such as titanium, aluminum, stainless steel, etc.

### Example 14: Grafting of Glass

### Glass Preparation and Activation

Glass slides were prepared according to Example 6.

### Deposition and Grafting

A solution of random copolymer was prepared according to Example 9 (C2) was deposited by immersion on activated glass. After deposition, samples were baked in an oven at 110° C. for 60 minutes. Samples were then sonicated in ethanol for 10 minutes to remove any excess of reagent. Samples were then sonicated for 10 minutes in distilled water, and rinsed with distilled water.

### Surface Charge Determination by Fluorescein Test

Surface cationic density (N⁺/cm²) was calculated by a fluorescein test as described in Example 6.

The following measurements were made:

Glass slides (10.5 cm²) were immersed in an aqueous solution of 5% C2 as prepared in Example 9 and baked in an oven at 110° C. for 60 minutes. Calculation was based on the use of 3 mL CTAB/PBS solution.

$$A = 0.866 \pm 0.024$$

Calculated Surface cationic density: 7.6×10¹⁵ cations/cm²

### Example 15: Grafting on Balsa Wood

### Sample Preparation

1 cm² squares of balsa wood (1.5 mm thick) were sonicated in acetone, ethanol and distilled water. All squares were then air dried.

### Deposition and Grafting

The samples were placed in a mixture of 5% C2 solution (as prepared in Example 9) in ethanol/water v/v (4 mL) for 30 min. After deposition, samples were baked in an oven at 110° C. for 60 minutes. Samples were then sonicated in ethanol for 10 minutes to remove any excess of reagent. Samples were then sonicated for 10 minutes in distilled water, and rinsed with distilled water.

### Surface Charge Determination by Fluorescein Test

Volumetric cationic concentration (N⁺/cm³) was calculated by a fluorescein test as described above.

The following measurements were made:

Balsa wood samples were immersed in a mixture of 5% C2 solution (prepared as described in Example 9) in ethanol/water v/v (4 mL) and baked in an oven at 110° C. for 1 hour. Calculation was based on the use of 10 mL CTAB/PBS solution.

$$A = 0.5 \; 11 \pm 0.016$$

Volumetric cationic concentration: 1.98×10¹⁶ cations in 0.15 cm³, which corresponds to 1.32×10¹⁷ cations/cm³

### Example 16: Grafting on Stainless Steel

### Stainless Steel Preparation and Activation

4 cm² stainless steel plates were sonicated in acetone, then in ethanol, and then in distilled water. They were then placed in a sulfochromic acid solution for 20 minutes at 50° C. to activate the plate surfaces. All stainless steel plates were subsequently rinsed, sonicated in distilled water for 10 minutes, and air dried.

### Deposition and Grafting

A solution of random copolymer prepared according to Example 9 (C2) was deposited by immersion on activated stainless steel plates. The plates were baked in an oven at 110° C. overnight. Samples were then sonicated in ethanol for 10 minutes to remove any excess of reagent. Samples were then sonicated for 10 minutes in distilled water, and rinsed with distilled water.

### Surface Charge Determination by Fluorescein Test

Surface cationic density (N⁺/cm²) was calculated by a fluorescein test as described in Example 6.

The following measurements were made:

Stainless steel samples (4 cm²) were immersed in an aqueous solution of 5% C2 (prepared as described in Example 9) and baked in an oven at 110° C. overnight. Calculation was based on the use of 3 mL CTAB/PBS solution.

$$A = 0.578 \pm 0.021$$

Calculated Surface cationic density: 6.75×10¹⁵ cations/cm²

### Example 17: General Synthesis for Preparation of a Graftable Polymer from Poly(Vinylbenzyl Chloride)

A graftable polymer is prepared from poly(vinylbenzyl chloride) (FIG. 6). Polyvinylbenzylchloride (MW 55000 g/mol; 1 eq.; mixture of meta and para) is dissolved in THE (tetrahydrofuran). 4-(Dimethylaminoacetyl)catechol is added to the reaction mixture (between 0.05 and 0.20 equivalents). The reaction mixture is heated at reflux for four hours. An excess of a C₄-C₁₂ alkyldimethylamine in a solution of ethanol is added to the reaction mixture, which is heated at reflux for 24 hours, and then cooled down to room temperature. The volume of the reaction mixture is reduced by two-thirds using rotary evaporation, and subsequently cooled down to about 4-8° C. A non-solvent of polycations, such as ether or acetone, is added until the appearance of a precipitate. After cooling the mixture at 4° C. overnight, the precipitate is isolated by vacuum filtration, washed with a non-solvent (ex: ether) and dried under vacuum to provide the desired catechol quaternized polymer. Scheme 17 depicts the general synthesis of the resulting random copolymer that comprises a molar ratio of A to B of x:(1-x), wherein 0.05≤x≤0.5.

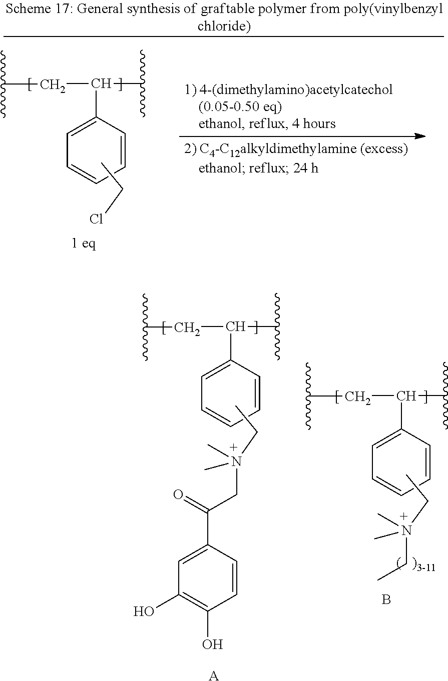

Treating polyvinylbenzylchloride with the dimethylaminoacetylcatechol results in the partial quaternization of polyvinylbenzylchloride. Subsequently, the addition of an excess of the dimethylaminoalkyl chain quaternizes the remaining available sites (unreacted monomer units).

### Example 18: Preparation of a Graftable Polymer from Poly(Vinylbenzyl Chloride)

A graftable polymer is prepared from poly(vinylbenzyl chloride) using the method of Example 17. 4-(Dimethylaminoacetyl)catechol (0.1 eq) and N,N-dimethyldecan-1-amine (excess) are used. The polymer product is a random copolymer (polyvinylbenzyl(N-catecholacetyl-N,N-dialkyl)ammonium bromide) comprising a molar ratio of quaternized nitrogen with a catechol moiety (A in Scheme 17) to quaternized amine with a butyl moiety (B in Scheme 17, where the alkyl chain is 4 carbons) of 0.1:0.9.

### Example 19: General Synthesis for Preparation of a Graftable Polymer from Polyethylenimine (PEI)

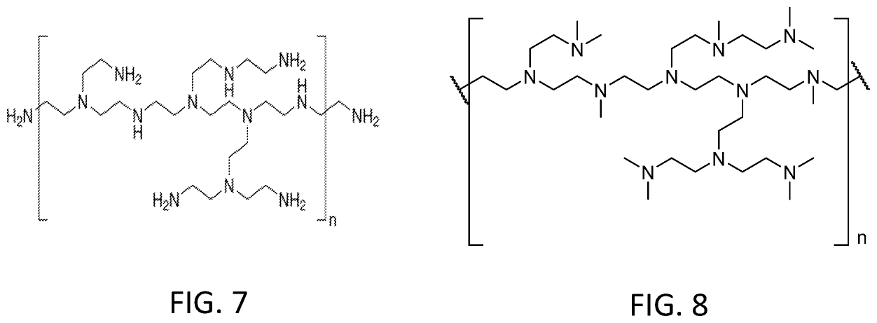

A graftable polymer was prepared from polyethylenimine (PEI) (FIG. 7). PEI contains 53 monomer units, each containing 11 nitrogens (N) that can be quaternized, and can be branched, hyperbranched or linear.

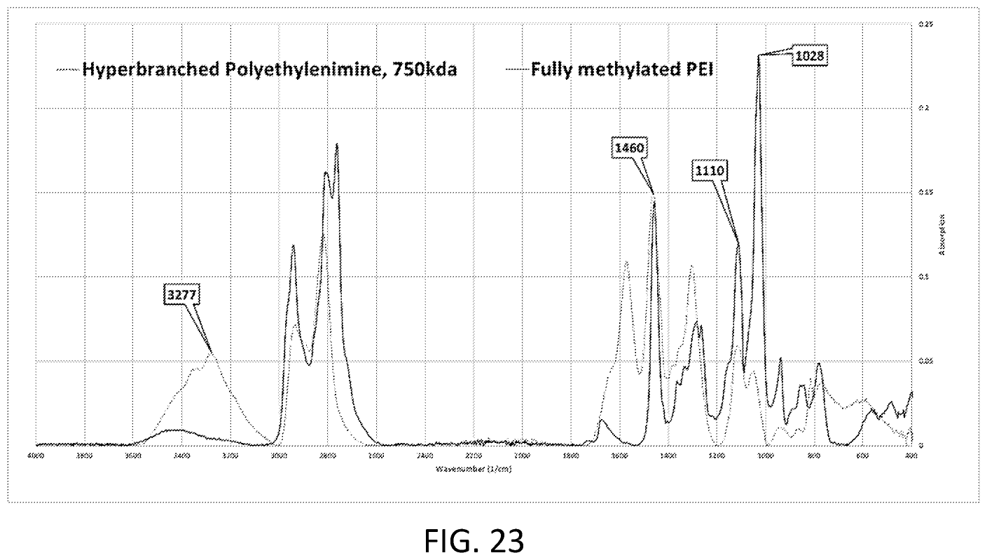

PEI (MW 75,000 g/mol) was treated with a mixture of formaldehyde and formic acid to exhaustively methylate primary and secondary amine groups as depicted below in Scheme 18. It was surprisingly found that PEI with low molecular weight (such as 25,000 g/mol) leads to surfaces exhibiting low cationic charge densities (under 10¹⁵ charges/cm²). FIG. 8 illustrates the structure of PEI after exhaustive methylation. FIG. 23 illustrates IR spectra of methylated hyperbranched PEI (750 kda) and commercial PEI (750 kda).

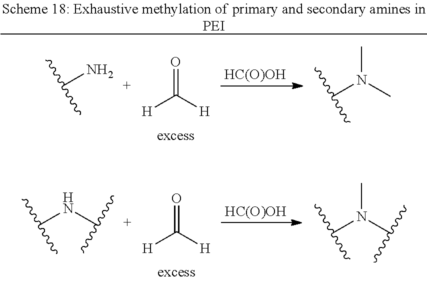

The reaction mixture was neutralized with concentrated aqueous KOH, and then the extracted three times with chloroform. The organic layer was dried over sodium sulphate and evaporated to dryness. The yield was quantitative.

The methylated PEI product was then dissolved in alcohol (such as isopropyl alcohol, t-butyl alcohol, or t-amyl alcohol) and treated with 4-(chloroacetyl)catechol or 4-(iodoacetyl)catechol (0.2-0.5 eq.). The reaction mixture was heated at reflux for several hours. A solution of a C₄-C₁₂ alkyl halide (excess) dissolved in alcohol was added to the reaction mixture, which was then heated at reflux for 2 to 4 days. The reaction mixture was cooled down to about 4-8° C., and the volume of the reaction mixture was reduced to about one third of its initial volume using rotary evaporation. A non-solvent was added until the appearance of a precipitate, and then the reaction mixture was kept at 4° C. overnight. The precipitate was then isolated by vacuum filtration, washed with a non-solvent (ex: ether) and dried under vacuum to provide the desired catechol quaternized PEI polymer. Scheme 19 depicts an example of a fully methylated PEI monomer linked to a catechol moiety.

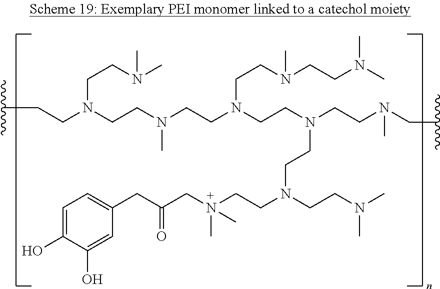

### Example 20: Preparation of a Graftable Polymer from Polyethylenimine (PEI)

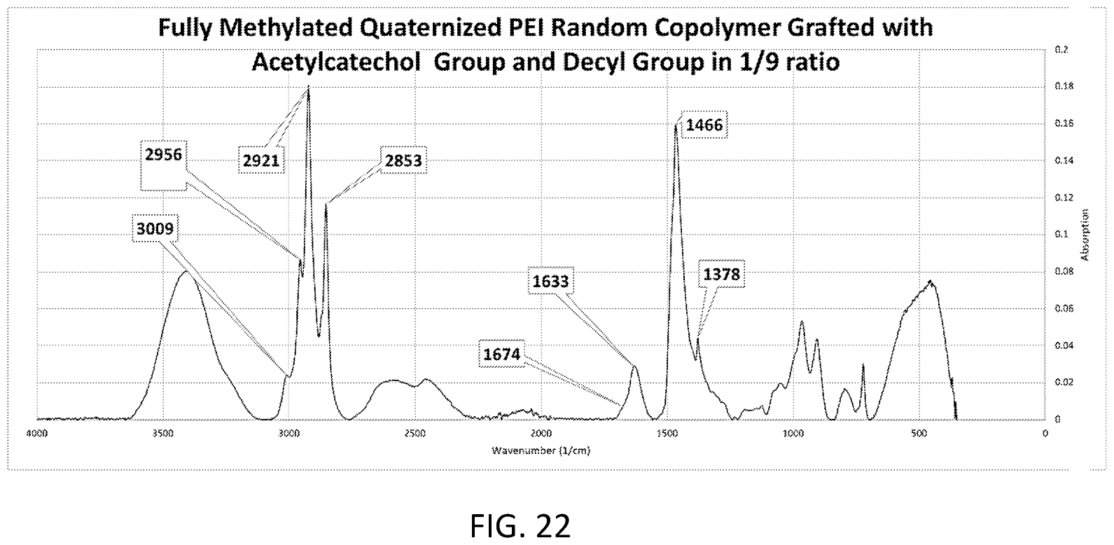

A graftable polymer was prepared from PEI using the method of Example 19. 4-(chloroacetyl)catechol (0.1 eq) and 1-bromodecane (excess) were used. The polymer product was a random PEI copolymer comprising a molar ratio of quaternized nitrogen with a catechol moiety to quaternized nitrogen with a decyl moiety of 0.1:0.9. In some embodiments, 1-bromohexane is used in place of 1-bromodecane. The corresponding PEI polymer with a hexyl moiety instead of a decyl moiety was also prepared. FIG. 22 illustrates an IR spectrum of fully methylated quaternized PEI random copolymer partially grafted with acetylcatechol group and decyl group in ratio 1/9.

### Example 21: General Synthesis for Preparation of a Graftable Polymer from Polyethylenimine (PEI) with an Alkylsilane Moiety

A graftable polymer was prepared from polyethylenimine (PEI) and an alkylsilane moiety, such as 3-iodopropyltrimethoxysilane, using the same procedure described in Example 19, except the alkylsilane (0.05 eq.) is used instead of the 4-(chloroacetyl)catechol or 4-(iodoacetyl)catechol. Scheme 20 depicts an example of a fully methylated PEI monomer linked to a 3-propyltrimethoxysilane moiety.

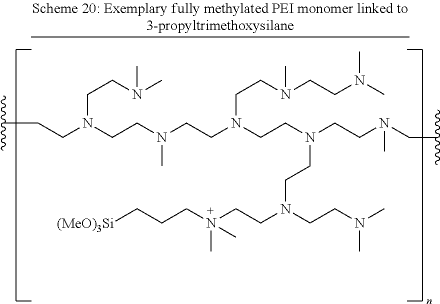

### Example 22: Preparation of a Graftable Polymer from Polyethylenimine (PEI)

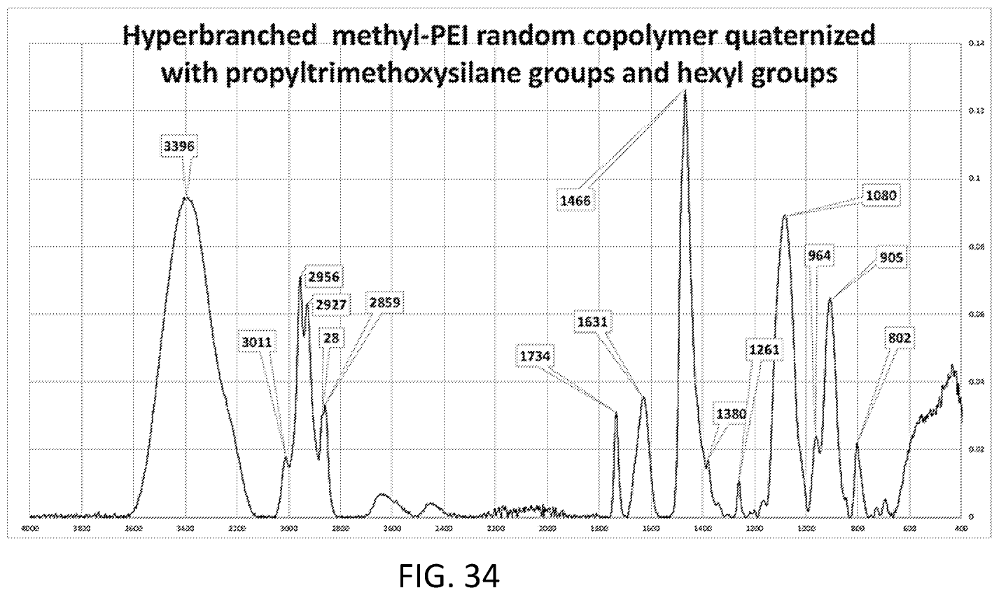

A graftable polymer was prepared from PEI using the method of Example 21. 3-iodopropylmethoxysilane (0.1 eq) was used as the alkylsilane, and 1-bromohexane (excess) was used as the C₄-C₁₂ alkyl halide. The polymer product was a random PEI copolymer comprising a molar ratio of quaternized nitrogen with alkylsilane moiety to quaternized nitrogen with a hexyl group of 0.1:0.9. Partially silanized PEI could not be isolated because of the eventual reticulation of the silane linker in the absence of solvent. In a non-limiting example, the PEI-co-alkyl-PEI is kept in an alcoholic or alcoholic/water solution. FIG. 34 shows an IR spectrum fully methylated quaternized PEI random copolymer partially grafted with propyltrimethoxysilane group and hexyl group in ratio 1/9. The fully methylated quaternized PEI random copolymer partially grafted with propyltrimethoxysilane group and hexyl group in ratio 1/9 was also prepared using similar methods, demonstrating successful modification of the lateral alkyl chain. A fully methylated quaternized PEI random copolymer partially grafted with propyltrimethoxysilane group and decyl group was also prepared.

### Example 23: General Method of Deposition and Grafting of Ready-to-Use Biocidal Polymers

Polymers disclosed herein are grafted onto a variety of hydroxylated surfaces. The surface may be naturally hydroxylated or is artificially activated prior to deposition by methods including oxidant treatment with plasma, acid or UV/ozone. The hydroxylated surface is positioned for deposition. The polymer is deposited onto the surface using methods such as spraying, dip coating, or spin-coating. The polymer is grafted to the surface by heating, for example by heating the surface to 110° C. for 30 to 60 min. The temperature may be adjusted if a different temperature is desired. When grafting at a temperature lower than 110° C., a longer heating period is then utilized. After grafting is complete, the substrate is washed (with an alcohol such as isopropanol) to eliminate physisorbed polymers and retain only covalently grafted polymers. Optionally, the substrate may be sonicated during washing. After washing, the substrate is dried under air atmosphere.

### Example 24: General Preparation of Dipodal Silane Compounds

Example 24 describes the preparation of novel dipodal silane compounds and moieties, which can be covalently grafted onto a variety of surfaces. Various dipodal silane compounds are further substituted with an alkyl chain by treatment with an alkyl halide in a single step reaction to provide highly hydrophobic/hydrophilic compounds that are ready-to graft on a variety of surfaces, including hydroxylated or activated surfaces, with the benefit of being much more stable and extremely resistant to hydrolysis compared to conventional silanes. In an non-limiting example, a dipodal silyl amine compound is treated with an alkyl halide, such as a C₄-C₂₂ alkyl halide, in an alcohol such as isopropanol, and heated to reflux for 24-96 hours.

### Example 25: Preparation of bis[3-(trimethoxysilyl)propyl-N,N′-tetraalkylethylenediammonium compounds

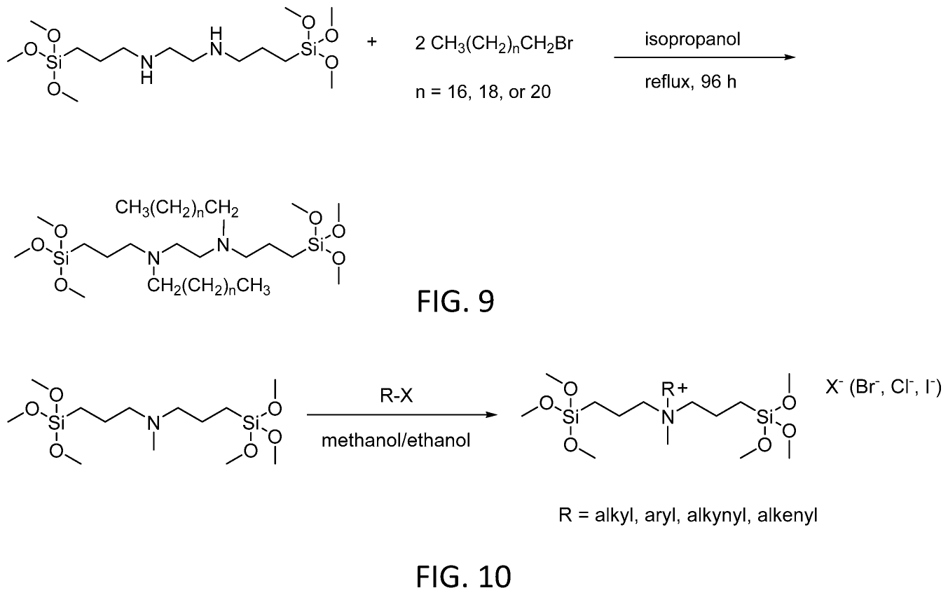

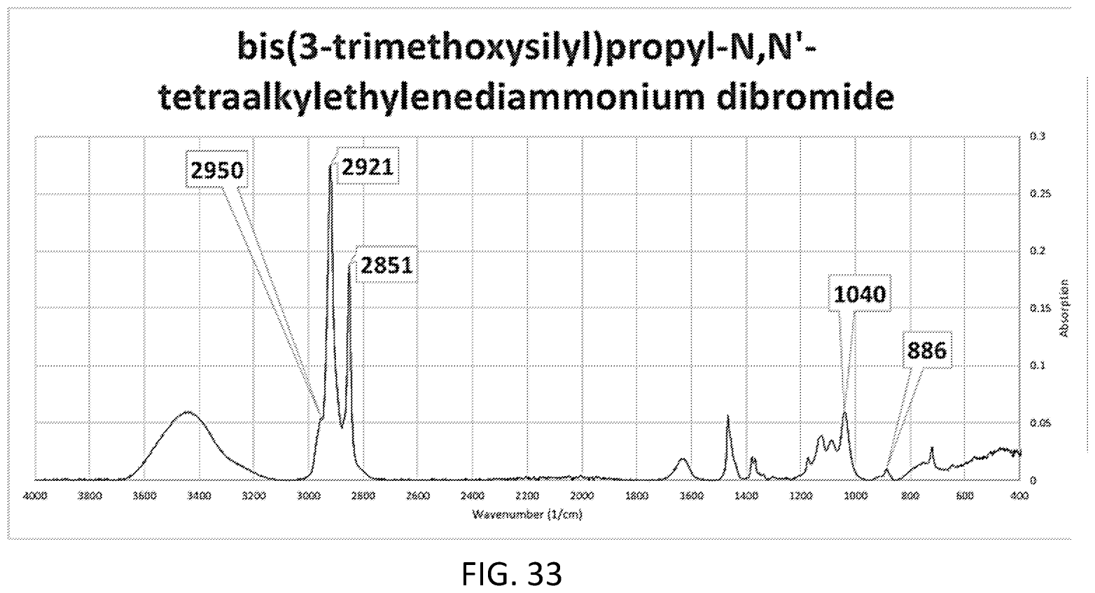

N,N′-Bis[3-(trimethoxysilyl)propyl]ethylenediamine (1 eq.) is treated with an alkyl halide (2 eq.), such as a C₁₈-C₂₂ alkyl bromide, in isopropanol and heated at reflux for 96 h to produce bis[3-(trimethoxysilyl)propyl-N,N′-tetraalkylethylenediammonium compounds. An exemplary reaction scheme is depicted in FIG. 9. The spectrum is provided in FIG. 33.

### Example 26: General Preparation of Quaternized Dipodal Silane Compounds

A compound prepared by Example 24 is quaternized by treatment with at least 1 equivalent of an alkyl halide, such as a C₄-C₂₂ alkyl halide, in an alcohol, such as isopropanol, heated to reflux for 24-96 h. These quaternary ammonium compound exhibit antimicrobial properties, and provide same advantage of using dipodal silanes as described in Example 24.

### Example 27: Preparation of bis(3-trimethoxysilylpropyl)-N,N-methylalkyllammonium bromide

Bis(3-trimethoxysilylpropyl)-N-methylamine (1 eq.) is treated with an alkyl halide (1 eq.), such as a C₁₈-C₂₂ alkyl bromide, in isopropanol and heated at reflux for 48 h to produce bis(3-trimethoxysilylpropyl)-N,N-methylalkyllammonium bromide. An exemplary reaction scheme is depicted in FIG. 10. The advantage of grafting such a dipodal quaternary ammonium compound is to confer superior robustness to the grafted film thanks to an improved reticulation within the film. The resulting compound resists hydrolysis significantly better than monopodal-silane quaternary ammonium compounds.

### Example 28: Preparation of Quaternized Bis(3-Trimethoxysilylpropyl)Dialkylammonium Bromide

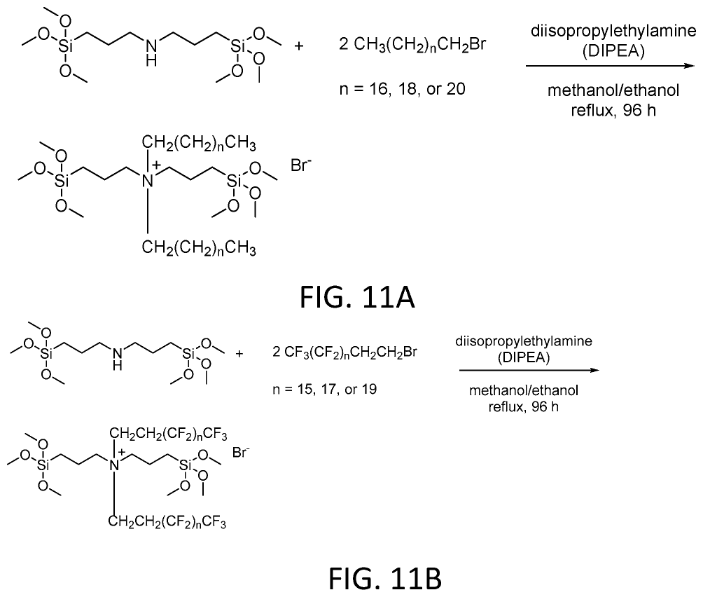

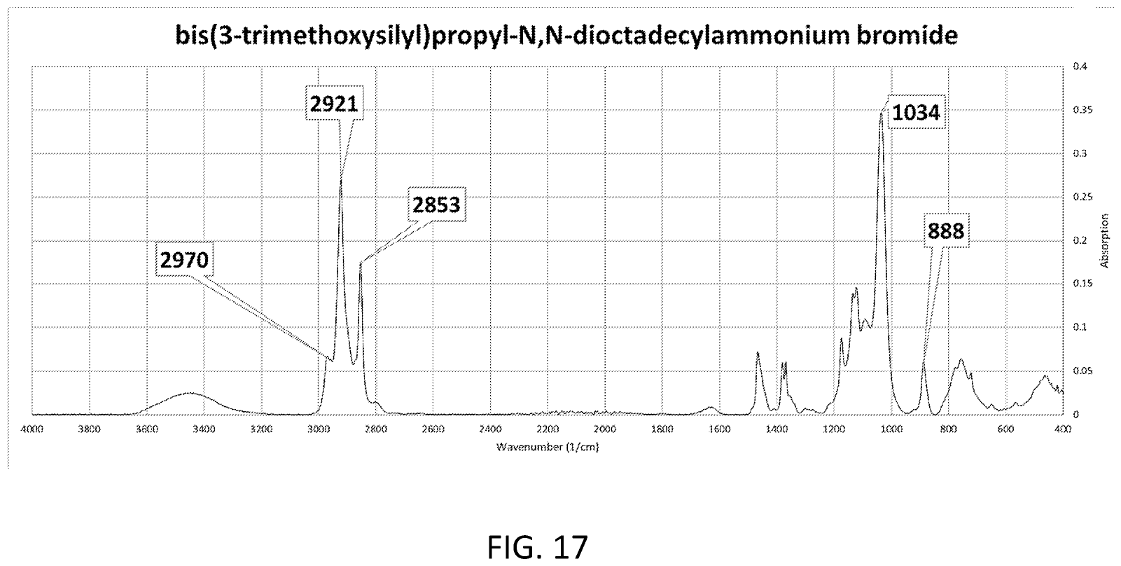

Bis(3-trimethoxysilylpropyl)amine (1 eq.) is treated with an alkyl halide (2 eq.), such as a C₁₈-C₂₂ alkyl bromide, in isopropanol and heated at reflux for 96 h to produce bis(3-trimethoxysilylpropyl)-N,N-dialkyllammonium bromide. An exemplary reaction scheme is depicted in FIG. 11A and FIG. 11B. FIG. 17 illustrates an IR spectrum of bis(3-trimethoxysilyl)propyl-N,N-dioctadecyl ammonium bromide. Bis(3-trimethoxysilyl)propyl-N,N-dihexadecyl ammonium bromide can also be prepared using similar methods using bromohexadecane instead of bromooctadecane.

### Example 29: Preparation of a Random Dipodal Polyvinylpyridine (PVP) Copolymer

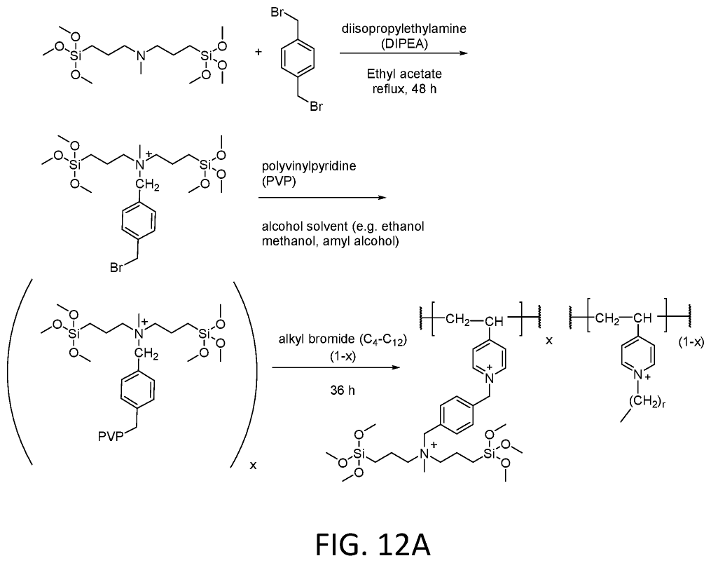

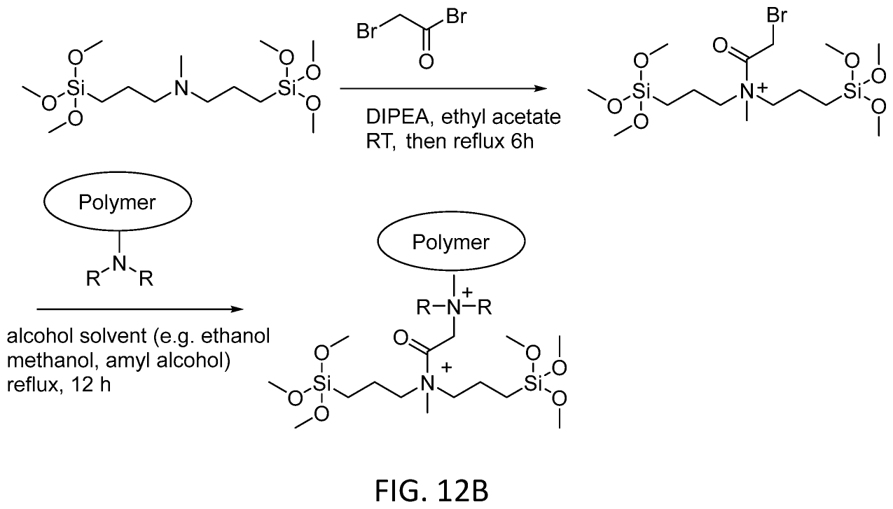

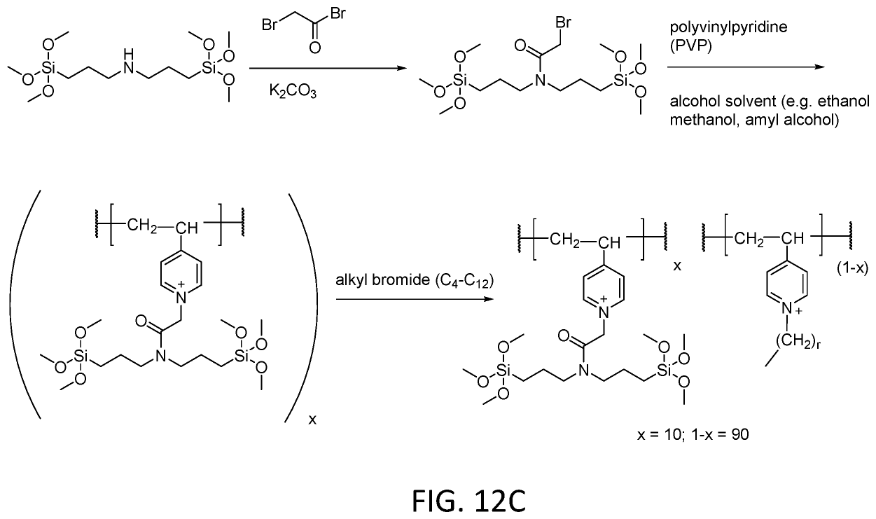

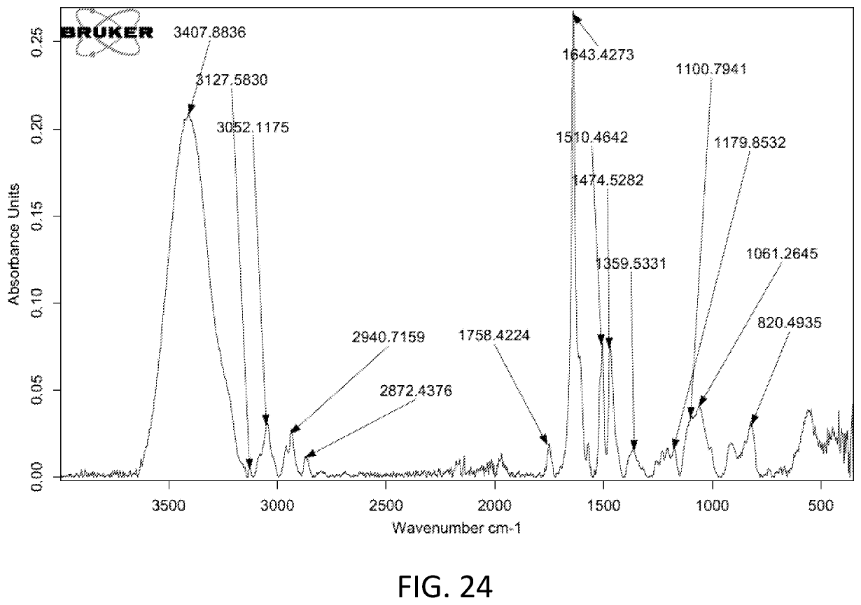

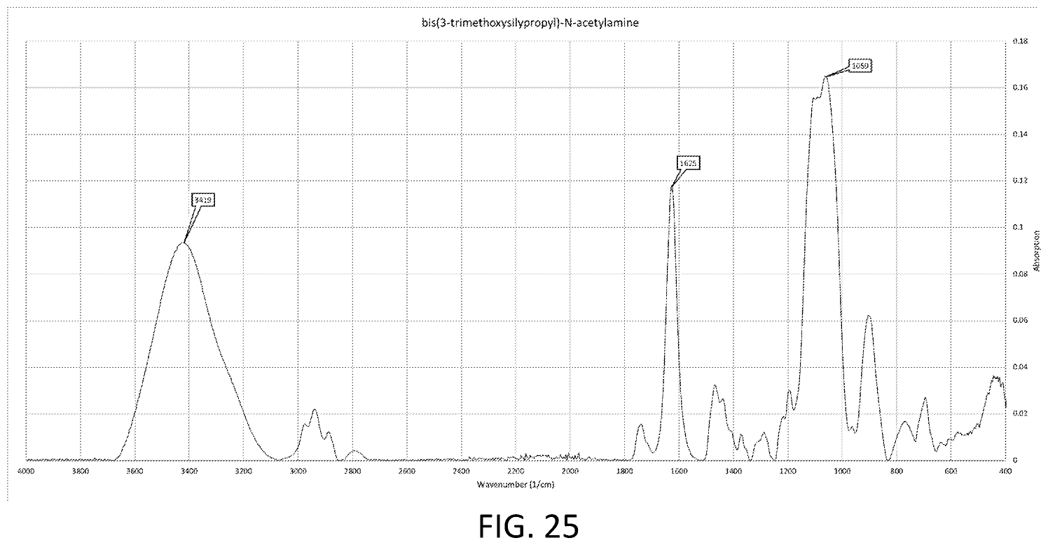

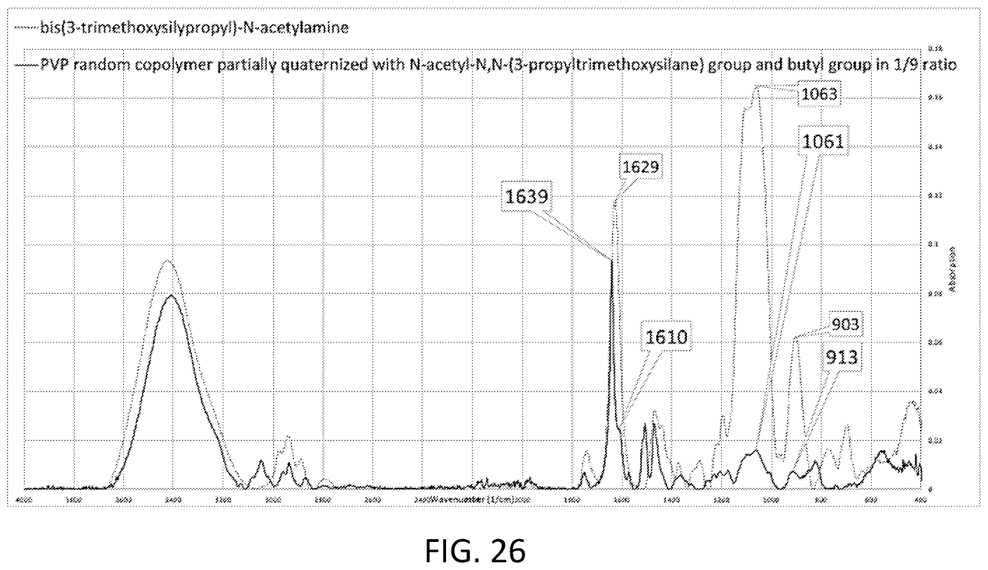

Bis(3-trimethoxysilylpropyl)-N-methylamine was treated with 1,4-bis(bromomethyl)benzene (0.05 eq. to 0.2 eq., for example 0.05 eq.) in ethyl acetate stirred at reflux for 48 h to produce a quaternary amine compound, which was then treated with PVP in isopropanol and stirred at reflux for 12 h. FIG. 25 shows an IR spectrum of the quaternary amine compound named bis(3-trimethoxysilypropyl)-N-bromoacetylamine, which is a linker for PVP. The polymer was then treated with at least 1 equivalent of an alkyl halide, such as a C₄-C₂₂ alkyl halide, in an alcohol (for example, isopropanol), to provide a random PVP copolymer substituted with a molar ratio of the bis(3-trimethoxysilylpropyl)-N-methyl-N-paraxylyl moiety to the alkyl group moiety of about (0.05≤x≤0.2)(1-x). FIG. 26 shows an IR spectrum of the dipodal quaternized PVP with a C4 lateral chain (red line) compared to the intermediate bis(3-trimethoxysilypropyl)-N-bromoacetylamine (blue line). In some embodiments, when 0.05 eq of bis(3-trimethoxysilypropyl)-N-bromoacetylamine is used, the molar ratio is about 0.05:0.95. FIG. 12A and FIG. 12B illustrate exemplary reaction schemes for linking a bis(3-trimethoxysilylpropyl)-N-methyl-N-paraxylyl moiety to a polymer comprising a monomer comprising a tertiary amine to form a quaternary amine. Non-limiting examples of polymers that comprise tertiary amines include polyvinylpyridine (PVP and polyethylenimine (PEI). The quaternized bis(3-trimethoxysilylpropyl)-N-methyl-N-paraxylyl-codecyl-PVP co-polymer was also prepared using similar methods but replacing bromobutane by bromodecane, demonstrating successful modification of the lateral alkyl chain. FIG. 24 illustrates an IR spectrum of dipodal quaternized PVP with a C4 lateral chain (quaternized bis(3-trimethoxysilylpropyl)-N-methyl-N-paraxylyl-cobutyl-PVP).

### Example 30: Preparation of a Random Poly(Vinylbenzyl Chloride) Copolymer

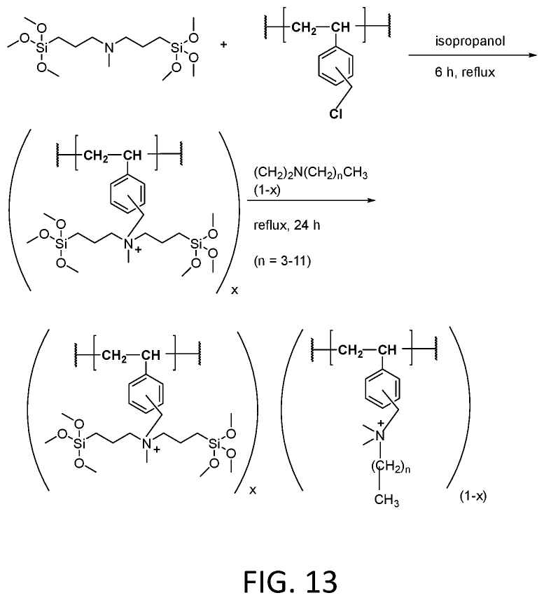

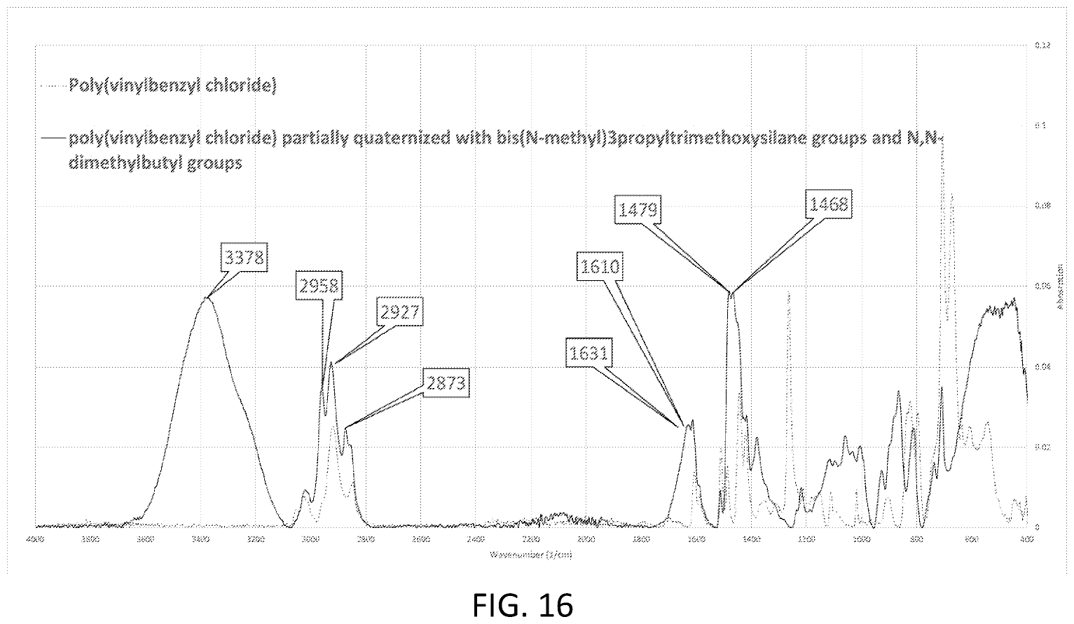

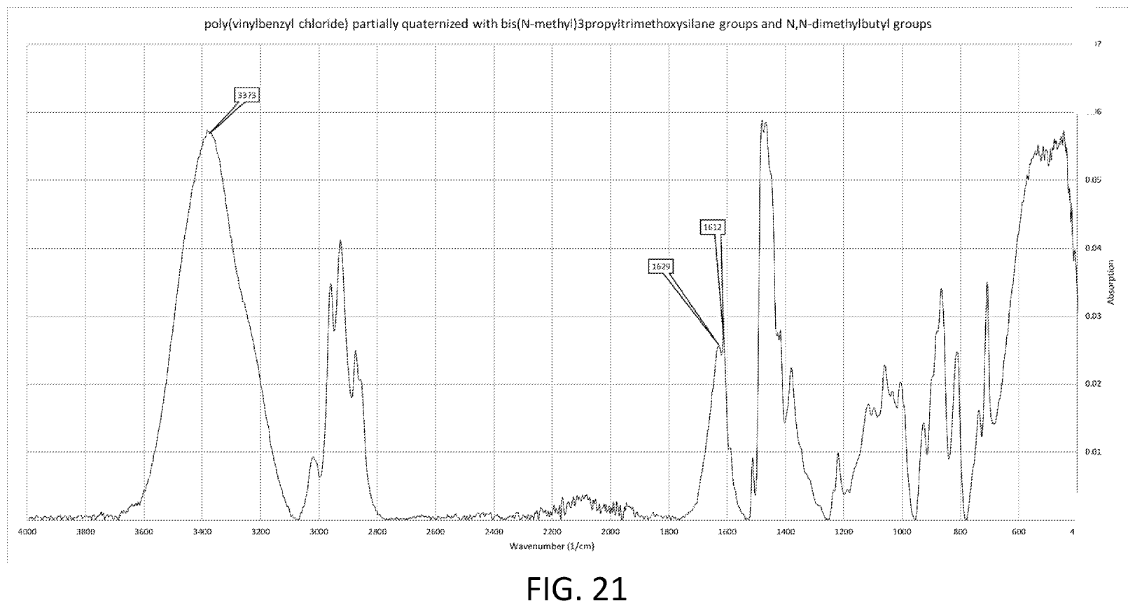

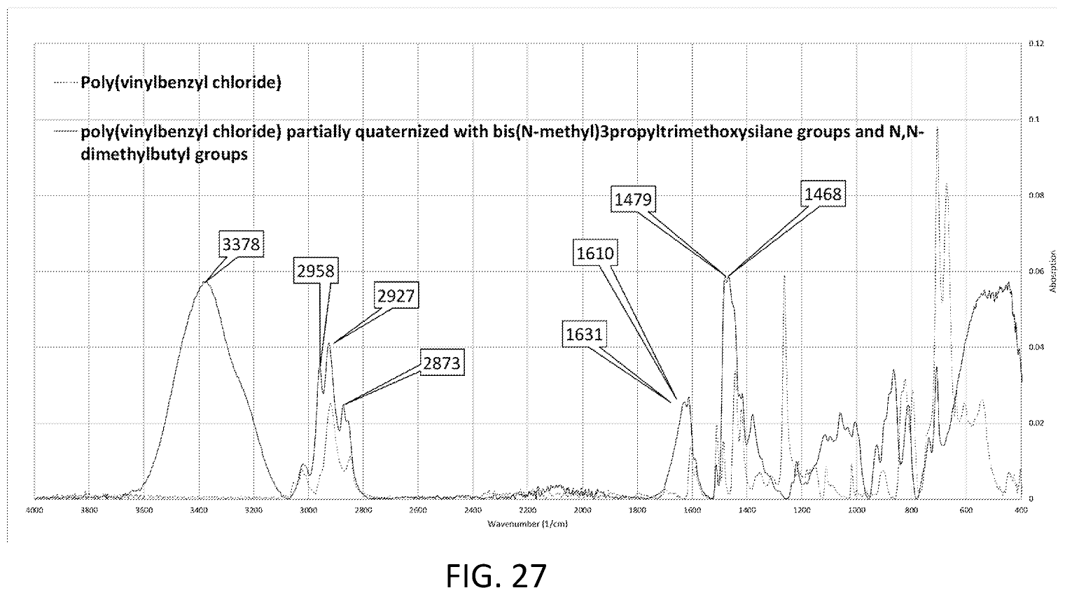

Bis(3-trimethoxysilylpropyl)-N-methylamine (0.05 eq. to 0.2 eq., for example 0.05 eq.) was treated with poly(vinylbenzyl chloride) (1 eq.) in isopropanol and stirred at reflux for 6 h. The polymer was then treated with a treatment with at least 1 equivalent of a C₄-C₁₂ alkyldimethylamine in an alcohol (for example, isopropanol) and stirred at reflux for 24 h to provide a random poly(vinylbenzyl chloride) copolymer substituted with a molar ratio of the bis(3-trimethoxysilylpropyl)-N-methylamine moiety to the C₄-C₁₂ alkyldimethylamine moiety was about (0.05≤x≤0.2)(1-x). In some embodiments, when 0.05 eq of bis(3-trimethoxysilylpropyl)-N-methylamine is used, the molar ratio is about 0.05:0.95. An exemplary reaction scheme is depicted in FIG. 13. The IR spectrum of the PVBC copolymer is shown in FIG. 16. FIG. 27 shows an IR spectrum of the dipodal quaternized PVBC polymer with a C₁₀ lateral chain (blue line) compared to bis(3-trimethoxysilypropyl)-N-bromoacetylamine (black line). FIG. 21 illustrates an IR spectrum of the poly(vinylbenzyl chloride) co-polymer partially quaternized with bis(N-methyl)3propyltrimethoxysilane groups and N,N-dimethylbutyl groups.

### Example 31: Electrostatic Spraying of a Biocidal Polymer Coupled with Titanium Anatase

The preparation of catecholacetyl-coalkyl PVP (Scheme 21) was described in U.S. Pat. No. 10,743,539, which is incorporated by reference herein in its entirety.

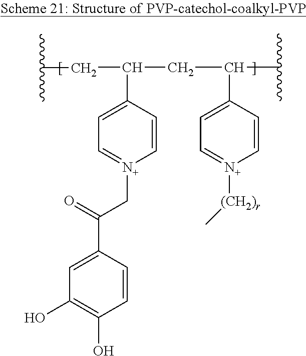

The preparation of octadecyl(4-catecholacetyl)dimethylammonium chloride is described in Example 3 above (Scheme 22)

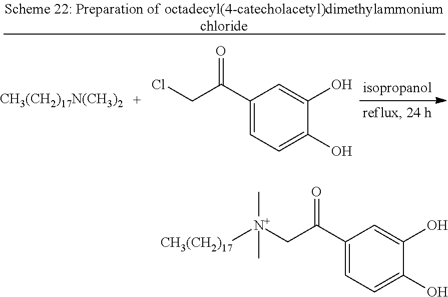

An ethanolic solution containing 1% random copolymer catecholacetyl-coalkyl-PVP bearing catechol groups and alkyl groups with a 1:10 ratio and 1% weight/weight of octadecyl(4-catecholacetyl)dimethylammonium chloride is prepared. In this Example, the mixture of these two compounds (catecholacetyl-coalkyl-PVP and octadecyl(4-catecholacetyl)dimethylammonium chloride) is referred to as biocidal polymer coating solution.

A 0.8% titanium anatase phase in aqueous solution (an equal mixture of peroxotitanium acid and peroxo-modified anatase sol) was prepared according to Ichinose et al., Journal of Sol-Gel Science and Technology 22:33-40 (2001), which is incorporated by reference herein in its entirety.

A glass slide was treated according to the following protocol:
1) The surface was prepared by cleaning with isopropanol, and allowed to dry;
2) The sprayer was held about 1 m from the target surface.
3) The titanium anatase phase in aqueous solution (a mixture of peroxotitanic acid and peroxo-modified anatase sol 0.8%) was sprayed onto the surface using an electrostatic sprayer to form a coating on the target surface.
4) The surface was allowed to dry for 5 to 10 minutes.
5) The electrostatic sprayer was rinsed with distilled water.
6) The biocidal polymer coating solution was added to the sprayer tank.
7) The biocidal polymer coating solution was sprayed onto the surface at a 1 m distance away from the surface.
8) The surface was examined to ensure it was not over-saturated with coating solution.
9) The surface was allowed to dry for several hours.

In non-limiting embodiments, the surface substrate is dried at room temperature for 5 to 10 minutes and electrostatically sprayed with the second solution. In non-limiting embodiments, the sample can be cured overnight at room temperature or cured in an oven at 90° C. for 30 min to increase the stability of the coating through the interaction of titanium and the catechol moiety.

A second glass slide was treated according to the protocol described above, except the surface was coated with the biocidal polymer solution first, followed by the titanium anatase phase.

In both methods, it was observed that the coating resisted repeated manual friction (over 20 cycles) using both wetted and dry paper towel, cotton-based cloth, and polyester/polypropylene wipes.

### Visual Fluorescein Test

Treated and control glass slides were transferred to a 1% fluorescein solution for 10 minutes. Subsequently, the samples were thoroughly washed with distilled water until there was no additional release of physisorbed fluorescein from the samples. The samples were then air dried. Orange color was observed on the surfaces due to the retained fluorescein dye molecules bound to quaternary amino groups from both catecholacetyl-coalkyl PVP and octadecyl(4-catecholacetyl)dimethylammonium chloride.

The fluorescein test showed that treated surfaces retained their orange color before and after manual friction, which means that the coating resisted moderate abrasion. These results were observed on the slide where the biocidal polymer coating solution was sprayed after the titanium anatase solution, but not on the slide where the biocidal polymer coating solution was sprayed before the titanium anatase solution. While not wishing to be bound by theory, this result may be due to the underlying PVP with cationic charges being shielded from fluorescein due to the anatase layer.

In order to examine whether silane-based compounds would also be useful in these coatings, an ethanolic solution of 1% octadecyldimethyl(3-trimethoxysilylpropyl)ammonium chloride was prepared as a spray-on coating as well as a mixture of peroxotitanic acid and peroxo-modified anatase sol 0.8%. Each solution was electrostatically sprayed onto glass slides sequentially with the titanium anatase sprayed first followed by the solution comprising the octadecyldimethyl(3-trimethoxysilylpropyl)ammonium chloride, and the slides were cured overnight at room temperature according the protocol described above.

It was observed that the silane-based coating did not resist manual friction using wetted paper towel, cotton-based cloth, and polyester/polypropylene wipes. The entire coating was easily removed from the glass slide after only three cycles, indicating a lack of durability and robustness of the layer. Although not wishing to be bound by theory, this result suggests that the electrostatic interactions between catechol moieties and surfaces is superior to the electrostatic interactions displayed by organosilanes.

The fluorescein test showed that after extensive washing of the glass slides with water, the fluorescein was completely removed from the surfaces that underwent three cycles of manual friction, indicating that the coating comprising the silane-based compound did not resist minor abrasion and that the quaternary ammonium compound was removed from the surface.

### Example 32: Grafting on Filter Paper of Acetylcatechol-Co-Hexyl-PEI

### Deposition and Grafting

A 1 cm² dry filter paper sample was impregnated with 4 drops of aqueous 1% acetylcatechol-co-hexyl-PEI (synthesized according to Example 20 with a hexyl moiety instead of a decyl moiety) and grafted according to Example 4.

### Visual Fluorescein Test

Samples were treated according to the visual fluorescein test described in Example 4. The orange appearance of acetylcatechol-co-hexyl-PEI-treated filter paper is due to the high number of fluorescein dye molecules bound to the quaternary amino groups of the acetylcatechol-co-hexyl-PEI, which is covalently attached to the cotton.

### Example 33: Grafting on Glass of Acetylcatechol-Co-Hexyl-PEI

### Glass Preparation and Activation

Glass slides were prepared according to Example 6.

### Depositing and Grafting

The solution prepared according to Example 20 was deposited on the activated glass coverslips by spin-coating, and then covalently grafted on the activated glass.

### Surface Charge Determination by Fluorescein Test

Measurements were as follows:
Spin-coated treated glass slide (150 μL deposit, 4000 RPM, 40 seconds, 3 mL CTAB/PBS)
Calculated surface cationic density using 750 kda PEI as a reageant: 2.1±0.4×10¹⁶ cations/cm²
Calculated surface cationic density using 25 kda PEI as a reageant: 3.4±0.2×10¹⁴ cations/cm²

These results confirmed the effective grafting of the catechol-based quaternary ammonium moiety on glass.

### Example 33: Grafting on Filter Paper of 3-Trimethoxypropyl-Cohexyl-PEI

### Deposition and Grafting

A 1 cm² dry filter paper sample was impregnated with 4 drops of aqueous 1% with 3-trimethoxypropyl-cohexyl-PEI (synthesized according to Example 21 and grafted according to Example 4.

### Visual Fluorescein Test

Samples were treated according to the visual fluorescein test described in Example 4. The orange appearance of 3-trimethoxypropyl-cohexyl-PEI treated filter paper is due to the high number of fluorescein dye molecules bound to the quaternary amino groups of the 3-trimethoxypropyl-cohexyl-PEI, which is covalently grafted to the cotton.

### Example 34: Grafting on glass of 3-trimethoxypropyl-cohexyl-PEI

### Glass Preparation and Activation

Glass preparation was performed according to Example 6.

### Depositing and Grafting

The solution prepared according to Example 21 was deposited on the activated glass coverslips by spin-coating, and then covalently grafted on the activated glass according to Example 6.

### Surface Charge Determination by Fluorescein Test

Surface cationic density (N+/cm2) was calculated by a fluorescein test as described in Example 6.

Measurements were as follows:
Spin-coated treated glass slide (150 μL deposit, 4000 RPM, 40 seconds, 3 mL CTAB/PBS)
Calculated surface cationic density using 750 kda PEI as a reageant: 3.6±0.3×10¹⁶ cations/cm²
Calculated surface cationic density using 25 kda PEI as a reageant: 4.2±0.4×10¹⁴ cations/cm²

These results confirmed the effective grafting of the silane-based quaternized methyl PEI on glass.

### Example 35: Grafting on Filter Paper of Quaternized Bis(3-Trimethoxysilylpropyl)-N-Methyl-N-Paraxylyl-Cobutyl-PVP

### Deposition and Grafting

A 1 cm² dry filter paper sample was impregnated with 4 drops of aqueous 1% quaternized bis(3-trimethoxysilylpropyl)-N-methyl-N-paraxylyl-cobutyl-PVP (synthesized according to Example 29) and grafted according to Example 4.

### Visual Fluorescein Test

Samples were treated according to the visual fluorescein test described in Example 4. The orange appearance of quaternized bis(3-trimethoxysilylpropyl)-N-methyl-N-paraxylyl-cobutyl-PVP treated filter paper is due to the high number of fluorescein dye molecules bound to the quaternary amino groups of the quaternized bis(3-trimethoxysilylpropyl)-N-methyl-N-paraxylyl-cobutyl-PVP, which is covalently attached to the cotton.

### Example 36: Grafting on Glass of Quaternized Bis(3-Trimethoxysilylpropyl)-N-Methyl-N-Paraxylyl-Cobutyl-PVP

### Glass Preparation and Activation

Glass preparation was performed according to Example 6.

### Depositing and Grafting

The solution prepared according to Example 29 was deposited on the activated glass coverslips by spin-coating, and then covalently grafted on the activated glass according to Example 6.

### Surface Charge Determination by Fluorescein Test

Surface cationic density (N⁺/cm²) was calculated by a fluorescein test as described in Example 6.

Measurements were as follows:
Spin-coated treated glass slide (150 μL deposit, 4000 RPM, 40 seconds, 3 mL CTAB PBS)

$$Calculated surface cationic density : 9.3 \pm 0.5 \times {10}^{15} cations / {cm}^{2}$$

These results confirmed the effective grafting of the dipodal silane-based polycations on glass.

### Example 37: Grafting on Glass of Bis(3-Trimethoxysilyl)Propyl-N,N-Dihexadecyl Ammonium Bromide

### Glass Preparation and Activation

Glass preparation was performed according to Example 6.

### Depositing and Grafting

The solution prepared according to Example 28 (bis(3-trimethoxysilyl)propyl-N,N-dihexadecyl ammonium bromide) was deposited on the activated glass coverslips by spin-coating, and then covalently grafted on the activated glass according to Example 6.

### Surface Charge Determination by Fluorescein Test

Surface cationic density (N+/cm2) was calculated by a fluorescein test as described in Example 6.

Measurements were as follows:
Spin-coated treated glass slide (150 μL deposit, 4000 RPM, 40 seconds, 3 mL CTAB/PBS)

$$Calculated surface cationic density : 2.7 \pm 02 \times {10}^{14} cations / {cm}^{2}$$

These results confirmed the effective grafting of the dipodal silane-based quaternary ammonium alkyl chain on glass.

### Example 38: Grafting of L-Cysteine on Filter Paper Using 4-Iodoacetylcatechol as a Linker

### Deposition and Grafting

A 1 cm² dry filter paper sample was impregnated with 4 drops of acetonic 4-iodoacetylcatechol (Synthesized according to Example 1, Scheme 3 and after filtration of the NaCl product) and baked in an oven at 110° C. for 60 minutes. A control filter paper sample was impregnated with distilled water. Samples were then sonicated in ethanol for 10 minutes to remove any excess of reagent and air-dried. The filter paper sample was immersed in a 10× Phosphate Buffer Saline (PBS) containing 500 mg of L-cysteine. The solution was stirred overnight at room temperature and protected from light. The filter paper sample was removed from the solution and sonicated for 10 minutes in deionized water. It was then covered by a 1% aqueous ninhydrin solution and heated at 90° C. for 10 minutes.

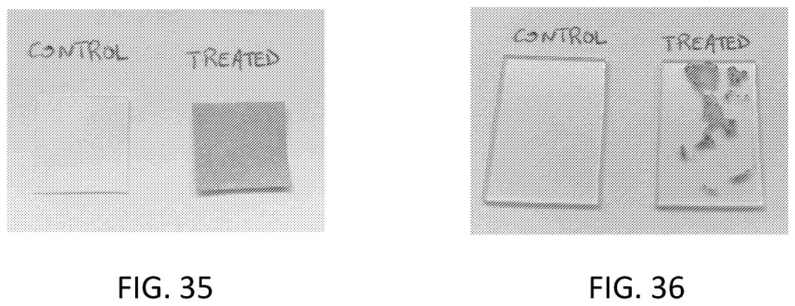

The treated filter paper appeared purple after ninhydrin dye was applied and the control paper remained white after ninhydrin dye application (FIG. 35). This result demonstrates the presence of grafted L-cysteine on the filter paper.

### Example 39: Grafting of L-Cysteine on Glass Using 4-Iodoacetylcatechol as a Linker

### Glass Preparation and Activation

Glass preparation was performed according to Example 6.

### Depositing and Grafting

The activated glass was immersed in the solution prepared according to Example 1, Scheme 3 and after filtration of the NaCl product. Immersed samples were transferred to an oven at 110° C. for 60 minutes. Samples were then retrieved and sonicated in ethanol for 5 minutes for adequate removal of any remaining physisorbed reagent. Samples were then air-dried. The glass slide was immersed in a 10× Phosphate Buffer Saline (PBS) containing 500 mg of L-cysteine. The solution was stirred overnight at room temperature. The glass slide was removed from the solution and sonicated for 10 minutes in deionized water. It was then covered by a 1% aqueous ninhydrin solution and heated at 90° C. for 10 minutes. Purple-blue spots appeared on the surface which proves the presence of grafted L-cysteine on the glass surface. As a comparative tool, a control glass slide was sonicated in ethanol for 10 minutes and air-dried. The glass slide was covered with 1% aqueous ninhydrin solution and heated at 90° C. for 10 minutes. No spots appeared on the surface of the control glass slide.

These results confirmed the effective grafting of an amino-acid such as L-cysteine using a iodocatechol linker.

### Example 40: Grafting Examples

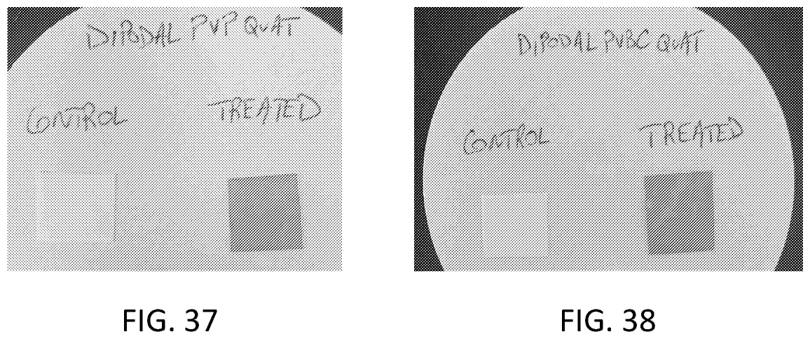

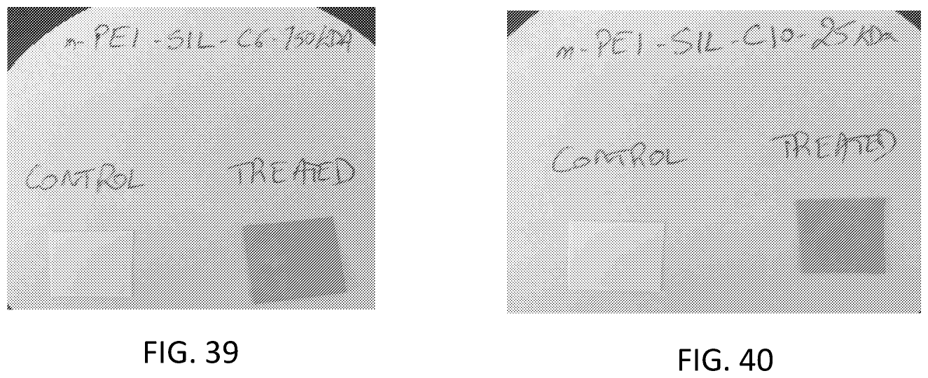

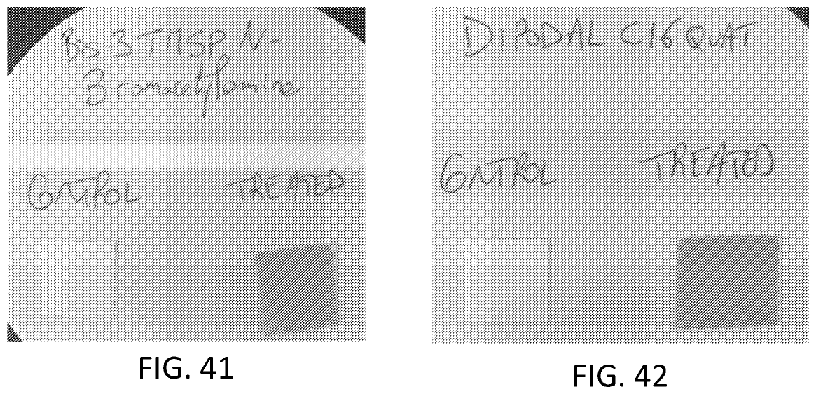

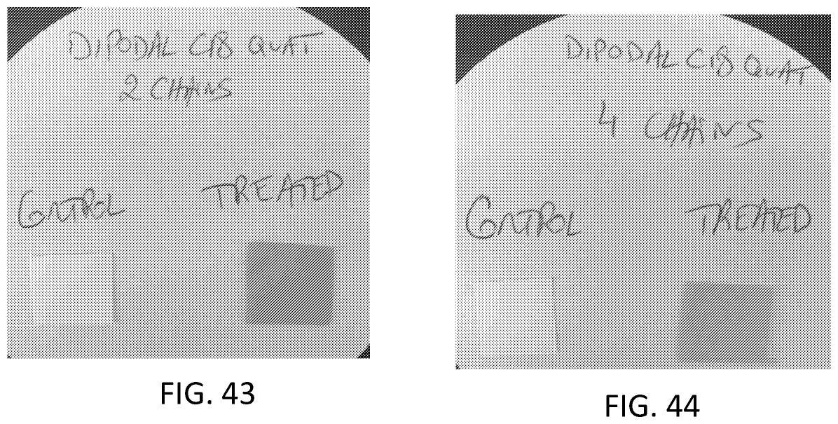

FIG. 37-FIG. 44 illustrate successful grafting of polymers and compounds of the disclosure onto filter paper. FIG. 37 illustrates a comparison between control filter paper and treated filter paper with quaternized bis(3-trimethoxysilylpropyl)-N-methyl-N-paraxylyl-cobutyl-PVP. FIG. 38 illustrates a comparison between control filter paper and treated filter paper with poly(vinylbenzyl chloride) partially quaternized with bis(N-methyl)₃-propyltrimethoxysilane groups and N,N-dimethylbutyl groups. FIG. 39 illustrates a comparison between control filter paper and treated filter paper with 3-trimethoxypropyl-cohexyl-PEI (from PEI at 750 kda). FIG. 40 illustrates a comparison between control filter paper and treated filter paper with 3-trimethoxypropyl-codecyl-PEI (from PEI at 25 kda). FIG. 41 illustrates a comparison between control filter paper and treated filter paper with bis(3-trimethoxysilypropyl)-N-bromoacetylamine. FIG. 42 illustrates a comparison between control filter paper and treated filter paper with bis(3-trimethoxysilyl)propyl-N,N-dihexadecyl ammonium bromide. FIG. 43 illustrates a comparison between control filter paper and treated filter paper with bis(3-trimethoxysilylpropyl)-N,N-methylalkylammonium bromide. FIG. 44 illustrates a comparison between control filter paper and treated filter paper with bis[3-(trimethoxysilyl)propyl-N,N′-tetraalkylethylenediammonium.

### REFERENCES

1. Kügler R., Bouloussa O., Rondelez F. Evidence of a Charge-Density Threshold for Optimum Efficiency of Biocidal Cationic Surfaces. Microbiology 2005 May; 151(Pt 5):1341-1348.
2. Oh J. Y., Cho I. H., Lee H., Park K-J., Lee H., Park S. Y. 2012, Bio-inspired catechol chemistry: a new way to develop a rer-moldable and injectable coacervate hydrogel. Chem. Comm. 48:11895-11897.
3. Elena P., Miri K. 2018, Formation of contact active antimicrobial surfaces by covalent grafting of quaternary ammonium compounds. Colloids and Surfaces B: Biointerfaces 169:195-205.
4. Kim S., Nam J. A., Lee S., In I., Park, S. Y. 2014, Antimicrobial activity of water resistant surface coating from catechol conjugated polyquaternary amine on versatile substrates. J. Appl. Polym. Sci. 131:40708.
5. Kim S. H., Lee S., In I., Park, S. Y. 2016, Synthesis and antibacterial activity of surface coated catechol-conjugated polymer with silver nanoparticles on versatile substrate. Surf. Interface Anal. 9:995-1001.
6. Le T.-N., Au-Duong A.-N., Lee C.-K. 2019, Facile coating on microporous polypropylene membrane for antifouling microfiltration using comb-shaped poly(N-vinaylpyrrolidone) with multivalent catechol. J. Membrane Science 574:164-173.
7. Wu Z., Wang J., Pei D., Li L., Mu Y., Wan X. 2018, A simple strategy to achieve mussel-inspired highly effective antibacterial coating. Macromol. Mater. Eng. 303:1700430.
8. Nam J. A., Nahain A.-A., Kim S. M., In I., Park S. Y. 2013, Successful stabilization of functionalized hybrid graphene for high-performance antimicrobial activity. Acta Biomaterialia 9:7996-8003.
9. Jeong C. J., In, I. Park S. Y. 2014, Facile preparation of metal nanoparticle-coated polystyrene beads by catechol conjugated polymer. Surf Interface Anal. 47:253-258.
10. Arkles, B., Pan, Y., Larson, G. and Singh, M., 2020. Enhanced Hydrolytic Stability Of Siliceous Surfaces Modified With Pendant Dipodal Silanes. M Chem Eur J. 2014, 20, 9442-9450.
11. Ichinose et al., “Properties of Anastase Films for Photocatalyst from Peroxotitanic Acid Solution and Peroxo-Modified Anatase Sol,” Journal of the Cermanic Society of Japan, International Edition, vol. 104, No. 10 (October 1996) pp. 909-912.
12. Ichinose et al., “Synthesis of Peroxo-Modified Anatase Sol Peroxo Titanic Acid Solution,” Journal of the Ceramic Society of Japan, International Edition, vol. 104, No. 8 (Auyg. 1996) pp. 697-700.

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

1. A polymer comprising at least one moiety of formula (XVIIa): 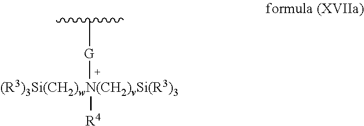
wherein in formula (XVIIa):
G is a single bond or a linking group;
each R³ is independently optionally substituted alkoxy;
R⁴ is independently optionally substituted alkyl;
v is an integer from 3 to 10; and
w is an integer from 3 to 10.

2. The polymer of claim 1, wherein the polymer comprises at least one moiety of formula (XVIIb): 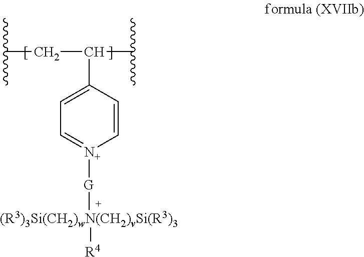
wherein in formula (XVIIb):
G is a single bond or a linking group;
each R³ is independently optionally substituted alkoxy;
R⁴ is independently optionally substituted alkyl;
v is an integer from 3 to 10; and
w is an integer from 3 to 10.

3. (canceled)

4. The polymer of claim 1, wherein the polymer further comprises at least one moiety of formula (III): 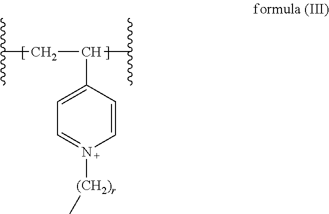
wherein in formula (III):
r is an integer from 3 to 20.

5. The polymer of claim 1, wherein the polymer comprises at least one moiety of formula (XVIIc): 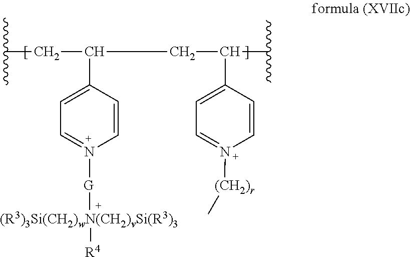
wherein in formula (XVIIc):
G is a single bond or a linking group;
each R³ is independently optionally substituted alkoxy;
R⁴ is independently optionally substituted alkyl;
r is an integer from 3 to 11;
v is an integer from 3 to 10; and
w is an integer from 3 to 10.

6-8. (canceled)

9. The polymer of claim 1, wherein the polymer comprises at least one moiety of formula (XVIIh):

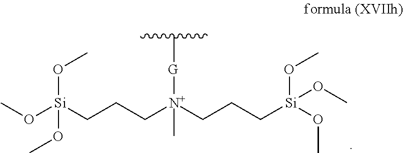

10.-11. (canceled)

12. A polymer comprising at least one moiety of formula (XXI): 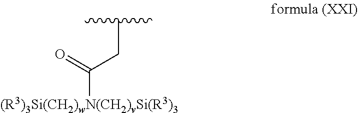
wherein in formula (XXI):
each R³ is independently optionally substituted alkoxy;
v is an integer from 3 to 10; and
w is an integer from 3 to 10.

13. The polymer of claim 12, wherein the polymer comprises at least one moiety of formula (XXIa): 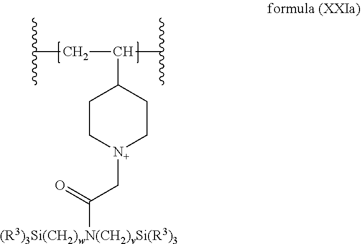
wherein in formula (XXIa):
each R³ is independently optionally substituted alkoxy;
v is an integer from 3 to 10; and
w is an integer from 3 to 10.

14. The polymer of claim 12, wherein the polymer comprises at least one moiety of formula (XXIb):

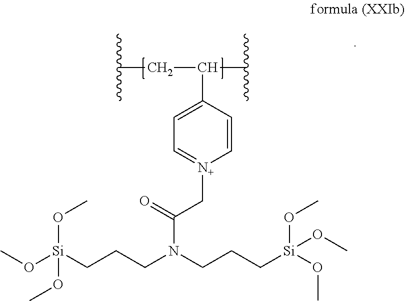

15. The polymer of claim 12, wherein the polymer further comprises at least one moiety of formula (III): 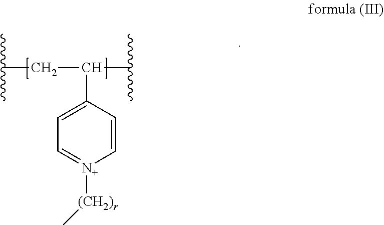
wherein r is an integer from 3 to 20.

16. The polymer of claim 12, wherein the polymer comprises at least one moiety of formula (XXId): 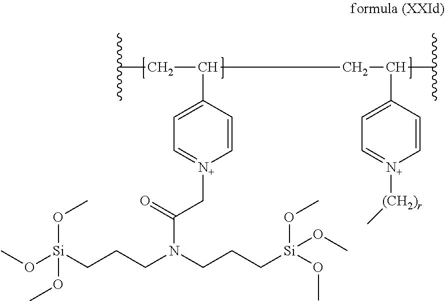
wherein in formula (XXId):
r is an integer from 3 to 11.

17.-27. (canceled)

28. The polymer of claim 1, wherein the polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide), poly(diallyldimethylammonium), or a C₃-C₂₂ alkyne.

29.-50. (canceled)

51. A polyethylenimine (PEI) polymer comprising at least one moiety of formula (XIa): 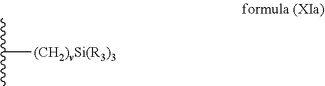
wherein in formula (XIa):
each R³ is independently optionally substituted alkoxy; and
v is an integer from 3 to 10.

52. The PEI polymer of claim 51, wherein the PEI polymer comprises at least one of the following moieties of formula (XIa): 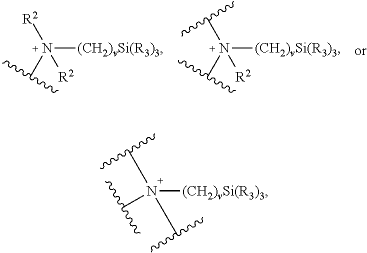
wherein each R² is independently optionally substituted alkyl, optionally wherein R² is methyl.

53. The PEI polymer of claim 51, wherein the moiety of formula (XIa) is a moiety of formula (XIb), or any substructure thereof: 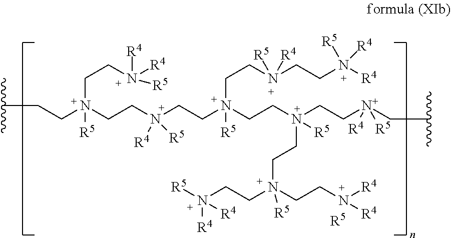
wherein in formula (XIb):
each R⁴ is independently optionally substituted alkyl; and
each R⁵ is independently optionally substituted alkyl or a moiety of formula (XIa): 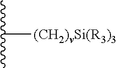
each R³ is independently optionally substituted alkoxy; and
v is an integer from 3 to 10,
with the proviso that at least one R⁵ is a moiety of formula (XIa):

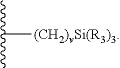

54. The PEI polymer of claim 53, wherein the moiety of formula (XIa) is a moiety of formula (XIc), or any substructure thereof: 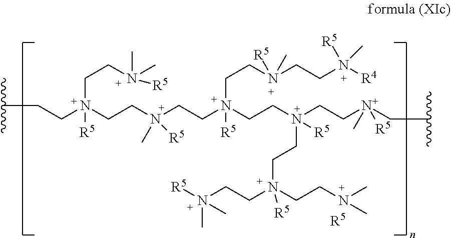
wherein in formula (XIc):
each R⁵ is independently C₆ alkyl or 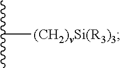
each R³ is independently optionally substituted alkoxy; and
v is an integer from 3 to 10,
with the proviso that at least one R⁵ is

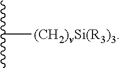

55. The PEI polymer of claim 51, wherein each moiety of formula (XIa) 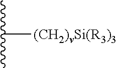
is

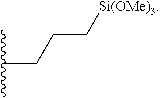

56. The PEI polymer of claim 51, wherein the molar ratio of formula (XIa) is 0.05≤x≤0.2.

57. A surface having the PEI polymer of claim 51 grafted thereon.

58. A method of controlling the growth of at least one bacteria, fungi, protozoa, or virus, the method comprising grafting a polymer of claim 51 onto a surface.

59. The polymer of claim 12, wherein the polymer comprises polyvinylpyridine (PVP), polyvinylbenzylchloride, polyethylenimine (PEI), propynyl methacrylate, polyethylene, polyacrylamide, polystyrene, polyvinylalcohol, polyallylamine, polyallylalcohol, polyvinylbenzyl, polyamine, polymethacrylate, polyether, poly(ethylene-alt-succinimide), poly(diallyldimethylammonium), or a C₃-C₂₂ alkyne.
